(19) United States
(12) Patent Application Publication
(10) Pub. No.: US 2023/0157998 A1
(43) Pub. Date: May 25, 2023

(54) Methods of Inhibiting SARS-CoV-2 Replication and Treating Corona Virus Disease 2019

(71) Applicant: Pfizer Inc., New York, NY (US)

(72) Inventors: Padmavani Bezawada, Veron Hills, IL (US); Benjamin Joseph Burke, San Diego, CA (US); Emma Louise Hawking, Sandwich, Kent (GB); Robert Louis Hoffman, San Diego, CA (US); Robert Steven Kania, Del Mar, CA (US); Jonathan Richard Lillis, Sandwich, Kent (GB); Matthew Nathan O'Brien Laramy, Evanston, IL (US); Klimentina Dimitrova Pencheva, Sandwich, Kent (GB); Bradley Paul Sullivan, Gurnee, IL (US); Andrew John Thiel, Gurnee, IL (US); Martyn David Ticehurst, Sandwich, Kent (GB)

(73) Assignee: Pfizer Inc., New York, NY (US)

(21) Appl. No.: 17/905,686

(22) PCT Filed: Mar. 3, 2021
(86) PCT No.: PCT/IB2021/051768

§ 371 Date: Sep. 6, 2022

Related U.S. Application Data
(60) Provisional application No. 63/149,434, filed on Feb. 15, 2021.
(60) Provisional application No. 63/065,644, filed on Aug. 14, 2020.
(60) Provisional application No. 63/038,502, filed on Jun. 12, 2020.
(60) Provisional application No. 62/986,538, filed on Mar. 6, 2020.

(51) Int. Cl. A61K 31/405; A61K 31/706; A61K 31/7052; A61K 9/00

(52) U.S. Cl. CPC A61K 31/405; A61K 31/706; A61K 31/7052; A61K 9/0019

(57) ABSTRACT

The invention relates to methods of treating COVID-19 in a patient by administering therapeutically effective amounts of certain SARS-CoV-2 inhibitor compounds or pharmaceutical compositions containing them to a patient in need thereof. The invention also relates to inhibiting SARS-CoV-2 coronavirus viral replication activity comprising contacting SARS-CoV-2-related coronavirus protease with a therapeutically effective amount of a SARS-Cov-2 protease inhibitor, such as a SARS-Cov-2 3CL protease inhibitor and compositions comprising the same

### BACKGROUND OF THE INVENTION

The invention relates to methods of inhibiting viral replication activity comprising contacting a SARS-CoV-2-related 3C-like (“3CL”) protease with a therapeutically effective amount of a SARS-CoV-2-related 3C-like protease inhibitor. The invention also relates to methods of treating Coronavirus Disease 2019 (“COVID-19”) in a patient by administering a therapeutically effective amount of a SARS-CoV-2-related 3C-like protease inhibitor to a patient in need thereof. The invention further relates to methods of treating COVID-19 in a patient, the method comprising administering a pharmaceutical composition comprising a therapeutically effective amount of the SARS-CoV-2-related 3C-like protease inhibitor to a patient in need thereof.

A worldwide outbreak of Coronavirus Disease 2019 (“COVID-19”) has been associated with exposures originating in December 2019 in Wuhan, Hubei Province, China. By early March 2020 the outbreak of COVID-19 has spread to numerous countries worldwide including the United States with over 93,000 people having been confirmed as infected and resulting in over 3,000 deaths. The causative agent for COVID-19 has been identified as a novel coronavirus which has been named Severe Acute Respiratory Syndrome Corona Virus 2 (“SARS-CoV-2”). The genome sequence of SARS-CoV-2 has been sequenced from isolates obtained from nine patients in Wuhan, China and has been found to be of the subgenus Sarbecovirus of the genus Betacoronovirus. Lu, R. et al. The Lancet, Jan. 29, 2020; http://doi.org/10.1016/S0140-6736(20.). The sequence of SARS-CoV-2 was found to have 88% homology with two bat-derived SARS-like coronaviruses, bat-SL-CoVZC45 and bat-SL-CoVZXC21 which were collected in 2018 in Zhoushan, eastern China. SARS-CoV-2 was also found to share about 79% homology with Severe Acute Respiratory Syndrome Corona Virus (“SARS-CoV”), the causative agent of the SARS outbreak in 2002-2003, and about 50% homology with Middle East Respiratory Syndrome Coronavirus (“MERS-CoV”), the causative agent of a respiratory viral outbreak originating in the Middle East in 2012. Based on a recent analysis of 103 sequenced genomes of SARS-CoV-2 it has been proposed that SARS-CoV-2 can be divided into two major types (L and S types) with the S type being ancestral and the L type having evolved from the S-type. Lu, J.; Cui, J. et al. On the origin and continuing evolution of SARS-CoV-2; http://doi.org/10.1093/nsr/nwaa036. The S and L types can be clearly defined by just two tightly linked SNPs at positions 8,782 (orf1ab:T8517C, synonymous) and 28,144 (ORF8: C251T, S84L). In the 103 genomes analyzed approximately 70% were of the L-type and approximately 30% were of the S-type. It is unclear if the evolution of the L-type from the S-type occurred in humans or through a zoonotic intermediate but it appears that the L-type is more aggressive than the S-type and human interference in attempting to contain the outbreak may have shifted the relative abundance of the L and S types soon after the SARS-CoV-2 outbreak began. The discovery of the proposed S- and L-subtypes of SARS-CoV-2 raises the possibility that an individual could potentially be infected sequentially with the individual subtypes or be infected with both subtypes at the same time. In view of this evolving threat there is an acute need in the art for an effective treatment for COVID-19 and for methods of inhibiting replication of the SARS-CoV-2 coronavirus.

Recent evidence clearly shows that the newly emerged coronavirus SARS-CoV-2, the causative agent of COVID-19 (Centers for Disease Control, CDC) has acquired the ability of human to human transmission leading to community spread of the virus. The sequence of the SARS-CoV-2 receptor binding domain (“RBD”), including its receptor-binding motif (RBM) that directly contacts the angiotensin 2 receptor, ACE2, is similar to the RBD and RBM of SARS-CoV, strongly suggesting that SARS-CoV-2 uses ACE2 as its receptor. Yushun Wan, Y.; Shang, J.; Graham, R.; 2, Baric, R. S.; Li, F.; Receptor recognition by novel coronavirus from Wuhan: An analysis based on decade-long structural studies of SARS; J. Virol. 2020; doi:10.1128/JVI.00127-20. Several critical residues in SARS-CoV-2 RBM (particularly Gln⁴⁹³) provide favorable interactions with human ACE2, consistent with SARS-CoV-2's capacity for human cell infection. Several other critical residues in SARS-CoV-2's RBM (particularly Asn⁵⁰¹) are compatible with, but not ideal for, binding human ACE2, suggesting that SARS-CoV-2 uses ACE2 binding in some capacity for human-to-human transmission.

Coronavirus replication and transcription function is encoded by the so-called “replicase” gene (Ziebuhr, J., Snijder, E. J., and Gorbaleya, A. E.; Virus-encoded proteinases and proteolytic processing in Nidovirales. J. Gen. Virol. 2000, 81, 853-879; and Fehr, A. R.; Perlman, S.; Coronaviruses: An Overview of Their Replication and Pathogenesis Methods Mol Biol. 2015; 1282: 1-23. doi:10.1007/978-1-4939-2438-7_1), which consists of two overlapping polyproteins that are extensively processed by viral proteases. The C-proximal region is processed at eleven conserved interdomain junctions by the coronavirus main or “3C-like” protease (Ziebuhr, Snijder, Gorbaleya, 2000 and Fehr, Perlman et al., 2015). The name “3C-like” protease derives from certain similarities between the coronavirus enzyme and the well-known picornavirus 3C proteases. These include substrate preferences, use of cysteine as an active site nucleophile in catalysis, and similarities in their putative overall polypeptide folds. The SARS-CoV-2 3CL protease sequence (Accession No. YP_009725301.1) has been found to share 96.08% homology when compared with the SARS-CoV 3CL protease (Accession No. YP_009725301.1) Xu, J.; Zhao, S.; Teng, T.; Abdalla, A. E.; Zhu, W.; Xie, L.; Wang, Y.; Guo, X.; Systematic Comparison of Two Animal-to-Human Transmitted Human Coronaviruses: SARS-CoV-2 and SARS-CoV; Viruses 2020, 12, 244; doi:10.3390/v12020244. Very recently Hilgenfeld and colleagues published a high-resolution X-ray structure of the SARS-CoV-2 coronavirus main protease (3CL) Zhang, L.; Lin, D.; Sun, X.; Rox, K.; Hilgenfeld, R.; X-ray Structure of Main Protease of the Novel Coronavirus SARS-CoV-2 Enables Design of a-Ketoamide Inhibitors; bioRxiv preprint doi: https//doi.org/10.1101/2020.02.17.952879. The structure indicates that there are differences when comparing the 3CL proteases of SARS-CoV-2 and SARS-CoV. In the SARS-CoV but not in the SARS-CoV-2 3CL protease dimer, there is a polar interaction between the two domains Ill involving a 2.60-Å hydrogen bond between the side-chain hydroxyl groups of residue Thr²⁸⁵ of each protomer, and supported by a hydrophobic contact between the side-chain of Ile²⁸⁶ and Thr²⁸⁵ Cγ₂. In the SARS-CoV-2 3CL, the threonine is replaced by alanine, and the isoleucine by leucine when compared with the same residues in the SARS-CoV 3CL. The Thr285Ala replacement observed in the SARS-CoV-2 3CL protease allows the two domains Ill to approach each other somewhat closer (the distance between the Ca atoms of residues 285 in molecules A and B is 6.77 Å in SARS-CoV 3CL protease and 5.21 Å in SARS-CoV-2 3CL protease and the distance between the centers of mass of the two domains III shrinks from 33.4 Å to 32.1 Å). In the active site of SARS-CoV-2 3CL Cys¹⁴⁵ and His⁴¹ form a catalytic dyad which when taken together with a with a buried water molecule that is hydrogen bonded to His⁴¹ can be considered to constitute a catalytic triad of the SARS-CoV-2 3CL protease. In view of the ongoing SARS-CoV-2 spread which has caused the current worldwide COVID-19 outbreak it is desirable to have new methods of inhibiting SARS-CoV-2 viral replication and of treating COVID-19 in patients.

### SUMMARY OF THE INVENTION

Embodiments (En) describe the invention, wherein E1 is a method of treating COVID-19 in a patient, the method comprising administering to a patient in need thereof a therapeutically effective amount of a compound selected from the group consisting of: (3S)-3-({4-methyl-N-[(2R)-tetrahydrofuran-2-ylcarbonyl]-L-leucyl}amino)-2-oxo-4-[(3S)-2-oxopyrrolidin-3-yl]butyl 2,6-dichlorobenzoate; (3S)-3-({N-[(4-methoxy-1H-indol-2-yl)carbonyl]-L-leucyl}amino)-2-oxo-4-[(3S)-2-oxopyrrolidin-3-yl]butyl cyclopropanecarboxylate; N—((S)-1-(((S)-1-(benzo[d]thiazol-2-yl)-1-oxo-3-((S)-2-oxopyrrolidin-3-yl)propan-2-yl)amino)-3-cyclopropyl-1-oxopropan-2-yl)picolinamide; N—((S)-1-(((S)-1-(benzo[d]thiazol-2-yl)-1-oxo-3-((S)-2-oxopyrrolidin-3-yl)propan-2-yl)amino)-3-cyclopentyl-1-oxopropan-2-yl)-4-methoxy-1H-indole-2-carboxamide; N—((S)-2-(((S)-1-(benzo[d]thiazol-2-yl)-1-oxo-3-((S)-2-oxopyrrolidin-3-yl)propan-2-yl)amino)-1-cyclopentyl-2-oxoethyl)-4-methoxy-1H-indole-2-carboxamide; N-((1S)-1-{[((1S)-3-hydroxy-2-oxo-1-{[(3S)-2-oxopyrrolidin-3-yl] methyl}propyl)amino]carbonyl}-3,3-dimethylbutyl)-1H-indole-2-carboxamide; N-((1S)-1-{[((1S)-3-hydroxy-2-oxo-1-{[(3S)-2-oxopyrrolidin-3-yl]methyl} propyl)amino] carbonyl}pentyl)-4-methoxy-1H-indole-2-carboxamide; N—((S)-1-(((S)-4-hydroxy-3-oxo-1-((S)-2-oxopyrrolidin-3-yl)butan-2-yl)amino)-1-oxo-3-phenylpropan-2-yl)-4-methoxy-1H-indole-2-carboxamide; N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide; (2R)-2-cyclopentyl-2-[2-(2,6-diethylpyridin-4-yl)ethyl]-5-[(5,7-dimethyl-[1,2,4] triazolo[1,5-a]pyrimidin-2-yl)methyl]-4-hydroxy-3H-pyran-6-one; (3S,4aS,8aS)—N-tert-butyl-2-[(2R,3R)-2-hydroxy-3-[(3-hydroxy-2-methylbenzoyl) amino]-4-phenylsulfanylbutyl]-3,4,4a,5,6,7,8,8a-octahydro-1H-isoquinoline-3-carboxamide; Ethyl (E,4S)-4-[[(2R,5S)-2-[(4-fluorophenyl)methyl]-6-methyl-5-[(5-methyl-1,2-oxazole-3-carbonyl)amino]-4-oxoheptanoyl]amino]-5-[(3S)-2-oxopyrrolidin-3-yl]pent-2-enoate; and (R)-3-((2S,3S)-2-Hydroxy-3-{[1-(3-hydroxy-2-methyl-phenyl)-methanoyl]-amino}-4-phenyl-butanoyl)-5,5-dimethyl-thiazolidine-4-carboxylic acid allylamide; or a pharmaceutically acceptable salt thereof.

E2 is the method of E1 wherein the compound is administered orally or intravenously. E3 is the method of E2 wherein the compound is administered intravenously. E4 is the method of E3 wherein the compound is administered intermittently over a 24-hour period or continuously over a 24-hour period. E5 is the method of any one of E1 through E4 wherein the compound is (3S)-3-({4-methyl-N-[(2R)-tetrahydrofuran-2-ylcarbonyl]-L-leucyl}amino)-2-oxo-4-[(3S)-2-oxopyrrolidin-3-yl]butyl 2,6-dichlorobenzoate; or a pharmaceutically acceptable salt thereof. E6 is the method of any one of E1 through E4 wherein the compound is (3S)-3-({N-[(4-methoxy-1H-indol-2-yl)carbonyl]-L-leucyl}amino)-2-oxo-4-[(3S)-2-oxopyrrolidin-3-yl]butyl cyclopropanecarboxylate; or a pharmaceutically acceptable salt thereof. E7 is the method of any one of E1 through E4 wherein the compound is N—((S)-1-(((S)-1-(benzo[d]thiazol-2-yl)-1-oxo-3-((S)-2-oxopyrrolidin-3-yl)propan-2-yl)amino)-3-cyclopropyl-1-oxopropan-2-yl)picolinamide; or a pharmaceutically acceptable salt thereof. E8 is the method of any one of E1 through E4 wherein the compound is N—((S)-1-(((S)-1-(benzo[d]thiazol-2-yl)-1-oxo-3-((S)-2-oxopyrrolidin-3-yl)propan-2-yl)amino)-3-cyclopentyl-1-oxopropan-2-yl)-4-methoxy-1H-indole-2-carboxamide; or a pharmaceutically acceptable salt thereof. E9 is the method of any one of any one of E1 through E4 wherein the compound is N—((S)-2-(((S)-1-(benzo[d]thiazol-2-yl)-1-oxo-3-((S)-2-oxopyrrolidin-3-yl)propan-2-yl)amino)-1-cyclopentyl-2-oxoethyl)-4-methoxy-1H-indole-2-carboxamide; or a pharmaceutically acceptable salt thereof. E10 is the method of any one of E1 through E4 wherein the compound is N-((1S)-1-{[((1S)-3-hydroxy-2-oxo-1-{[(3S)-2-oxopyrrolidin-3-yl]methyl}propyl)amino] carbonyl}-3,3-dimethylbutyl)-1H-indole-2-carboxamide; or a pharmaceutically acceptable salt thereof. E11 is the method of any one of any one of E1 through E4 wherein the compound is N-((1S)-1-{[((1S)-3-hydroxy-2-oxo-1-{[(3S)-2-oxopyrrolidin-3-yl]methyl} propyl)amino]carbonyl}pentyl)-4-methoxy-1H-indole-2-carboxamide; or a pharmaceutically acceptable salt thereof. E12 is the method of any one of any one of E1 through E4 wherein the compound is N—((S)-1-(((S)-4-hydroxy-3-oxo-1-((S)-2-oxopyrrolidin-3-yl)butan-2-yl)amino)-1-oxo-3-phenylpropan-2-yl)-4-methoxy-1H-indole-2-carboxamide; or a pharmaceutically acceptable salt thereof. E13 is the method of any one of any one of E1 through E4 wherein the compound is N-((1S)-1-{[((1S)-3-hydroxy-2-oxo-1-{[(3S)-2-oxopyrrolidin-3-yl]methyl}propyl)amino] carbonyl}-3-methylbutyl)-4-methoxy-1H-indole-2-carboxamide; or a pharmaceutically acceptable salt thereof. E14 is the method of any one of any one of E1 through E4 wherein the compound is (2R)-2-cyclopentyl-2-[2-(2,6-diethylpyridin-4-yl)ethyl]-5-[(5,7-dimethyl-[1,2,4] triazolo[1,5-a]pyrimidin-2-yl)methyl]-4-hydroxy-3H-pyran-6-one; or a pharmaceutically acceptable salt thereof. E15 is the method of any one of any one of E1 through E4 wherein the compound is (3S,4aS,8aS)—N-tert-butyl-2-[(2R,3R)-2-hydroxy-3-[(3-hydroxy-2-methylbenzoyl) amino]-4-phenylsulfanylbutyl]-3,4,4a,5,6,7,8,8a-octahydro-1H-isoquinoline-3-carboxamide; or a pharmaceutically acceptable salt thereof. E16 is the method of any one of any one of E1 through E4 wherein the compound is Ethyl (E,4S)-4-[[(2R,5S)-2-[(4-fluorophenyl)methyl]-6-methyl-5-[(5-methyl-1,2-oxazole-3-carbonyl)amino]-4-oxoheptanoyl]amino]-5-[(3S)-2-oxopyrrolidin-3-yl]pent-2-enoate; or a pharmaceutically acceptable salt thereof. E17 is the method of any one of E1 through E4 wherein the compound is (R)-3-((2S,3S)-2-Hydroxy-3-{[1-(3-hydroxy-2-methyl-phenyl)-methanoyl]-amino}-4-phenyl-butanoyl)-5,5-dimethyl-thiazolidine-4-carboxylic acid allylamide; or a pharmaceutically acceptable salt thereof.

E18 is a method of inhibiting or preventing SARS-CoV-2 viral replication comprising contacting a SARS-CoV-2 coronavirus protease with a therapeutically effective amount of a compound selected from the group consisting of: (3S)-3-({4-methyl-N-[(2R)-tetrahydrofuran-2-ylcarbonyl]-L-leucyl}amino)-2-oxo-4-[(3S)-2-oxopyrrolidin-3-yl]butyl 2,6-dichlorobenzoate; (3S)-3-({N-[(4-methoxy-1H-indol-2-yl)carbonyl]-L-leucyl}amino)-2-oxo-4-[(3S)-2-oxopyrrolidin-3-yl]butyl cyclopropanecarboxylate; N—((S)-1-(((S)-1-(benzo[d]thiazol-2-yl)-1-oxo-3-((S)-2-oxopyrrolidin-3-yl)propan-2-yl)amino)-3-cyclopropyl-1-oxopropan-2-yl)picolinamide; N—((S)-1-(((S)-1-(benzo[d]thiazol-2-yl)-1-oxo-3-((S)-2-oxopyrrolidin-3-yl)propan-2-yl)amino)-3-cyclopentyl-1-oxopropan-2-yl)-4-methoxy-1H-indole-2-carboxamide; N—((S)-2-(((S)-1-(benzo[d]thiazol-2-yl)-1-oxo-3-((S)-2-oxopyrrolidin-3-yl)propan-2-yl)amino)-1-cyclopentyl-2-oxoethyl)-4-methoxy-1H-indole-2-carboxamide; N-((1S)-1-{[((1S)-3-hydroxy-2-oxo-1-{[(3S)-2-oxopyrrolidin-3-yl]methyl}propyl)amino]carbonyl}-3,3-dimethylbutyl)-1H-indole-2-carboxamide; N-((1S)-1-{[((1S)-3-hydroxy-2-oxo-1-{[(3S)-2-oxopyrrolidin-3-yl]methyl} propyl)amino]carbonyl}pentyl)-4-methoxy-1H-indole-2-carboxamide; N—((S)-1-(((S)-4-hydroxy-3-oxo-1-((S)-2-oxopyrrolidin-3-yl)butan-2-yl)amino)-1-oxo-3-phenylpropan-2-yl)-4-methoxy-1H-indole-2-carboxamide; N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide; (2R)-2-cyclopentyl-2-[2-(2,6-diethylpyridin-4-yl)ethyl]-5-[(5,7-dimethyl-[1,2,4]triazolo[1,5-a]pyrimidin-2-yl)methyl]-4-hydroxy-3H-pyran-6-one; (3S,4aS,8aS)—N-tert-butyl-2-[(2R,3R)-2-hydroxy-3-[(3-hydroxy-2-methylbenzoyl) amino]-4-phenylsulfanylbutyl]-3,4,4a,5,6,7,8,8a-octahydro-1H-isoquinoline-3-carboxamide; Ethyl (E,4S)-4-[[(2R,5S)-2-[(4-fluorophenyl)methyl]-6-methyl-5-[(5-methyl-1,2-oxazole-3-carbonyl)amino]-4-oxoheptanoyl]amino]-5-[(3S)-2-oxopyrrolidin-3-yl]pent-2-enoate; and (R)-3-((2S,3S)-2-Hydroxy-3-{[1-(3-hydroxy-2-methyl-phenyl)-methanoyl]-amino}-4-phenyl-butanoyl)-5,5-dimethyl-thiazolidine-4-carboxylic acid allylamide; or a pharmaceutically acceptable salt thereof.

E19 is a method of inhibiting or preventing SARS-CoV-2 viral replication comprising contacting the SARS-CoV-2 coronavirus 3CL protease with a therapeutically effective amount of a compound selected from the group consisting of: (3S)-3-({4-methyl-N-[(2R)-tetrahydrofuran-2-ylcarbonyl]-L-leucyl}amino)-2-oxo-4-[(3S)-2-oxopyrrolidin-3-yl]butyl 2,6-dichlorobenzoate; (3S)-3-({N-[(4-methoxy-1H-indol-2-yl)carbonyl]-L-leucyl}amino)-2-oxo-4-[(3S)-2-oxopyrrolidin-3-yl]butyl cyclopropanecarboxylate; N—((S)-1-(((S)-1-(benzo[d]thiazol-2-yl)-1-oxo-3-((S)-2-oxopyrrolidin-3-yl)propan-2-yl)amino)-3-cyclopropyl-1-oxopropan-2-yl)picolinamide; N—((S)-1-(((S)-1-(benzo[d]thiazol-2-yl)-1-oxo-3-((S)-2-oxopyrrolidin-3-yl)propan-2-yl)amino)-3-cyclopentyl-1-oxopropan-2-yl)-4-methoxy-1H-indole-2-carboxamide; N—((S)-2-(((S)-1-(benzo[d]thiazol-2-yl)-1-oxo-3-((S)-2-oxopyrrolidin-3-yl)propan-2-yl)amino)-1-cyclopentyl-2-oxoethyl)-4-methoxy-1H-indole-2-carboxamide; N-((1S)-1-{[((1S)-3-hydroxy-2-oxo-1-{[(3S)-2-oxopyrrolidin-3-yl]methyl}propyl)amino]carbonyl}-3,3-dimethylbutyl)-1H-indole-2-carboxamide; N-((1S)-1-{[((1S)-3-hydroxy-2-oxo-1-{[(3S)-2-oxopyrrolidin-3-yl]methyl} propyl)amino]carbonyl}pentyl)-4-methoxy-1H-indole-2-carboxamide; N—((S)-1-(((S)-4-hydroxy-3-oxo-1-((S)-2-oxopyrrolidin-3-yl)butan-2-yl)amino)-1-oxo-3-phenylpropan-2-yl)-4-methoxy-1H-indole-2-carboxamide; and N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide; or a pharmaceutically acceptable salt thereof.

E20 is a method of treating COVID-19 in a patient, the method comprising administering to a patient in need thereof a pharmaceutical composition, the pharmaceutical composition comprising a therapeutically effective amount of a compound selected from the group consisting of: (3S)-3-({4-methyl-N-[(2R)-tetrahydrofuran-2-ylcarbonyl]-L-leucyl}amino)-2-oxo-4-[(3S)-2-oxopyrrolidin-3-yl]butyl 2,6-dichlorobenzoate; (3S)-3-({N-[(4-methoxy-1H-indol-2-yl)carbonyl]-L-leucyl}amino)-2-oxo-4-[(3S)-2-oxopyrrolidin-3-yl]butyl cyclopropane carboxylate; N—((S)-1-(((S)-1-(benzo[d]thiazol-2-yl)-1-oxo-3-((S)-2-oxopyrrolidin-3-yl)propan-2-yl)amino)-3-cyclopropyl-1-oxopropan-2-yl)picolinamide; N—((S)-1-(((S)-1-(benzo[d]thiazol-2-yl)-1-oxo-3-((S)-2-oxopyrrolidin-3-yl)propan-2-yl)amino)-3-cyclopentyl-1-oxopropan-2-yl)-4-methoxy-1H-indole-2-carboxamide; N—((S)-2-(((S)-1-(benzo[d]thiazol-2-yl)-1-oxo-3-((S)-2-oxopyrrolidin-3-yl)propan-2-yl)amino)-1-cyclopentyl-2-oxoethyl)-4-methoxy-1H-indole-2-carboxamide; N-((1S)-1-{[((1S)-3-hydroxy-2-oxo-1-{[(3S)-2-oxopyrrolidin-3-yl]methyl}propyl) amino]carbonyl}-3,3-dimethylbutyl)-1H-indole-2-carboxamide; N-((1S)-1-{[((1S)-3-hydroxy-2-oxo-1-{[(3S)-2-oxopyrrolidin-3-yl]methyl} propyl)amino]carbonyl}pentyl)-4-methoxy-1H-indole-2-carboxamide; N—((S)-1-(((S)-4-hydroxy-3-oxo-1-((S)-2-oxopyrrolidin-3-yl)butan-2-yl)amino)-1-oxo-3-phenylpropan-2-yl)-4-methoxy-1H-indole-2-carboxamide; N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide; (2R)-2-cyclopentyl-2-[2-(2,6-diethylpyridin-4-yl)ethyl]-5-[(5,7-dimethyl-[1,2,4]triazolo[1,5-a]pyrimidin-2-yl)methyl]-4-hydroxy-3H-pyran-6-one; (3S,4aS,8aS)—N-tert-butyl-2-[(2R,3R)-2-hydroxy-3-[(3-hydroxy-2-methylbenzoyl) amino]-4-phenylsulfanylbutyl]-3,4,4a,5,6,7,8,8a-octahydro-1H-isoquinoline-3-carboxamide; Ethyl (E,4S)-4-[[(2R,5S)-2-[(4-fluorophenyl)methyl]-6-methyl-5-[(5-methyl-1,2-oxazole-3-carbonyl)amino]-4-oxoheptanoyl]amino]-5-[(3S)-2-oxopyrrolidin-3-yl]pent-2-enoate; and (R)-3-((2S,3S)-2-Hydroxy-3-{[1-(3-hydroxy-2-methyl-phenyl)-methanoyl]-amino}-4-phenyl-butanoyl)-5,5-dimethyl-thiazolidine-4-carboxylic acid allylamide; or a pharmaceutically acceptable salt thereof; and a pharmaceutically acceptable carrier. E21 is the method of E20 wherein the pharmaceutical composition further comprises an additional therapeutic agent. E22 is the method of E21 wherein the pharmaceutical composition further comprises at least one of a pharmaceutically acceptable interferon, p-glycoprotein inhibitor and CYP3A4 inhibitor.

E23 is a compound selected from the group consisting of: N—((S)-1-(((S)-1-(benzo[d]thiazol-2-yl)-1-oxo-3-((S)-2-oxopyrrolidin-3-yl)propan-2-yl)amino)-3-cyclopropyl-1-oxopropan-2-yl)picolinamide; N—((S)-1-(((S)-1-(benzo[d]thiazol-2-yl)-1-oxo-3-((S)-2-oxopyrrolidin-3-yl)propan-2-yl)amino)-3-cyclopentyl-1-oxopropan-2-yl)-4-methoxy-1H-indole-2-carboxamide; and N—((S)-2-(((S)-1-(benzo[d]thiazol-2-yl)-1-oxo-3-((S)-2-oxopyrrolidin-3-yl)propan-2-yl)amino)-1-cyclopentyl-2-oxoethyl)-4-methoxy-1H-indole-2-carboxamide; or a pharmaceutically acceptable salt thereof.

E24 is the method of E1 further comprising administering to a patient in need thereof a therapeutically effective amount of an additional therapeutic agent selected from one or more of remdesivir, azithromycin, chloroquine and hydroxychloroquine.

E25 is the method of E24 wherein N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide or a pharmaceutically acceptable salt thereof is administered.

E26 is the method of E25 wherein one or both of remdesivir and azithromycin are administered.

E27 is a method of treating a condition that is mediated by SARS-CoV-2 coronavirus 3C-like protease activity in a patient by administering to said patient a pharmaceutically effective amount of a SARS-CoV-2 protease inhibitor as described herein.

E28 is a method of targeting SARS-CoV-2 inhibition as a means of treating indications caused by SARS-CoV-2-related viral infections.

E29 is a method of identifying cellular or viral pathways interfering with the functioning of the members of which could be used for treating indications caused by SARS-CoV-2 infections by administering a SARS-CoV-2 protease inhibitor as described herein.

E30 is a method of using SARS-CoV-2 protease inhibitors as described herein as tools for understanding mechanism of action of other SARS-CoV-2 inhibitors.

E31 is a method of using SARS-CoV-2 3C-like protease inhibitors for carrying out gene profiling experiments for monitoring the up or down regulation of genes for the purposed of identifying inhibitors for treating indications caused by SARS-CoV-2 infections such as COVID-19.

E32 is a pharmaceutical composition for the treatment of COVID-19 in a mammal containing an amount of a SARS-CoV-2 3C-like protease inhibitor that is effective in treating COVID-19 and a pharmaceutically acceptable carrier.

E33 is a method of treating COVID-19 in a patient, the method comprising intravenously administering to a patient in need thereof a therapeutically effective amount of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide.

E34 is the method of E33 wherein 0.2 g/day to 4 g/day of N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide is administered to the patient.

E35 is the method of E34 wherein 0.3 g/day to 3 g/day of N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide is administered to the patient.

E36 is method of any one of E33 through E35 wherein the N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide is administered to the patient by continuous intravenous infusion.

E37 is the method of E36 wherein about 0.3 g/day to 3 g/day of N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide is administered to the patient by continuous intravenous infusion.

E38 is the method of E37 wherein N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide is administered to the patient by continuous intravenous infusion in an amount sufficient to maintain a C_{eff} of approximately 0.5 μM.

E39 is a method of any one of E33 through E38 wherein the N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide is administered in combination with one or more additional therapeutic agents.

E40 is the method of E39 wherein the additional therapeutic agents are remdesivir and azithromycin.

E41 is the method of E40 wherein the remdesivir is co-administered by continuous intravenous infusion.

E42 is the method of E40 wherein azithromycin is co-administered by continuous intravenous infusion.

E43 is the crystalline compound N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide, hydrate (Form 3) having a powder X-ray diffraction pattern comprising three or more X-ray diffraction peaks, in degrees 2-theta, selected from 8.6±0.2, 11.9±0.2, 14.6±0.2, 18.7±0.2 and 19.7±0.2. E44 is the crystalline compound of claim 37 wherein the powder X-ray diffraction peaks, in degrees 2-theta, are 14.6±0.2, 18.7±0.2 and 19.7±0.2. E45 is the crystalline compound of claim 37 wherein the powder X-ray diffraction peaks, in degrees 2-theta, are 8.6±0.2, 14.6±0.2, 18.7±0.2 and 19.7±0.2. E46 is the crystalline compound of claim 37 wherein the powder X-ray diffraction peaks, in degrees 2-theta, are 8.6±0.2, 11.9±0.2, 14.6±0.2, 18.7±0.2 and 19.7±0.2.

E47 is a method of treating COVID-19 in a patient, the method comprising parenterally administering an aqueous liquid composition comprising: a) from about 0.2 mg/mL to about 2.0 mg/mL of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof; b) one or more co-solvents; c) optionally one or more surfactants; and d) a buffer; wherein the final composition has a pH of about 1.5 to about 6 and the total amount of the one or more co-solvents is up to about 30% (v/v).

E48 is the method of E47 wherein the composition is administered intravenously to the patient a volume of about 1000 mL or less per day, has a pH of about 3 to about 5 and the total amount of the one or more co-solvents is up to about 20% (v/v).

E49 is the method of E48 wherein the composition comprises: a) about 0.2 mg/mL to about 1.0 mg/mL of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof; b) the one or more co-solvents are selected from the group consisting of benzyl alcohol (BA), dimethylacrylamide (DMA), dimethyl sulfoxide (DMSO), ethanol, N-methyl pyrrolidone (NMP), polyethylene glycol and propylene glycol (PG), wherein the total amount of the one or more co-solvents is up to about 10% (v/v); c) the one or more surfactants, when present, are selected from the group consisting of polyvinylpyrrolidone (PVP), poloxamer 407, poloxamer 188, hydroxypropyl methylcellulose (HPMC), polyethoxylated castor oil, lecithin, polysorbate 80 (PS80), polysorbate 20 (PS20) and polyethylene glycol (15)-hydroxystearate; and d) the buffer is selected from the group consisting of acetic acid, citric acid, lactic acid, phosphoric acid, and tartaric acid.

E50 is the method of E49 wherein the composition comprises: a) one or two co-solvents selected from the group consisting of dimethyl sulfoxide (DMSO), ethanol, PEG300 and PEG400; b) the one or two surfactants, when present, is selected from the group consisting of polysorbate 80, polysorbate 20 and polyethylene glycol (15)-hydroxystearate; and c) the buffer is citric acid up to 50 mM.

E51 is the method of E50 wherein the composition is administered to the patient by continuous intravenous infusion and the volume of the composition administered to the patient is from about 250 mL to about 500 mL per day.

E52 is a method of treating COVID-19 in a patient, the method comprising parenterally administering an aqueous liquid composition comprising: a) from about 0.2 mg/mL to about 16 mg/mL of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof; b) a complexing agent; c) a buffer; d) optionally one or more co-solvents; and e) optionally one or more surfactants; wherein the final composition has a pH of about 1.5 to about 6, and the total amount of the one or more co-solvents, when present, is up to about 15% (v/v) of the total solution.

E53 is the method of E52 wherein the composition: a) is administered intravenously to the patient a volume of about 1000 mL or less per day; b) has a pH of about 3 to about 5; and c) has a total amount of the one or more co-solvents, when present, up to about 10% (v/v) of the total solution.

E54 is the method of E53 wherein the composition comprises: a) about 0.2 mg/mL to about 8.0 mg/mL of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof; b) a complexing agent selected from the group consisting of α-cyclodextrins, β-cyclodextrins, γ-cyclodextrins, nicotinamide, sodium benzoate and sodium salicylate, where the molar ratio of the complexing agent to N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide is from about 1.5:1 to about 25:1; c) a buffer selected from the group consisting of acetic acid, citric acid, lactic acid, phosphoric acid and tartaric acid; d) one or more co-solvents selected from the group consisting of benzyl alcohol (BA), dimethylacrylamide (DMA), dimethyl sulfoxide (DMSO), ethanol, N-methyl pyrrolidone (NMP), polyethylene glycol and propylene glycol (PG), wherein the total amount of the one or more co-solvents, when present, is up to about 6% (v/v) of the total solution; and e) optionally one or more surfactants selected from the group consisting of polyvinylpyrrolidone (PVP), poloxamer 407, poloxamer 188, hydroxypropyl methylcellulose (HPMC), polyethoxylated castor oil, lecithin, polysorbate 80 (PS80), polysorbate 20 (PS20) and polyethylene glycol (15)-hydroxystearate.

E55 is the method of E54 wherein: a) the complexing agent is a β-cyclodextrin; b) the molar ratio of β-cyclodextrin to N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide is from about 1.5:1 to about 8:1; and c) the buffer is citric acid up to about 50 mM.

E56 is the method of E55 wherein: a) the composition comprises one to two co-solvents selected from the group consisting of dimethyl sulfoxide (DMSO), ethanol, PEG300, PEG400 and propylene glycol (PG); b) the complexing agent is selected from the group consisting of hydroxypropyl-β-cyclodextrin (HP-β-CD) and sulfobutylether-β-cyclodextrin (SBE-β-CD); and c) the surfactant, when present, is selected from polysorbate 80, polysorbate 20 and polyethylene glycol (15)-hydroxystearate.

E57 is the method of E56 wherein: a) the two co-solvents are one of ethanol and dimethyl sulfoxide (DMSO), and the other co-solvent is selected from the group consisting of PEG300, PEG400, and propylene glycol (PG), wherein the ratio of ethanol or DMSO to PEG 300, PEG400 or PG is from about 1:2 to about 1:4; or the two co-solvents are ethanol and DMSO, wherein the ratio of ethanol to DMSO is from about 1:2 to about 1:4; b) the molar ratio of hydroxypropyl-D-cyclodextrin (HP-β-CD) or sulfobutylether-β-cyclodextrin (SBE-β-CD) to N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide is from about 2:1 to about 6:1; and c) the concentration of the citric acid buffer is up to about 50 mM.

E58 is the method of E57 wherein the composition is administered to the patient by continuous intravenous infusion and the volume of the composition administered to the patient is from about 250 mL to about 500 mL per day.

E59 is a pharmaceutical composition comprising: a) a therapeutically effective amount of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof; b) a complexing agent selected from the group consisting of α-cyclodextrins, β-cyclodextrins, γ-cyclodextrins, nicotinamide, sodium benzoate and sodium salicylate, wherein the molar ratio of the complexing agent to N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide is from about 1.5:1 to about 25:1; and c) a buffer.

E60 is the pharmaceutical composition of E59, wherein the pharmaceutical composition is a ready to use or ready to dilute parenteral solution, which: a) optionally comprises one or more co-solvents which are selected from the group consisting of benzyl alcohol (BA), dimethylacrylamide (DMA), dimethyl sulfoxide (DMSO), ethanol, N-methyl pyrrolidone (NMP), polyethylene glycol and propylene glycol (PG); b) further optionally comprises a surfactant which is selected from the group consisting of polyvinylpyrrolidone (PVP), poloxamer 407, poloxamer 188, hydroxypropyl methylcellulose (HPMC), polyethoxylated castor oil, lecithin, polysorbate 80 (PS80), polysorbate 20 (PS20) and polyethylene glycol (15)-hydroxystearate; c) a buffer selected from the group consisting of acetic acid, citric acid, lactic acid, phosphoric acid and tartaric acid; and d) the pH of the parenteral solution is about 3 to about 5.

E61 is the pharmaceutical composition of E60 which comprises: a) N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide or a pharmaceutically acceptable salt thereof at a concentration of about 0.2 mg/mL to about 16 mg/mL; b) the complexing agent is hydroxypropyl-β-cyclodextrin (HP-β-CD) or sulfobutylether-β-cyclodextrin (SBE-β-CD), wherein the molar ratio of the complexing agent to N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide is from about 1.5:1 to about 8:1; c) one or two co-solvents selected from the group consisting of dimethyl sulfoxide (DMSO), ethanol, PEG300, PEG400 and propylene glycol (PG), and the total concentration of the co-solvents is up to about 15% (v/v) of the total solution; d) optionally comprises a surfactant selected from the group consisting of polysorbate 80 (PS80), polysorbate 20 (PS20) and polyethylene glycol (15)-hydroxystearate; and e) the buffer is citric acid.

E62 is the pharmaceutical composition of E61 which comprises: a) N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide or a pharmaceutically acceptable salt thereof at a concentration of about 0.2 mg/mL to about 8 mg/mL; b) the complexing agent is hydroxypropyl-β-cyclodextrin (HP-β-CD) or sulfobutylether-β-cyclodextrin (SBE-β-CD), wherein the molar ratio of the complexing agent to N-((1S)-1-{[((1S)-3-hydroxy-2-oxo-1-{[(3S)-2-oxopyrrolidin-3-yl]methyl}propyl)amino] carbonyl}-3-methylbutyl)-4-methoxy-1H-indole-2-carboxamide is from about 2:1 to about 6:1; and c) one or two co-solvents selected from the group consisting of dimethyl sulfoxide (DMSO), ethanol, PEG300, PEG400 and propylene glycol, and the total concentration of the co-solvents is up to about 10% (v/v) of the total solution.

E63 is the pharmaceutical composition of claim E62 prepared by the following process comprising the steps: a) dissolution of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof in one or more co-solvents selected from the group consisting of dimethyl sulfoxide (DMSO), ethanol, PEG300, PEG400 and propylene glycol (PG) to provide a first non-aqueous solution; b) dissolution of hydroxypropyl-β-cyclodextrin (HP-β-CD) or sulfobutylether-β-cyclodextrin (SBE-β-CD) in a water-containing solution containing a citric acid buffer and optionally a surfactant to provide a second aqueous solution; and c) combining the first non-aqueous solution from step a) with the second aqueous solution from step b) to provide said pharmaceutical composition.

E64 is the pharmaceutical composition of E63 which comprises about 1.1% ethanol (v/v), about 3.4% PEG400 (v/v), about 80 mg/mL SBE-β-cyclodextrin, about 6 mg/mL N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide; up to about 50 mM citric acid and wherein the pH is about 4 to about 5.

E65 is the pharmaceutical composition of E59 wherein the composition is a lyophile or powder ready for reconstitution into a solution suitable for parenteral administration.

E66 is the pharmaceutical composition of E65 wherein the composition a) optionally comprises one or more co-solvents which are selected from the group consisting of benzyl alcohol (BA), dimethylacrylamide (DMA), dimethyl sulfoxide (DMSO), ethanol, N-methyl pyrrolidone (NMP), polyethylene glycol and propylene glycol (PG); b) optionally comprises a surfactant which is selected from the group consisting of polyvinylpyrrolidone (PVP), poloxamer 407, poloxamer 188, hydroxypropyl methylcellulose (HPMC), polyethoxylated castor oil, lecithin, polysorbate 80 (PS80), polysorbate 20 (PS20) and polyethylene glycol (15)-hydroxystearate; and c) comprises a buffer selected from the group consisting of acetic acid, citric acid, lactic acid, phosphoric acid and tartaric acid.

E67 is the pharmaceutical composition of E65 which is a powder ready for reconstitution into a parenteral solution wherein the powder comprises N-((1S)-1-{[((1S)-3-hydroxy-2-oxo-1-{[(3S)-2-oxopyrrolidin-3-yl]methyl}propyl)amino] carbonyl}-3-methylbutyl)-4-methoxy-1H-indole-2-carboxamide, hydrate (Form 3).

E68 is the method of any one of E1 and E47 through E58 wherein one or more additional agent is administered to the patient, wherein the one or more additional agent is selected from antivirals such as remdesivir, galidesivir, favilavir/avifavir, mulnupiravir (MK-4482/EIDD 2801), AT-527, AT-301, BLD-2660, favipiravir, camostat, SLV213 emtrictabine/tenofivir, clevudine, dalcetrapib, boceprevir and ABX464, glucocorticoids such as dexamethasone and hydrocortisone, convalescent plasma, a recombinant human plasma such as gelsolin (Rhu-p65N), monoclonal antibodies such as regdanvimab (Regkirova), ravulizumab (Ultomiris), VIR-7831/VIR-7832, BRII-196/BRII-198, COVI-AMG/COVI DROPS (STI-2020), bamlanivimab (LY-CoV555), mavrilimab, leronlimab (PRO140), AZD7442, lenzilumab, infliximab, adalimumab, JS 016, STI-1499 (COVIGUARD), lanadelumab (Takhzyro), canakinumab (Ilaris), gimsilumab and otilimab, antibody cocktails such as casirivimab/imdevimab (REGN-Cov2), recombinant fusion protein such as MK-7110 (CD24Fc/SACCOVID), anticoagulants such as heparin and apixaban, IL-6 receptor agonists such as tocilizumab (Actemra) and sarilumab (Kevzara), PlKfyve inhibitors such as apilimod dimesylate, RIPK1 inhibitors such as DNL758, VIP receptor agonists such as PB1046, SGLT2 inhibitors such as dapaglifozin, TYK inhibitors such as abivertinib, kinase inhibitors such as ATR-002, bemcentinib, acalabrutinib and losmapimod, H2 blockers such as famotidine, anthelmintics such as niclosamide, furin inhibitors such as diminazene.

It is to be understood that the method of treatment embodiments of the invention can also to be construed as medical use-type embodiments (such as E1A below) or alternatively second medical use-type embodiments (such as E1B below).

E1A is a compound selected from the group consisting of: (3S)-3-({4-methyl-N-[(2R)-tetrahydrofuran-2-ylcarbonyl]-L-leucyl}amino)-2-oxo-4-[(3S)-2-oxopyrrolidin-3-yl]butyl 2,6-dichlorobenzoate; (3S)-3-({N-[(4-methoxy-1H-indol-2-yl)carbonyl]-L-leucyl}amino)-2-oxo-4-[(3S)-2-oxopyrrolidin-3-yl]butyl cyclopropanecarboxylate; N—((S)-1-(((S)-1-(benzo[d]thiazol-2-yl)-1-oxo-3-((S)-2-oxopyrrolidin-3-yl)propan-2-yl)amino)-3-cyclopropyl-1-oxopropan-2-yl)picolinamide; N—((S)-1-(((S)-1-(benzo[d]thiazol-2-yl)-1-oxo-3-((S)-2-oxopyrrolidin-3-yl)propan-2-yl)amino)-3-cyclopentyl-1-oxopropan-2-yl)-4-methoxy-1H-indole-2-carboxamide; N—((S)-2-(((S)-1-(benzo[d]thiazol-2-yl)-1-oxo-3-((S)-2-oxopyrrolidin-3-yl)propan-2-yl)amino)-1-cyclopentyl-2-oxoethyl)-4-methoxy-1H-indole-2-carboxamide; N-((1S)-1-{[((1S)-3-hydroxy-2-oxo-1-{[(3S)-2-oxopyrrolidin-3-yl]methyl}propyl)amino]carbonyl}-3,3-dimethylbutyl)-1H-indole-2-carboxamide; N-((1S)-1-{[((1S)-3-hydroxy-2-oxo-1-{[(3S)-2-oxopyrrolidin-3-yl]methyl} propyl)amino]carbonyl}pentyl)-4-methoxy-1H-indole-2-carboxamide; N—((S)-1-(((S)-4-hydroxy-3-oxo-1-((S)-2-oxopyrrolidin-3-yl)butan-2-yl)amino)-1-oxo-3-phenylpropan-2-yl)-4-methoxy-1H-indole-2-carboxamide; N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide; (2R)-2-cyclopentyl-2-[2-(2,6-diethylpyridin-4-yl)ethyl]-5-[(5,7-dimethyl-[1,2,4]triazolo[1,5-a]pyrimidin-2-yl)methyl]-4-hydroxy-3H-pyran-6-one; (3S,4aS,8aS)—N-tert-butyl-2-[(2R,3R)-2-hydroxy-3-[(3-hydroxy-2-methylbenzoyl) amino]-4-phenylsulfanylbutyl]-3,4,4a,5,6,7,8,8a-octahydro-1H-isoquinoline-3-carboxamide; Ethyl (E,4S)-4-[[(2R,5S)-2-[(4-fluorophenyl)methyl]-6-methyl-5-[(5-methyl-1,2-oxazole-3-carbonyl)amino]-4-oxoheptanoyl]amino]-5-[(3S)-2-oxopyrrolidin-3-yl]pent-2-enoate; and (R)-3-((2S,3S)-2-Hydroxy-3-{[1-(3-hydroxy-2-methyl-phenyl)-methanoyl]-amino}-4-phenyl-butanoyl)-5,5-dimethyl-thiazolidine-4-carboxylic acid allylamide; or a pharmaceutically acceptable salt thereof for use in the treatment of COVID-19 in a patient.

E1B is a compound selected from the group consisting of: (3S)-3-({4-methyl-N-[(2R)-tetrahydrofuran-2-ylcarbonyl]-L-leucyl}amino)-2-oxo-4-[(3S)-2-oxopyrrolidin-3-yl]butyl 2,6-dichlorobenzoate; (3S)-3-({N-[(4-methoxy-1H-indol-2-yl)carbonyl]-L-leucyl}amino)-2-oxo-4-[(3S)-2-oxopyrrolidin-3-yl]butyl cyclopropanecarboxylate; N—((S)-1-(((S)-1-(benzo[d]thiazol-2-yl)-1-oxo-3-((S)-2-oxopyrrolidin-3-yl)propan-2-yl)amino)-3-cyclopropyl-1-oxopropan-2-yl)picolinamide; N—((S)-1-(((S)-1-(benzo[d]thiazol-2-yl)-1-oxo-3-((S)-2-oxopyrrolidin-3-yl)propan-2-yl)amino)-3-cyclopentyl-1-oxopropan-2-yl)-4-methoxy-1H-indole-2-carboxamide; N—((S)-2-(((S)-1-(benzo[d]thiazol-2-yl)-1-oxo-3-((S)-2-oxopyrrolidin-3-yl)propan-2-yl)amino)-1-cyclopentyl-2-oxoethyl)-4-methoxy-1H-indole-2-carboxamide; N-((1S)-1-{[((1S)-3-hydroxy-2-oxo-1-{[(3S)-2-oxopyrrolidin-3-yl]methyl}propyl)amino]carbonyl}-3,3-dimethylbutyl)-1H-indole-2-carboxamide; N-((1S)-1-{[((1S)-3-hydroxy-2-oxo-1-{[(3S)-2-oxopyrrolidin-3-yl]methyl} propyl)amino]carbonyl}pentyl)-4-methoxy-1H-indole-2-carboxamide; N—((S)-1-(((S)-4-hydroxy-3-oxo-1-((S)-2-oxopyrrolidin-3-yl)butan-2-yl)amino)-1-oxo-3-phenylpropan-2-yl)-4-methoxy-1H-indole-2-carboxamide; N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide; (2R)-2-cyclopentyl-2-[2-(2,6-diethylpyridin-4-yl)ethyl]-5-[(5,7-dimethyl-[1,2,4]triazolo[1,5-a]pyrimidin-2-yl)methyl]-4-hydroxy-3H-pyran-6-one; (3S,4aS,8aS)—N-tert-butyl-2-[(2R,3R)-2-hydroxy-3-[(3-hydroxy-2-methylbenzoyl) amino]-4-phenylsulfanylbutyl]-3,4,4a,5,6,7,8,8a-octahydro-1H-isoquinoline-3-carboxamide; Ethyl (E,4S)-4-[[(2R,5S)-2-[(4-fluorophenyl)methyl]-6-methyl-5-[(5-methyl-1,2-oxazole-3-carbonyl)amino]-4-oxoheptanoyl]amino]-5-[(3S)-2-oxopyrrolidin-3-yl]pent-2-enoate; and (R)-3-((2S,3S)-2-Hydroxy-3-{[1-(3-hydroxy-2-methyl-phenyl)-methanoyl]-amino}-4-phenyl-butanoyl)-5,5-dimethyl-thiazolidine-4-carboxylic acid allylamide; or a pharmaceutically acceptable salt thereof for use in the preparation of a medicament for the treatment of COVID-19 in a patient.

E2A is the compound for use according to E1A wherein the compound is administered orally or intravenously. E3A is the compound for use according to E2A wherein the compound is administered intravenously. E4A is the compound for use according to E3A wherein the compound is administered intermittently over a 24-hour period or continuously over a 24-hour period. E5A is the compound for use according to any one of E1A to E4A wherein the compound is (3S)-3-({4-methyl-N-[(2R)-tetrahydrofuran-2-ylcarbonyl]-L-leucyl}amino)-2-oxo-4-[(3S)-2-oxopyrrolidin-3-yl]butyl 2,6-dichlorobenzoate; or a pharmaceutically acceptable salt thereof. E6A is the compound for use according to any one of E1A to E4A wherein the compound is (3S)-3-({N-[(4-methoxy-1H-indol-2-yl)carbonyl]-L-leucyl}amino)-2-oxo-4-[(3S)-2-oxopyrrolidin-3-yl]butyl cyclopropane carboxylate; or a pharmaceutically acceptable salt thereof. E7A is the compound for use according to any one of E1A to E4A wherein the compound is N—((S)-1-(((S)-1-(benzo[d]thiazol-2-yl)-1-oxo-3-((S)-2-oxopyrrolidin-3-yl)propan-2-yl)amino)-3-cyclopropyl-1-oxopropan-2-yl)picolinamide; or a pharmaceutically acceptable salt thereof. E8A is the compound for use according to any one of E1A to E4A wherein the compound is N—((S)-1-(((S)-1-(benzo[d]thiazol-2-yl)-1-oxo-3-((S)-2-oxopyrrolidin-3-yl)propan-2-yl)amino)-3-cyclopentyl-1-oxopropan-2-yl)-4-methoxy-1H-indole-2-carboxamide; or a pharmaceutically acceptable salt thereof. E9A is the compound for use according to any one of E1A to E4A wherein the compound is N—((S)-2-(((S)-1-(benzo[d]thiazol-2-yl)-1-oxo-3-((S)-2-oxopyrrolidin-3-yl)propan-2-yl)amino)-1-cyclopentyl-2-oxoethyl)-4-methoxy-1H-indole-2-carboxamide; or a pharmaceutically acceptable salt thereof. E10A is the compound for use according to any one of E1A to E4A wherein the compound is N-((1S)-1-{[((1S)-3-hydroxy-2-oxo-1-{[(3S)-2-oxopyrrolidin-3-yl]methyl}propyl)amino] carbonyl}-3,3-dimethylbutyl)-1H-indole-2-carboxamide; or a pharmaceutically acceptable salt thereof. E11A is the compound for use according to any one of E1A to E4A wherein the compound is N-((1S)-1-{[((1S)-3-hydroxy-2-oxo-1-{[(3S)-2-oxopyrrolidin-3-yl]methyl} propyl)amino] carbonyl}pentyl)-4-methoxy-1H-indole-2-carboxamide; or a pharmaceutically acceptable salt thereof.

E12A is the compound for use according to any one of E1A to E4A wherein the compound is N—((S)-1-(((S)-4-hydroxy-3-oxo-1-((S)-2-oxopyrrolidin-3-yl)butan-2-yl)amino)-1-oxo-3-phenylpropan-2-yl)-4-methoxy-1H-indole-2-carboxamide; or a pharmaceutically acceptable salt thereof. E13A is the compound for use according to any one of E1A to E4A wherein the compound is N-((1S)-1-{[((1S)-3-hydroxy-2-oxo-1-{[(3S)-2-oxopyrrolidin-3-yl]methyl}propyl)amino] carbonyl}-3-methylbutyl)-4-methoxy-1H-indole-2-carboxamide; or a pharmaceutically acceptable salt thereof. E14A is the compound for use according to any one of E1A to E4A wherein the compound is (2R)-2-cyclopentyl-2-[2-(2,6-diethylyridin-4-yl)ethyl]-5-[(5,7-dimethyl-[1,2,4] triazolo[1,5-a]pyrimidin-2-yl)methyl]-4-hydroxy-3H-pyran-6-one; or a pharmaceutically acceptable salt thereof. E15A is the compound for use according to any one of E1A to E4A wherein the compound is (3S,4aS,8aS)—N-tert-butyl-2-[(2R,3R)-2-hydroxy-3-[(3-hydroxy-2-methylbenzoyl) amino]-4-phenylsulfanylbutyl]-3,4,4a,5,6,7,8,8a-octahydro-1H-isoquinoline-3-carboxamide; or a pharmaceutically acceptable salt thereof. E16A is the compound for use according to any one of E1A to E4A wherein the compound is Ethyl (E,4S)-4-[[(2R,5S)-2-[(4-fluorophenyl)methyl]-6-methyl-5-[(5-methyl-1,2-oxazole-3-carbonyl)amino]-4-oxoheptanoyl]amino]-5-[(3S)-2-oxopyrrolidin-3-yl]pent-2-enoate; or a pharmaceutically acceptable salt thereof. E17A is the compound for use according to any one of E1A to E4A wherein the compound is (R)-3-((2S,3S)-2-Hydroxy-3-{[1-(3-hydroxy-2-methyl-phenyl)-methanoyl]-amino}-4-phenyl-butanoyl)-5,5-dimethyl-thiazolidine-4-carboxylic acid allylamide; or a pharmaceutically acceptable salt thereof.

E18A is a compound selected from the group consisting of: (3S)-3-({4-methyl-N-[(2R)-tetrahydrofuran-2-ylcarbonyl]-L-leucyl}amino)-2-oxo-4-[(3S)-2-oxopyrrolidin-3-yl]butyl 2,6-dichlorobenzoate; (3S)-3-({N-[(4-methoxy-1H-indol-2-yl)carbonyl]-L-leucyl}amino)-2-oxo-4-[(3S)-2-oxopyrrolidin-3-yl]butyl cyclopropanecarboxylate; N—((S)-1-(((S)-1-(benzo[d]thiazol-2-yl)-1-oxo-3-((S)-2-oxopyrrolidin-3-yl)propan-2-yl)amino)-3-cyclopropyl-1-oxopropan-2-yl)picolinamide; N—((S)-1-(((S)-1-(benzo[d]thiazol-2-yl)-1-oxo-3-((S)-2-oxopyrrolidin-3-yl)propan-2-yl)amino)-3-cyclopentyl-1-oxopropan-2-yl)-4-methoxy-1H-indole-2-carboxamide; N—((S)-2-(((S)-1-(benzo[d]thiazol-2-yl)-1-oxo-3-((S)-2-oxopyrrolidin-3-yl)propan-2-yl)amino)-1-cyclopentyl-2-oxoethyl)-4-methoxy-1H-indole-2-carboxamide; N-((1S)-1-{[((1S)-3-hydroxy-2-oxo-1-{[(3S)-2-oxopyrrolidin-3-yl]methyl}propyl)amino]carbonyl}-3,3-dimethylbutyl)-1H-indole-2-carboxamide; N-((1S)-1-{[((1S)-3-hydroxy-2-oxo-1-{[(3S)-2-oxopyrrolidin-3-yl]methyl} propyl)amino]carbonyl}pentyl)-4-methoxy-1H-indole-2-carboxamide; N—((S)-1-(((S)-4-hydroxy-3-oxo-1-((S)-2-oxopyrrolidin-3-yl)butan-2-yl)amino)-1-oxo-3-phenylpropan-2-yl)-4-methoxy-1H-indole-2-carboxamide; N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide; (2R)-2-cyclopentyl-2-[2-(2,6-diethylpyridin-4-yl)ethyl]-5-[(5,7-dimethyl-[1,2,4]triazolo[1,5-a]pyrimidin-2-yl)methyl]-4-hydroxy-3H-pyran-6-one; (3S,4aS,8aS)—N-tert-butyl-2-[(2R,3R)-2-hydroxy-3-[(3-hydroxy-2-methylbenzoyl) amino]-4-phenylsulfanylbutyl]-3,4,4a,5,6,7,8,8a-octahydro-1H-isoquinoline-3-carboxamide; Ethyl (E,4S)-4-[[(2R,5S)-2-[(4-fluorophenyl)methyl]-6-methyl-5-[(5-methyl-1,2-oxazole-3-carbonyl)amino]-4-oxoheptanoyl]amino]-5-[(3S)-2-oxopyrrolidin-3-yl]pent-2-enoate; and (R)-3-((2S,3S)-2-Hydroxy-3-{[1-(3-hydroxy-2-methyl-phenyl)-methanoyl]-amino}-4-phenyl-butanoyl)-5,5-dimethyl-thiazolidine-4-carboxylic acid allylamide; or a pharmaceutically acceptable salt thereof for use in the inhibition or prevention of SARS-CoV-2 viral replication in a patient.

E19A is a compound selected from the group consisting of: (3S)-3-({4-methyl-N-[(2R)-tetrahydrofuran-2-ylcarbonyl]-L-leucyl}amino)-2-oxo-4-[(3S)-2-oxopyrrolidin-3-yl]butyl 2,6-dichlorobenzoate; (3S)-3-({N-[(4-methoxy-1H-indol-2-yl)carbonyl]-L-leucyl}amino)-2-oxo-4-[(3S)-2-oxopyrrolidin-3-yl]butyl cyclopropane carboxylate; N—((S)-1-(((S)-1-(benzo[d]thiazol-2-yl)-1-oxo-3-((S)-2-oxopyrrolidin-3-yl)propan-2-yl)amino)-3-cyclopropyl-1-oxopropan-2-yl)picolinamide; N—((S)-1-(((S)-1-(benzo[d]thiazol-2-yl)-1-oxo-3-((S)-2-oxopyrrolidin-3-yl)propan-2-yl)amino)-3-cyclopentyl-1-oxopropan-2-yl)-4-methoxy-1H-indole-2-carboxamide; N—((S)-2-(((S)-1-(benzo[d]thiazol-2-yl)-1-oxo-3-((S)-2-oxopyrrolidin-3-yl)propan-2-yl)amino)-1-cyclopentyl-2-oxoethyl)-4-methoxy-1H-indole-2-carboxamide; N-((1S)-1-{[((1S)-3-hydroxy-2-oxo-1-{[(3S)-2-oxopyrrolidin-3-yl]methyl}propyl)amino]carbonyl}-3,3-dimethylbutyl)-1H-indole-2-carboxamide; N-((1S)-1-{[((1S)-3-hydroxy-2-oxo-1-{[(3S)-2-oxopyrrolidin-3-yl]methyl} propyl)amino]carbonyl}pentyl)-4-methoxy-1H-indole-2-carboxamide; N—((S)-1-(((S)-4-hydroxy-3-oxo-1-((S)-2-oxopyrrolidin-3-yl)butan-2-yl)amino)-1-oxo-3-phenylpropan-2-yl)-4-methoxy-1H-indole-2-carboxamide; and N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide; or a pharmaceutically acceptable salt thereof for for the use of inhibition or prevention of SARS-CoV-2 viral replication.

E20A is a pharmaceutical composition, the pharmaceutical composition comprising a therapeutically effective amount of a compound selected from the group consisting of: (3S)-3-({4-methyl-N-[(2R)-tetrahydrofuran-2-ylcarbonyl]-L-leucyl}amino)-2-oxo-4-[(3S)-2-oxopyrrolidin-3-yl]butyl 2,6-dichlorobenzoate; (3S)-3-({N-[(4-methoxy-1H-indol-2-yl)carbonyl]-L-leucyl}amino)-2-oxo-4-[(3S)-2-oxopyrrolidin-3-yl]butyl cyclopropanecarboxylate; N—((S)-1-(((S)-1-(benzo[d]thiazol-2-yl)-1-oxo-3-((S)-2-oxopyrrolidin-3-yl)propan-2-yl)amino)-3-cyclopropyl-1-oxopropan-2-yl)picolinamide; N—((S)-1-(((S)-1-(benzo[d]thiazol-2-yl)-1-oxo-3-((S)-2-oxopyrrolidin-3-yl)propan-2-yl)amino)-3-cyclopentyl-1-oxopropan-2-yl)-4-methoxy-1H-indole-2-carboxamide; N—((S)-2-(((S)-1-(benzo[d]thiazol-2-yl)-1-oxo-3-((S)-2-oxopyrrolidin-3-yl)propan-2-yl)amino)-1-cyclopentyl-2-oxoethyl)-4-methoxy-1H-indole-2-carboxamide; N-((1S)-1-{[((1S)-3-hydroxy-2-oxo-1-{[(3S)-2-oxopyrrolidin-3-yl]methyl}propyl) amino]carbonyl}-3,3-dimethylbutyl)-1H-indole-2-carboxamide; N-((1S)-1-{[((1S)-3-hydroxy-2-oxo-1-{[(3S)-2-oxopyrrolidin-3-yl]methyl} propyl)amino] carbonyl}pentyl)-4-methoxy-1H-indole-2-carboxamide; N—((S)-1-(((S)-4-hydroxy-3-oxo-1-((S)-2-oxopyrrolidin-3-yl)butan-2-yl)amino)-1-oxo-3-phenylpropan-2-yl)-4-methoxy-1H-indole-2-carboxamide; N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide; (2R)-2-cyclopentyl-2-[2-(2,6-diethylpyridin-4-yl)ethyl]-5-[(5,7-dimethyl-[1,2,4] triazolo[1,5-a]pyrimidin-2-yl)methyl]-4-hydroxy-3H-pyran-6-one; (3S,4aS,8aS)—N-tert-butyl-2-[(2R,3R)-2-hydroxy-3-[(3-hydroxy-2-methylbenzoyl) amino]-4-phenylsulfanylbutyl]-3,4,4a,5,6,7,8,8a-octahydro-1H-isoquinoline-3-carboxamide; Ethyl (E,4S)-4-[[(2R,5S)-2-[(4-fluorophenyl)methyl]-6-methyl-5-[(5-methyl-1,2-oxazole-3-carbonyl)amino]-4-oxoheptanoyl]amino]-5-[(3S)-2-oxopyrrolidin-3-yl]pent-2-enoate; and (R)-3-((2S,3S)-2-Hydroxy-3-{[1-(3-hydroxy-2-methyl-phenyl)-methanoyl]-amino}-4-phenyl-butanoyl)-5,5-dimethyl-thiazolidine-4-carboxylic acid allylamide; or a pharmaceutically acceptable salt thereof; and a pharmaceutically acceptable carrier for use in the treatment of COVID-19 in a patient.

E21A is the composition for use according to E20A wherein the pharmaceutical composition further comprises an additional therapeutic agent. E22A is the composition for use according to E20A wherein the pharmaceutical composition further comprises at least one of a pharmaceutically acceptable interferon, p-glycoprotein inhibitor and CYP3A4 inhibitor.

E24A is the composition for use according to E1A further comprising an additional therapeutic agent selected from one or more of remdesivir, azithromycin, chloroquine and hydroxychloroquine for use in the treatment of COVID-19 in a patient.

E25A is the composition for use according to E24A wherein the compound is N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof.

E26A is the composition for use according to E25A wherein one or both of remdesivir and azithromycin are the additional therapeutic agents.

E47A is an aqueous liquid composition comprising: a) from about 0.2 mg/mL to about 2.0 mg/mL of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof; b) one or more co-solvents; c) optionally one or more surfactants; and d) a buffer; wherein the final composition has a pH of about 1.5 to about 6 and the total amount of the one or more co-solvents is up to about 30% (v/v) for use in the treatment of COVID-19 in a patient.

E48A is the composition for use according to E47A wherein the composition is administered intravenously to the patient a volume of about 1000 mL or less per day, has a pH of about 3 to about 5 and the total amount of the one or more co-solvents is up to about 20% (v/v).

E49A is the composition for use according to E48A wherein the composition comprises: a) about 0.2 mg/mL to about 1.0 mg/mL of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof; b) the one or more co-solvents are selected from the group consisting of benzyl alcohol (BA), dimethylacrylamide (DMA), dimethyl sulfoxide (DMSO), ethanol, N-methyl pyrrolidone (NMP), polyethylene glycol and propylene glycol (PG), wherein the total amount of the one or more co-solvents is up to about 10% (v/v); c) the one or more surfactants, when present, are selected from the group consisting of polyvinylpyrrolidone (PVP), poloxamer 407, poloxamer 188, hydroxypropyl methylcellulose (HPMC), polyethoxylated castor oil, lecithin, polysorbate 80 (PS80), polysorbate 20 (PS20) and polyethylene glycol (15)-hydroxystearate; and d) the buffer is selected from the group consisting of acetic acid, citric acid, lactic acid, phosphoric acid, and tartaric acid.

E50A is the composition for use according to E49A wherein the composition comprises: a) one or two co-solvents selected from the group consisting of dimethyl sulfoxide (DMSO), ethanol, PEG300 and PEG400; b) the one or two surfactants, when present, is selected from the group consisting of polysorbate 80, polysorbate 20 and polyethylene glycol (15)-hydroxystearate; and c) the buffer is citric acid up to 50 mM.

E51A is the composition for use according to E50A wherein the composition is administered to the patient by continuous intravenous infusion and the volume of the composition administered to the patient is from about 250 mL to about 500 mL per day.

E52A is an aqueous liquid composition comprising: a) from about 0.2 mg/mL to about 16 mg/mL of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof; b) a complexing agent; c) a buffer; d) optionally one or more co-solvents; and e) optionally one or more surfactants; wherein the final composition has a pH of about 1.5 to about 6, and the total amount of the one or more co-solvents, when present, is up to about 15% (v/v) of the total solution for use in the treatment of COVID-19 in a patient.

E53A is the composition for use according to E52A wherein the composition: a) is administered intravenously to the patient a volume of about 1000 mL or less per day; b) has a pH of about 3 to about 5; and c) has a total amount of the one or more co-solvents, when present, up to about 10% (v/v) of the total solution.

E54A is the composition for use according to E53A wherein the composition comprises: a) about 0.2 mg/mL to about 8.0 mg/mL of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof; b) a complexing agent selected from the group consisting of α-cyclodextrins, β-cyclodextrins, γ-cyclodextrins, nicotinamide, sodium benzoate and sodium salicylate, where the molar ratio of the complexing agent to N-((1S)-1-{[((1S)-3-hydroxy-2-oxo-1-{[(3S)-2-oxopyrrolidin-3-yl]methyl}propyl)amino] carbonyl}-3-methylbutyl)-4-methoxy-1H-indole-2-carboxamide is from about 1.5:1 to about 25:1; c) a buffer selected from the group consisting of acetic acid, citric acid, lactic acid, phosphoric acid and tartaric acid; d) one or more co-solvents selected from the group consisting of benzyl alcohol (BA), dimethylacrylamide (DMA), dimethyl sulfoxide (DMSO), ethanol, N-methyl pyrrolidone (NMP), polyethylene glycol and propylene glycol (PG), wherein the total amount of the one or more co-solvents, when present, is up to about 6% (v/v) of the total solution; and e) optionally one or more surfactants selected from the group consisting of polyvinylpyrrolidone (PVP), poloxamer 407, poloxamer 188, hydroxypropyl methylcellulose (HPMC), polyethoxylated castor oil, lecithin, polysorbate 80 (PS80), polysorbate 20 (PS20) and polyethylene glycol (15)-hydroxystearate.

E55A is the composition for use according to E54A wherein: a) the complexing agent is a β-cyclodextrin; b) the molar ratio of β-cyclodextrin to N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide is from about 1.5:1 to about 8:1; and c) the buffer is citric acid up to about 50 mM.

E56A is the composition for use according to E55A wherein: a) the composition comprises one to two co-solvents selected from the group consisting of dimethyl sulfoxide (DMSO), ethanol, PEG300, PEG400 and propylene glycol (PG); b) the complexing agent is selected from the group consisting of hydroxypropyl-β-cyclodextrin (HP-β-CD) and sulfobutylether-β-cyclodextrin (SBE-β-CD); and c) the surfactant, when present, is selected from polysorbate 80, polysorbate 20 and polyethylene glycol (15)-hydroxystearate.

E57A is the composition for use according to E56A wherein: a) the two co-solvents are one of ethanol and dimethyl sulfoxide (DMSO), and the other co-solvent is selected from the group consisting of PEG300, PEG400, and propylene glycol (PG), wherein the ratio of ethanol or DMSO to PEG 300, PEG400 or PG is from about 1:2 to about 1:4; or the two co-solvents are ethanol and DMSO, wherein the ratio of ethanol to DMSO is from about 1:2 to about 1:4; b) the molar ratio of hydroxypropyl-β-cyclodextrin (HP-3-CD) or sulfobutylether-β-cyclodextrin (SBE-β-CD) to N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide is from about 2:1 to about 6:1; and c) the concentration of the citric acid buffer is up to about 50 mM.

E58A is the composition for use according to E57A wherein the composition is administered to the patient by continuous intravenous infusion and the volume of the composition administered to the patient is from about 250 mL to about 500 mL per day.

E59A is a pharmaceutical composition comprising: a) a therapeutically effective amount of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof; b) a complexing agent selected from the group consisting of α-cyclodextrins, β-cyclodextrins, γ-cyclodextrins, nicotinamide, sodium benzoate and sodium salicylate, wherein the molar ratio of the complexing agent to N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide is from about 1.5:1 to about 25:1; and c) a buffer for use in the treatment of COVID-19 in a patient.

E60A is the composition for use according to E59A, wherein the pharmaceutical composition is a ready to use or ready to dilute parenteral solution, which: a) optionally comprises one or more co-solvents which are selected from the group consisting of benzyl alcohol (BA), dimethylacrylamide (DMA), dimethyl sulfoxide (DMSO), ethanol, N-methyl pyrrolidone (NMP), polyethylene glycol and propylene glycol (PG); b) further optionally comprises a surfactant which is selected from the group consisting of polyvinylpyrrolidone (PVP), poloxamer 407, poloxamer 188, hydroxypropyl methylcellulose (HPMC), polyethoxylated castor oil, lecithin, polysorbate 80 (PS80), polysorbate 20 (PS20) and polyethylene glycol (15)-hydroxystearate; c) a buffer selected from the group consisting of acetic acid, citric acid, lactic acid, phosphoric acid and tartaric acid; and d) the pH of the parenteral solution is about 3 to about 5.

E61A is the composition for use according to E60A which comprises: a) N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide or a pharmaceutically acceptable salt thereof at a concentration of about 0.2 mg/mL to about 16 mg/mL; b) the complexing agent is hydroxypropyl-β-cyclodextrin (HP-β-CD) or sulfobutylether-β-cyclodextrin (SBE-β-CD), wherein the molar ratio of the complexing agent to N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide is from about 1.5:1 to about 8:1; c) one or two co-solvents selected from the group consisting of dimethyl sulfoxide (DMSO), ethanol, PEG300, PEG400 and propylene glycol (PG), and the total concentration of the co-solvents is up to about 15% (v/v) of the total solution; d) optionally comprises a surfactant selected from the group consisting of polysorbate 80 (PS80), polysorbate 20 (PS20) and polyethylene glycol (15)-hydroxystearate; and e) the buffer is citric acid.

E62A is the composition for use according to E61A which comprises: a) N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide or a pharmaceutically acceptable salt thereof at a concentration of about 0.2 mg/mL to about 8 mg/mL; b) the complexing agent is hydroxypropyl-β-cyclodextrin (HP-β-CD) or sulfobutylether-β-cyclodextrin (SBE-β-CD), wherein the molar ratio of the complexing agent to N-((1S)-1-{[((1S)-3-hydroxy-2-oxo-1-{[(3S)-2-oxopyrrolidin-3-yl]methyl}propyl)amino] carbonyl}-3-methylbutyl)-4-methoxy-1H-indole-2-carboxamide is from about 2:1 to about 6:1; and c) one or two co-solvents selected from the group consisting of dimethyl sulfoxide (DMSO), ethanol, PEG300, PEG400 and propylene glycol, and the total concentration of the co-solvents is up to about 10% (v/v) of the total solution.

E63A is the composition for use according to E62A wherein the pharmaceutical composition is prepared by the following process comprising the steps: a) dissolution of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof in one or more co-solvents selected from the group consisting of dimethyl sulfoxide (DMSO), ethanol, PEG300, PEG400 and propylene glycol (PG) to provide a first non-aqueous solution; b) dissolution of hydroxypropyl-β-cyclodextrin (HP-β-CD) or sulfobutylether-β-cyclodextrin (SBE-β-CD) in a water-containing solution containing a buffer and optionally a surfactant to provide a second aqueous solution; and c) combining the first non-aqueous solution from step a) with the second aqueous solution from step b) to provide said pharmaceutical composition.

E64A is the composition for use according to E63A in which the pharmaceutical composition comprises about 1.1% ethanol (v/v), about 3.4% PEG400 (v/v), about 80 mg/mL SBE-β-cyclodextrin, about 6 mg/mL N-((1S)-1-{[((1S)-3-hydroxy-2-oxo-1-{[(3S)-2-oxopyrrolidin-3-yl]methyl} propyl)amino] carbonyl}-3-methylbutyl)-4-methoxy-1H-indole-2-carboxamide; up to about 50 mM citric acid and wherein the pH is about 4 to about 5.

E65A is the composition for use according to E59A wherein the composition is a lyophile or powder ready for reconstitution into a solution suitable for use in the treatment of COVID-19 in a patient by parenteral administration.

E66A is the composition for use according to E65A wherein the composition a) optionally comprises one or more co-solvents which are selected from the group consisting of benzyl alcohol (BA), dimethylacrylamide (DMA), dimethyl sulfoxide (DMSO), ethanol, N-methyl pyrrolidone (NMP), polyethylene glycol and propylene glycol (PG); b) optionally comprises a surfactant which is selected from the group consisting of polyvinylpyrrolidone (PVP), poloxamer 407, poloxamer 188, hydroxypropyl methylcellulose (HPMC), polyethoxylated castor oil, lecithin, polysorbate 80 (PS80), polysorbate 20 (PS20) and polyethylene glycol (15)-hydroxystearate; and c) comprises a buffer selected from the group consisting of acetic acid, citric acid, lactic acid, phosphoric acid and tartaric acid.

E67A is the composition for use according to E65A wherein the pharmaceutical composition is a powder ready for reconstitution into a parenteral solution wherein the powder comprises N-((1S)-1-{[((1S)-3-hydroxy-2-oxo-1-{[(3S)-2-oxopyrrolidin-3-yl]methyl}propyl)amino] carbonyl}-3-methylbutyl)-4-methoxy-1H-indole-2-carboxamide, hydrate (Form 3).

E68A is the composition for use according to any one of E1A through E26A and E47A through E67 wherein one or more additional agents selected from the group consisting of remdesivir, galidesivir, favilavir/avifavir, mulnupiravir (MK-4482/EIDD 2801), AT-527, AT-301, BLD-2660, favipiravir, camostat, SLV213 emtrictabine/tenofivir, clevudine, dalcetrapib, boceprevir, ABX464, dexamethasone, hydrocortisone, convalescent plasma, gelsolin (Rhu-p65N), regdanvimab (Regkirova), ravulizumab (Ultomiris), VIR-7831/VIR-7832, BRII-196/BRII-198, COVI-AMG/COVI DROPS (STI-2020), bamlanivimab (LY-CoV555), mavrilimab, leronlimab (PRO140), AZD7442, lenzilumab, infliximab, adalimumab, JS 016, STI-1499 (COVIGUARD), lanadelumab (Takhzyro), canakinumab (Ilaris), gimsilumab, otilimab, casirivimab/imdevimab (REGN-Cov2), MK-7110 (CD24Fc/SACCOVID), heparin, apixaban, tocilizumab (Actemra), sarilumab (Kevzara), apilimod dimesylate, DNL758, PB1046, dapaglifozin, abivertinib, ATR-002, bemcentinib, acalabrutinib, losmapimod, famotidine, niclosamide and diminazene are used for the treatment of COVID-19 in a patient.

### BRIEF DESCRIPTION OF THE DRAWINGS

FIG. 1: Depiction of the residue differences between SARS-CoV and SARS-CoV-2, with an inhibitor compound shown at the active site.

FIG. 2: Binding site of homology model of SARS-CoV-2 3CL with a core-docked ligand (Compound B, N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide).

FIG. 3: Fit between predicted ΔG COVID-19 compared to FRET-based IC50 values against SARS.

FIG. 4: Representative thermal shift binding data of N-((1S)-1-{[((1S)-3-hydroxy-2-oxo-1-{[(3S)-2-oxopyrrolidin-3-yl]methyl} propyl)amino] carbonyl}-3-methylbutyl)-4-methoxy-1H-indole-2-carboxamide with SARS-CoV-2 3CLpro.

FIG. 5: 3-dimensional depiction of antiviral activity of N-((1S)-1-{[((1S)-3-hydroxy-2-oxo-1-{[(3S)-2-oxopyrrolidin-3-yl]methyl} propyl)amino] carbonyl}-3-methylbutyl)-4-methoxy-1H-indole-2-carboxamide in combination with remdesivir against SARS-CoV-2.

FIG. 5A: Activity (%) as a function of concentration of N-((1S)-1-{[((1S)-3-hydroxy-2-oxo-1-{[(3S)-2-oxopyrrolidin-3-yl]methyl} propyl)amino] carbonyl}-3-methylbutyl)-4-methoxy-1H-indole-2-carboxamide in presence of remdesivir at 0 nm, 48 nm, 95 nm and 190 nm.

FIG. 6: PXRD pattern of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide, hydrate, Form 3.

FIG. 7: PXRD pattern of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide, Form 1.

FIG. 8: PXRD pattern of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide, Form 2.

FIG. 9: 13C solid state NMR spectrum of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide, Form 2.

FIG. 10: Visual observations of precipitation were recorded for formulations of varied PF-00835231 concentration in solutions contain varied volume fractions of PEG400 relative to ethanol.

FIG. 11: Total co-solvent percent (% v/v) vs. cyclodextrin to PF-00835231 ratio FIG. 12: Assay values of PF-00835231 formulations containing ethanol, PEG400 and SBE-β-cyclodextrin and 2, 4, 6 and 8 mg/mL PF-00835231 over 7 days.

FIG. 13: Stability of 80 mg/mL SBE-β-cyclodextrin, 4.5% v/v total co-solvent (1.1% v/v ethanol, 3.4% v/v PEG400), and 6 mg/mL PF-00835231 solution at −20° C. (top), 4° C. (middle) and 25° C. (bottom).

FIG. 14: Chemical stability of PF-00835231 in solutions with CD (dashed) and without CD (solid) at pH 4 (black, open circles) and pH 5 (gray, closed circles) at 40° C. (top) and 22° C. (bottom).

### DETAILED DESCRIPTION OF THE INVENTION

For the purposes of the present invention, as described and claimed herein, the following terms are defined as follows:

As used herein, the terms “comprising” and “including” are used in their open, non-limiting sense. The term “treating”, as used herein, unless otherwise indicated, means reversing, alleviating, inhibiting the progress of, or preventing the disorder of condition to which such term applies, or one or more symptoms of such disorder or condition. In the methods of treating COVID-19 it is to be understood that COVID-19 is the disease caused in patients by infection with the SARS-CoV-2 virus. The SARS-CoV-2 virus is to be understood to encompass the initially discovered strain of the virus as well as mutant strains which emerge, such as but not limited to, strains such as B.1.1.7 (UK variant), B.1.351 (South African variant) and P.1 (Brazilian variant). The term “treatment”, as used herein, unless otherwise indicated, refers to the act of treating as “treating” is defined immediately above.

The phrase “pharmaceutically acceptable salts(s)”, as used herein, unless otherwise indicated, includes salts of acidic or basic groups which may be present in the compounds described herein. The compounds used in the methods of the invention that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isethionate, lactate, lactobionate, laurate, malate, maleate, mandelate, mesylate, methylsulfate, mucate, napsylate, nitrate, oleate, oxalate, pamoate (embonate), palmitate, pantothenate, phosphate/diphosphate, polygalacturonate, salicylate, stearate, subacetate, succinate, tannate, tartrate, teoclate, tosylate, triethiodode, and valerate salts. In addition to existing as pharmaceutically acceptable salts, it is to be understood that compounds used in the invention may exist as co-crystals.

With respect to the compounds used in the methods of the invention, if the compounds also exist as tautomeric forms then this invention relates to the use of all such tautomers and mixtures thereof.

The subject invention also includes methods of treatment of COVID-19 and methods of inhibiting SARS-CoV-2 with isotopically-labelled compounds, which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or isotopes of other atoms are with the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds used in the methods of this invention and prodrugs thereof can generally be prepared by carrying out the procedures for preparing the compounds disclosed in the art by substituting a readily available isotopically labelled reagent for a non-isotopically labelled reagent.

This invention also encompasses methods using pharmaceutical compositions and methods of treating COVID-19 infections through administering prodrugs of compounds of the invention. Compounds having free amido or hydroxy groups can be converted into prodrugs. Prodrugs include compounds wherein an amino acid residue, or a polypeptide chain of two or more (e.g., two, three or four) amino acid residues is covalently joined through an ester bond to a hydroxy of compounds used in the methods of this invention. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs are also encompassed. For instance, free hydroxy groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem., 1996, 29, 10. Free amines can also be derivatized as amides, sulfonamides or phosphonamides. All of these prodrug moieties may incorporate groups including but not limited to ether, amine and carboxylic acid functionalities.

The compounds of the invention can also be used in combination with other drugs. For example, dosing a SARS-CoV-2 coronavirus infected patient (i.e. a patient with COVID-19) with the SARS-CoV-2 coronavirus 3CL protease inhibitor of the invention and an interferon, such as interferon alpha, or a pegylated interferon, such as PEG-Intron or Pegasus, may provide a greater clinical benefit than dosing either the interferon, pegylated interferon or the SARS-CoV-2 coronavirus inhibitor alone. Examples of greater clinical benefits could include a larger reduction in COVID-19 symptoms, a faster time to alleviation of symptoms, reduced lung pathology, a larger reduction in the amount of SARS-CoV-2 coronavirus in the patient (viral load), and decreased mortality.

The SARS-CoV-2 coronavirus infects cells which express p-glycoprotein. Some of the SARS-CoV-2 coronavirus 3CL protease inhibitors of the invention are p-glycoprotein substrates. Compounds which inhibit the SARS-CoV-2 coronavirus which are also p-glycoprotein substrates may be dosed with p-glycoprotein inhibitor. Examples of p-glycoprotein inhibitors are verapamil, vinblastine, ketoconazole, nelfinavir, ritonavir or cyclosporine. The p-glycoprotein inhibitors act by inhibiting the efflux of the SARS-CoV-2 coronavirus inhibitors of the invention out of the cell. The inhibition of the p-glycoprotein based efflux will prevent reduction of intracellular concentrations of the SARS-CoV-2 coronavirus inhibitor due to p-glycoprotein efflux. Inhibition of the p-glycoprotein efflux will result in larger intracellular concentrations of the SARS-CoV-2 coronavirus inhibitors. Dosing a SARS-CoV-2 coronavirus infected patient with the SARS-CoV-2 coronavirus 3CL protease inhibitors of the invention and a p-glycoprotein inhibitor may lower the amount of SARS-CoV-2 coronavirus 3CL protease inhibitor required to achieve an efficacious dose by increasing the intracellular concentration of the SARS-CoV-2 coronavirus 3CL protease inhibitor.

Among the agents that may be used to increase the exposure of a mammal to a compound of the present invention are those that can as inhibitors of at least one isoform of the cytochrome P450 (CYP450) enzymes. The isoforms of CYP450 that may be beneficially inhibited included, but are not limited to CYP1A2, CYP2D6, CYP2C9, CYP2C19 and CYP3A4. The compounds used in the methods of the invention include compounds that may be CYP3A4 substrates and are metabolized by CYP3A4. Dosing a SARS-CoV-2 coronavirus infected patient with a SARS-CoV-2 coronavirus inhibitor which is a CYP3A4 substrate, such as SARS-CoV-2 coronavirus 3CL protease inhibitor, and a CYP3A4 inhibitor, such as ritonavir, nelfinavir or delavirdine, will reduce the metabolism of the SARS-Cov-2 coronavirus inhibitor by CYP3A4. This will result in reduced clearance of the SARS-CoV-2 coronavirus inhibitor and increased SARS-Cov-2 coronavirus inhibitor plasma concentrations. The reduced clearance and higher plasma concentrations may result in a lower efficacious dose of the SARS-CoV-2 coronavirus inhibitor.

Additional therapeutic agents that can be used in combination with the SARS-CoV-2 inhibitors in the methods of the present invention include the following:

PLpro inhibitors: Ribavirin, Valganciclovir, β-Thymidine, Aspartame, Oxprenolol, Doxycycline, Acetophenazine, lopromide, Riboflavin, Reproterol, 2,2′-Cyclocytidine, Chloramphenicol, Chlorphenesin carbamate, Levodropropizine, Cefamandole, Floxuridine, Tigecycline, Pemetrexed, L(+)-Ascorbic acid, Glutathione, Hesperetin, Ademetionine, Masoprocol, Isotretinoin, Dantrolene, Sulfasalazine Anti-bacterial, Silybin, Nicardipine, Sildenafil, Platycodin, Chrysin, Neohesperidin, Baicalin, Sugetriol-3,9-diacetate, (−)-Epigallocatechin gallate, Phaitanthrin D, 2-(3,4-Dihydroxyphenyl)-2-[[2-(3,4-dihydroxyphenyl)-3,4-dihydro-5,7-dihydroxy-2H-1-benzopyran-3-yl]oxy]-3,4-dihydro-2H-1-benzopyran-3,4,5,7-tetrol, 2,2-Di(3-indolyl)-3-indolone, (S)-(1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl-2-amino-3-phenylpropanoate, Piceatannol, Rosmarinic acid, and Magnolol.

3CLpro inhibitors: Lymecycline, Chlorhexidine, Alfuzosin, Cilastatin, Famotidine, Almitrine, Progabide, Nepafenac, Carvedilol, Amprenavir, Tigecycline, Montelukast, Carminic acid, Mimosine, Flavin, Lutein, Cefpiramide, Phenethicillin, Candoxatril, Nicardipine, Estradiol valerate, Pioglitazone, Conivaptan, Telmisartan, Doxycycline, Oxytetracycline, (1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl5-((R)-1,2-dithiolan-3-yl) pentanoate, Betulonal, Chrysin-7-O-β-glucuronide, Andrographiside, (1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl 2-nitrobenzoate, 2β-Hydroxy-3,4-seco-friedelolactone-27-oic acid (S)-(1 S,2R,4aS,5R,8aS)-1-Formamido-1,4a-dimethyl-6-methylene-5-((E)-2-(2-oxo-2,5-dihydrofuran-3-yl)ethenyl) decahydronaphthalen-2-yl-2-amino-3-phenylpropanoate, Isodecortinol, Cerevisterol, Hesperidin, Neohesperidin, Andrograpanin, 2-((1R,5R,6R,8aS)-6-Hydroxy-5-(hydroxymethyl)-5,8a-dimethyl-2-methylenedecahydronaphthalen-1-yl)ethyl benzoate, Cosmosiin, Cleistocaltone A, 2,2-Di(3-indolyl)-3-indolone, Biorobin, Gnidicin, Phyllaemblinol, Theaflavin 3,3′-di-O-gallate, Rosmarinic acid, Kouitchenside I, Oleanolic acid, Stigmast-5-en-3-ol, Deacetylcentapicrin, and Berchemol.

RdRp inhibitors: Valganciclovir, Chlorhexidine, Ceftibuten, Fenoterol, Fludarabine, Itraconazole, Cefuroxime, Atovaquone, Chenodeoxycholic acid, Cromolyn, Pancuronium bromide, Cortisone, Tibolone, Novobiocin, Silybin, Idarubicin Bromocriptine, Diphenoxylate, Benzylpenicilloyl G, Dabigatran etexilate, Betulonal, Gnidicin, 2β,30β-Dihydroxy-3,4-seco-friedelolactone-27-Iactone, 14-Deoxy-11,12-didehydroandrographolide, Gniditrin, Theaflavin 3,3′-di-O-gallate, (R)-((1R,5aS,6R,9aS)-1,5a-Dimethyl-7-methylene-3-oxo-6-((E)-2-(2-oxo-2,5-dihydrofuran-3-yl)ethenyl)decahydro-1H-benzo[c]azepin-1-yl)methyl2-amino-3-phenylpropanoate, 2β-Hydroxy-3,4-seco-friedelolactone-27-oic acid, 2-(3,4-Dihydroxyphenyl)-2-[[2-(3,4-dihydroxyphenyl)-3,4-dihydro-5,7-dihydroxy-2H-1-benzopyran-3-yl]oxy]-3,4-dihydro-2H-1-benzopyran-3,4,5,7-tetrol, Phyllaemblicin B, 14-hydroxycyperotundone, Andrographiside, 2-((1R,5R,6R,8aS)-6-Hydroxy-5-(hydroxymethyl)-5,8a-dimethyl-2-methylenedecahydro naphthalen-1-yl)ethyl benzoate, Andrographolide, Sugetriol-3,9-diacetate, Baicalin, (1S,2R,4aS,5R,8aS)-1-Formamido-1,4a-dimethyl-6-methylene-5-((E)-2-(2-oxo-2,5-dihydrofuran-3-yl)ethenyl)decahydronaphthalen-2-yl 5-((R)-1,2-dithiolan-3-yl)pentanoate, 1,7-Dihydroxy-3-methoxyxanthone, 1,2,6-Trimethoxy-8-[(6-O-β-D-xylopyranosyl-β-D-glucopyranosyl)oxy]-9H-xanthen-9-one, and 1,8-Dihydroxy-6-methoxy-2-[(6-O-β-D-xylopyranosyl-β-D-glucopyranosyl)oxy]-9H-xanthen-9-one, 8-(β-D-Glucopyranosyloxy)-1,3,5-trihydroxy-9H-xanthen-9-one,

Additional therapeutic agents that can be used in the methods of the invention include Diosmin, Hesperidin, MK-3207, Venetoclax, Dihydroergocristine, Bolazine, R428, Ditercalinium, Etoposide, Teniposide, UK-432097, Irinotecan, Lumacaftor, Velpatasvir, Eluxadoline, Ledipasvir, Lopinavir/Ritonavir+Ribavirin, Alferon, and prednisone.

Other additional agents that can be used in the methods of the present invention include α-ketoamides compounds designated as 11r, 13a and 13b, shown below, as described in Zhang, L.; Lin, D.; Sun, X.; Rox, K.; Hilgenfeld, R.; X-ray Structure of Main Protease of the Novel Coronavirus SARS-CoV-2 Enables Design of α-Ketoamide Inhibitors; bioRxiv preprint doi: https://doi.org/10.1101/2020.02.17.952879

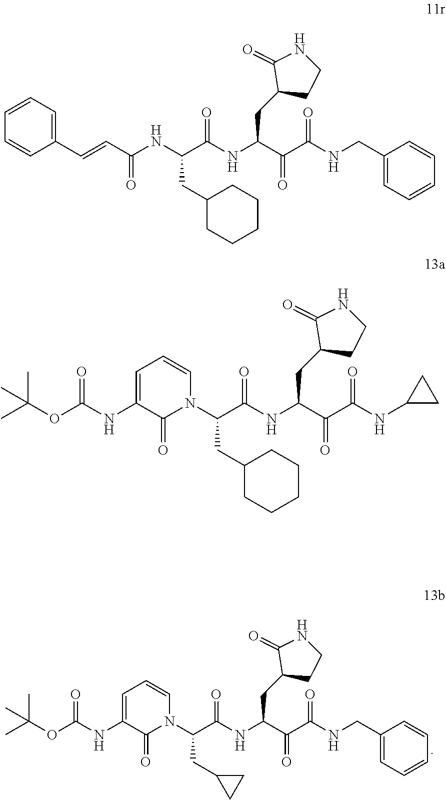 [SMILES: O=C(/C=C/c1ccccc1)N[C@@H](CC1CCCCC1)C(=O)N[C@@H](C[C@@H]1CCNC1=O)C(=O)C(=O)NCc1ccccc1 ; CC(C)(C)OC(=O)Nc1cccn([C@@H](CC2CCCCC2)C(=O)N[C@@H](C[C@@H]2CCNC2=O)C(=O)C(=O)NC2CC2)c1=O ; CC(C)(C)OC(=O)Nc1cccn([C@@H](CC2CC2)C(=O)N[C@@H](C[C@@H]2CCNC2=O)C(=O)C(=O)NCc2ccccc2)c1=O]

Other additional agents that can be used in the methods of the present invention include chloroquine, hydroxychloroquine, azithromycin and remdesivir. Examples of greater clinical benefits could include a larger reduction in COVID-19 symptoms, a faster time to alleviation of symptoms, reduced lung pathology, a larger reduction in the amount of SARS-Cov-2 coronavirus in the patient (viral load), and decreased mortality.

Another embodiment of the present invention is a method of treating COVID-19 in a patient wherein an additional agent is administered and the additional agent is selected from antivirals such as remdesivir, galidesivir, favilavir/avifavir, mulnupiravir (MK-4482/EIDD 2801), AT-527, AT-301, BLD-2660, favipiravir, camostat, SLV213 emtrictabine/tenofivir, clevudine, dalcetrapib, boceprevir and ABX464, glucocorticoids such as dexamethasone and hydrocortisone, convalescent plasma, a recombinant human plasma such as gelsolin (Rhu-p65N), monoclonal antibodies such as regdanvimab (Regkirova), ravulizumab (Ultomiris), VIR-7831/VIR-7832, BRII-196/BRII-198, COVI-AMG/COVI DROPS (STI-2020), bamlanivimab (LY-CoV555), mavrilimab, leronlimab (PRO140), AZD7442, lenzilumab, infliximab, adalimumab, JS 016, STI-1499 (COVIGUARD), lanadelumab (Takhzyro), canakinumab (Ilaris), gimsilumab and otilimab, antibody cocktails such as casirivimab/imdevimab (REGN-Cov2), recombinant fusion protein such as MK-7110 (CD24Fc/SACCOVID), anticoagulants such as heparin and apixaban, IL-6 receptor agonists such as tocilizumab (Actemra) and sarilumab (Kevzara), PlKfyve inhibitors such as apilimod dimesylate, RIPK1 inhibitors such as DNL758, VIP receptor agonists such as PB1046, SGLT2 inhibitors such as dapaglifozin, TYK inhibitors such as abivertinib, kinase inhibitors such as ATR-002, bemcentinib, acalabrutinib and losmapimod, H2 blockers such as famotidine, anthelmintics such as niclosamide, furin inhibitors such as diminazene.

The term “SARS-CoV-2 inhibiting agent” means any SARS-CoV-2 related coronavirus 3CL protease inhibitor compound described herein or a pharmaceutically acceptable salt, hydrate, prodrug, active metabolite or solvate thereof or a compound which inhibits replication of SARS-CoV-2 in any manner.

The term “interfering with or preventing” SARS-CoV-2-related coronavirus (“SARS-CoV-2”) viral replication in a cell means to reduce SARS-CoV-2 replication or production of SARS-CoV-2 components necessary for progeny virus in a cell as compared to a cell not being transiently or stably transduced with the ribozyme or a vector encoding the ribozyme. Simple and convenient assays to determine if SARS-CoV-2 viral replication has been reduced include an ELISA assay for the presence, absence, or reduced presence of anti-SARS-CoV-2 antibodies in the blood of the subject (Nasoff, et al., PNAS 88:5462-5466, 1991), RT-PCR (Yu, et al., in Viral Hepatitis and Liver Disease 574-577, Nishioka, Suzuki and Mishiro (Eds.); Springer-Verlag, Tokyo, 1994). Such methods are well known to those of ordinary skill in the art. Alternatively, total RNA from transduced and infected “control” cells can be isolated and subjected to analysis by dot blot or northern blot and probed with SARS-CoV-2 specific DNA to determine if SARS-CoV-2 replication is reduced. Alternatively, reduction of SARS-CoV-2 protein expression can also be used as an indicator of inhibition of SARS-CoV-2 replication. A greater than fifty percent reduction in SARS-CoV-2 replication as compared to control cells typically quantitates a prevention of SARS-CoV-2 replication.

If an SARS-CoV-2 inhibitor compound used in the method of the invention is a base, a desired salt may be prepared by any suitable method known to the art, including treatment of the free base with an inorganic acid (such as hydrochloric acid, hydrobromic acid, sulfuric acid, nitric acid, phosphoric acid, and the like), or with an organic acid (such as acetic acid, maleic acid, succinic acid, mandelic acid, fumaric acid, malonic acid, pyruvic acid, oxalic acid, glycolic acid, salicylic acid, pyranosidyl acid (such as glucuronic acid or galacturonic acid), alpha-hydroxy acid (such as citric acid or tartaric acid), amino acid (such as aspartic acid or glutamic acid), aromatic acid (such as benzoic acid or cinnamic acid), sulfonic acid (such as p-toluenesulfonic acid or ethanesulfonic acid), and the like.

If a SARS-CoV-2 inhibitor compound used in the method of the invention is an acid, a desired salt may be prepared by any suitable method known to the art, including treatment of the free acid with an inorganic or organic base (such as an amine (primary, secondary, or tertiary)), an alkali metal hydroxide, or alkaline earth metal hydroxide. Illustrative examples of suitable salts include organic salts derived from amino acids (such as glycine and arginine), ammonia, primary amines, secondary amines, tertiary amines, and cyclic amines (such as piperidine, morpholine, and piperazine), as well as inorganic salts derived from sodium, calcium, potassium, magnesium, manganese, iron, copper, zinc, aluminum and lithium.

In the case of SARS-CoV-2 inhibitor compounds, prodrugs, salts, or solvates that are solids, it is understood by those skilled in the art that the hydroxamate compound, prodrugs, salts, and solvates used in the method of the invention, may exist in different polymorph or crystal forms, all of which are intended to be within the scope of the present invention and specified formulas. In addition, the hydroxamate compound, salts, prodrugs and solvates used in the method of the invention may exist as tautomers, all of which are intended to be within the broad scope of the present invention.

In some cases, the SARS-CoV-2 inhibitor compounds, salts, prodrugs and solvates used in the method of the invention may have chiral centers. When chiral centers are present, the hydroxamate compound, salts, prodrugs and solvates may exist as single stereoisomers, racemates, and/or mixtures of enantiomers and/or disastereomers. All such single stereoisomers, racemates, and mixtures thereof are intended to be within the broad scope of the present invention.

As generally understood by those skilled in the art, an optically pure compound is one that is enantiomerically pure. As used herein, the term “optically pure” is intended to mean a compound comprising at least a sufficient activity. Preferably, an optically pure amount of a single enantiomer to yield a compound having the desired pharmacological pure compound of the invention comprised at least 90% of a single isomer (80% enantiomeric excess), more preferably at least 95% (90% e.e.), even more preferably at least 97.5% (95%) e.e.), and most preferably at least 99% (98% e.e.).

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. In a preferred embodiment of the present invention, “treating” or “treatment” means at least the mitigation of a disease condition in a human, that is alleviated by the inhibition of the activity of the SARS-CoV-2 3C-like protease which is the main protease of SARS-CoV-2, the causative agent for COVID-19. For patients suffering from COVID-19 fever, fatigue, and dry cough are the main manifestations of the disease, while nasal congestion, runny nose, and other symptoms of the upper respiratory tract are rare. Beijing Centers for Diseases Control and Prevention indicated that the typical case of COVID-19 has a progressive aggravation process. COVID-19 can be classified into light, normal, severe, and critical types based on the severity of the disease National Health Commission of the People's Republic of China. Diagnosis and Treatment of Pneumonia Caused by 2019-nCoV (Trial Version 4). Available online: http://www.nhc.gov.cn/jkj/s3577/202002/573340613ab243b3a7f61df260551dd4/files/c791e5a7ea5149f680fdcb34dac0f54e.pdf (accessed on 6 Feb. 2020): (1) Mild cases—the clinical symptoms were mild, and no pneumonia was found on the chest computed tomography (CT); (2) normal cases—fever, respiratory symptoms, and patients found to have imaging manifestations of pneumonia; (3) severe cases—one of the following three conditions: Respiratory distress, respiratory rate≥30 times/min (in resting state, refers to oxygen saturation s 93%), partial arterial oxygen pressure (PaO2)/oxygen absorption concentration (FiO2) ≥300 mmHg (1 mmHg=0.133 kPa); (4) critical cases—one of the following three conditions: Respiratory failure and the need for mechanical ventilation, shock, or the associated failure of other organs requiring the intensive care unit. The current clinical data shows that the majority of the deaths occurred in the older patients. However, severe cases have been documented in young adults who have unique factors, particularly those with chronic diseases, such as diabetes or hepatitis B. Those with a long-term use of hormones or immunosuppressants, and decreased immune function, are likely to get severely infected.

Methods of treatment for mitigation of a disease condition such as COVID-19 include the use of one or more of the compounds in the invention in any conventionally acceptable manner. According to certain preferred embodiments of the invention, the compound or compounds used in the methods of the present invention are administered to a mammal, such as a human, in need thereof. Preferably, the mammal in need thereof is infected with a coronavirus such as the causative agent of COVID-19, namely SARS-CoV-2.

The present invention also includes prophylactic methods, comprising administering an effective amount of a SARS-CoV-2 inhibitor of the invention, or a pharmaceutically acceptable salt, prodrug, pharmaceutically active metabolite, or solvate thereof to a mammal, such as a human at risk for infection by SARS-CoV-2.

According to certain preferred embodiments, an effective amount of one or more compounds of the invention, or a pharmaceutically acceptable salt, prodrug, pharmaceutically active metabolite, or solvate thereof is administered to a human at risk for infection by SARS-CoV-2, the causative agent for COVID-19. The prophylactic methods of the invention include the use of one or more of the compounds in the invention in any conventionally acceptable manner.

The following are examples of specific embodiments of the invention:

Certain of the compounds used in the methods of the invention are known and can be made by methods known in the art.

Recent evidence indicates that a new coronavirus SARS-Cov-2 is the causative agent of COVID-19. The nucleotide sequence of the SARS-CoV-2 coronavirus as well as the recently determined L- and S-subtypes have recently been determined and made publicly available.

The activity of the inhibitor compounds as inhibitors of SARS-CoV-2 viral activity may be measured by any of the suitable methods available in the art, including in vivo and in vitro assays. The activity of the compounds of the present invention as inhibitors of coronavirus 3C-like protease activity (such as the 3C-like protease of the SARS-CoV-2 coronavirus) may be measured by any of the suitable methods known to those skilled in the art, including in vivo and in vitro assays. Examples of suitable assays for activity measurements include the antiviral cell culture assays described herein as well as the antiprotease assays described herein, such as the assays described in the Example section.

Administration of the SARS-CoV-2 inhibitor compounds and their pharmaceutically acceptable prodrugs, salts, active metabolites, and solvates may be performed according to any of the accepted modes of administration available to those skilled in the art. Illustrative examples of suitable modes of administration include oral, nasal, pulmonary, parenteral, topical, intravenous, injected, transdermal, and rectal. Oral, intravenous, and nasal deliveries are preferred.

A SARS-CoV-2-inhibiting agent may be administered as a pharmaceutical composition in any suitable pharmaceutical form. Suitable pharmaceutical forms include solid, semisolid, liquid, or lyophilized formulations, such as tablets, powders, capsules, suppositories, suspensions, liposomes, and aerosols. The SARS-CoV-2-inhibiting agent may be prepared as a solution using any of a variety of methodologies. For example, SARS-CoV-2-inhibiting agent can be dissolved with acid (e.g., 1 M HCl) and diluted with a sufficient volume of a solution of 5% dextrose in water (D5W) to yield the desired final concentration of SARS-Cov-2-inhibiting agent (e.g., about 15 mM). Alternatively, a solution of D5W containing about 15 mM HCl can be used to provide a solution of the SARS-CoV-2-inhibiting agent at the appropriate concentration. Further, the SARS-Cov-2-inhibiting agent can be prepared as a suspension using, for example, a 1% solution of carboxymethylcellulose (CMC).

Acceptable methods of preparing suitable pharmaceutical forms of the pharmaceutical compositions are known or may be routinely determined by those skilled in the art. For example, pharmaceutical preparations may be prepared following conventional techniques of the pharmaceutical chemist involving steps such as mixing, granulating, and compressing when necessary for tablet forms, or mixing, filling and dissolving the ingredients as appropriate, to give the desired products for intravenous, oral, parenteral, topical, intravaginal, intranasal, intrabronchial, intraocular, intraaural, and/or rectal administration.

Pharmaceutical compositions of the invention may also include suitable excipients, diluents, vehicles, and carriers, as well as other pharmaceutically active agents, depending upon the intended use. Solid or liquid pharmaceutically acceptable carriers, diluents, vehicles, or excipients may be employed in the pharmaceutical compositions. Illustrative solid carriers include starch, lactose, calcium sulfate dihydrate, terra alba, sucrose, talc, gelatin, pectin, acacia, magnesium stearate, and stearic acid. Illustrative liquid carriers include syrup, peanut oil, olive oil, saline solution, and water. The carrier or diluent may include a suitable prolonged-release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., solution), or a nonaqueous or aqueous liquid suspension.

A dose of the pharmaceutical composition may contain at least a therapeutically effective amount of a SARS-CoV-2-inhibiting agent and preferably is made up of one or more pharmaceutical dosage units. The selected dose may be administered to a mammal, for example, a human patient, in need of treatment mediated by inhibition of SARS-related coronavirus activity, by any known or suitable method of administering the dose, including topically, for example, as an ointment or cream; orally; rectally, for example, as a suppository; parenterally by injection; intravenously; or continuously by intravaginal, intranasal, intrabronchial, intraaural, or intraocular infusion.

The phrases “therapeutically effective amount” and “effective amount” are intended to mean the amount of an inventive agent that, when administered to a mammal in need of treatment, is sufficient to effect treatment for injury or disease conditions alleviated by the inhibition of SARS-CoV-2 viral replication. The amount of a given SARS-CoV-2-inhibiting agent used in the method of the invention that will be therapeutically effective will vary depending upon factors such as the particular SARS-CoV-2-inhibiting agent, the disease condition and the severity thereof, the identity and characteristics of the mammal in need thereof, which amount may be routinely determined by those skilled in the art.

It will be appreciated that the actual dosages of the SARS-CoV-2-inhibiting agents used in the pharmaceutical compositions of this invention will be selected according to the properties of the particular agent being used, the particular composition formulated, the mode of administration and the particular site, and the host and condition being treated. Optimal dosages for a given set of conditions can be ascertained by those skilled in the art using conventional dosage-determination tests. For oral administration, e.g., a dose that may be employed is from about 0.01 to about 1000 mg/kg body weight, preferably from about 0.1 to about 500 mg/kg body weight, and even more preferably from about 1 to about 500 mg/kg body weight, with courses of treatment repeated at appropriate intervals. For intravenous dosing a dose of up to 5 grams per day may be employed. Intravenous administration can occur for intermittent periods during a day or continuously over a 24-hour period.

The terms “cytochrome P450-inhibiting amount” and “cytochrome P450 enzyme activity-inhibiting amount”, as used herein, refer to an amount of a compound required to decrease the activity of cytochrome P450 enzymes or a particular cytochrome P450 enzyme isoform in the presence of such compound. Whether a particular compound of decreases cytochrome P450 enzyme activity, and the amount of such a compound required to do so, can be determined by methods know to those of ordinary skill in the art and the methods described herein.

Protein functions required for coronavirus replication and transcription are encoded by the so-called “replicase” gene. Two overlapping polyproteins are translated from this gene and extensively processed by viral proteases. The C-proximal region is processed at eleven conserved interdomain junctions by the coronavirus main or “3C-like protease. The name “3C-like” protease derives from certain similarities between the coronavirus enzyme and the well-known picornavirus 3C proteases. These include substrate preferences, use of cysteine as an active site nucleophile in catalysis, and similarities in their putative overall polypeptide folds. A comparison of the amino acid sequence of the SARS-Cov-2-associated coronavirus 3C-like protease to that of other known coronaviruses such as SARS-CoV shows the amino acid sequences have approximately 96% shared homology.

Amino acids of the substrate in the protease cleavage site are numbered from the N to the C terminus as follows: -P3-P2-P1-P1′-P2′-P3′, with cleavage occurring between the P1 and P1′ residues (Schechter & Berger, 1967). Substrate specificity is largely determined by the P2, P1 and P1′ positions. Coronavirus main protease cleavage site specificities are highly conserved with a requirement for glutamine at P1 and a small amino acid at P1′ (Journal of General Virology, 83, pp. 595-599 (2002)).

### EXAMPLES

The compound (3S)-3-({4-methyl-N-[(2R)-tetrahydrofuran-2-ylcarbonyl]-L-leucyl}amino)-2-oxo-4-[(3S)-2-oxopyrrolidin-3-yl]butyl 2,6-dichlorobenzoate; can be prepared as set forth in Example 39 of WO2005/113580 and as reproduced below. The compound (3S)-3-({N-[(4-methoxy-1H-indol-2-yl)carbonyl]-L-leucyl}amino)-2-oxo-4-[(3S)-2-oxopyrrolidin-3-yl]butyl cyclopropanecarboxylate; can be prepared as set forth in Example 37 of WO2005/113580 and as reproduced below. The compound N-((1S)-1-{[((1S)-3-hydroxy-2-oxo-1-{[(3S)-2-oxopyrrolidin-3-yl]methyl}propyl)amino]carbonyl}-3,3-dimethylbutyl)-1H-indole-2-carboxamide; can be prepared as set forth in Example 16 of WO2005/113580 and as reproduced below. The compound N-((1S)-1-{[((1S)-3-hydroxy-2-oxo-1-{[(3S)-2-oxopyrrolidin-3-yl]methyl}propyl)amino]carbonyl}pentyl)-4-methoxy-1H-indole-2-carboxamide; can be prepared as set forth in Example 8 WO2005/113580 and as reproduced below. The compound N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide; can be prepared as set forth in Example 2 of WO2005/113580 and as reproduced below. These compounds are referred to as Reference Examples where reproduced below.

In the examples described below, unless otherwise indicated, all temperatures are set forth in degrees Celsius and all parts and percentages are by weight. Reagents may be purchased from commercial suppliers, such as Sigma-Aldrich Chemical Company, or Lancaster Synthesis Ltd. and may be used without further purification unless otherwise indicated. Tetrahydrofuran (THF) and N, N-dimethylformamide (DMF) may be purchased from Aldrich in Sure Seal bottles and used as received. All solvents may be purified using standard methods known to those skilled in the art, unless otherwise indicated.

The structures of the compounds of the following examples are confirmed by one or more of the following: proton magnetic resonance spectroscopy, elemental microanalysis and melting point. Proton magnetic resonance (¹H NMR) spectra are determined using a Bruker spectrometer operating at a field strength of 300 to 400 megahertz (MHz). Chemical shifts are reported in parts per million (ppm, δ) downfield from an internal tetramethylsilane standard. Alternatively, ¹H NMR spectra were referenced to residual protic solvent signals as follows: CHCl₃=7.26 ppm, DMSO=2.49 ppm, C₆HD₅=7.15 ppm. Peak multiplicities are designated as follows: s, singlet; d, doublet; dd, doublet of doublets; t, triplet; q, quartet; br, broad resonance; m, multiplet. Coupling constants are given in Hertz. Elemental microanalyses are performed by Atlantic Microlab Inc., Norcross, Ga. and gave results for the elements stated with ±0.4% of the theoretical values. Flash column chromatography is performed using Silica gel 60 (Merck Art 9385) or various MPLC systems. Analytical thin layer chromatography (TLC) was performed using precoated sheets of Silica 60 F254 (Merck Art 5719). All reactions are performed in septum-sealed flasks under a slight positive pressure of argon or dry nitrogen unless otherwise noted.

Other preferred compounds used in the methods of the invention may be prepared in manners analogous to those specifically described below.

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise notes, exist as a racemic mixture. Those molecules with two or more chiral centers, unless otherwise noted, exits as a racemic mixture of diastereomers. Single enantiomers/diastereomers may be obtained by methods known to those skilled in the art.

Where HPLC chromatography is referred to in the preparations and examples below, the general conditions used, unless otherwise indicated, are as follows. The column used is a ZORBAXμ RXC18 column (manufactured by Hewlett Packard) of 150 mm distance and 4.6 mm interior diameter. The samples are run on a Hewlett Packard-1100 systemA gradient solvent method is used running 100 percent ammonium acetate/acetic acid buffer (0.2 M) to 100 percent acetonitrile over 10 minutes. The system then proceeds on a wash cycle with 100 percent acetonitrile for 1.5 minutes and then 100 percent buffer solution for 3 minutes. The flow rate over this period is a constant 3 mL/minute.

In the examples and specification “Et” means ethyl, “Ac” means acetyl, “Me” means methyl, “ETOAC” or “ETOAc” means ethyl acetate, “THF” means tetrahydrofuran, and “Bu” means butyl Et₂O refers to diethyl ether, DMF refers to N,N-dimethylformamide. DMSO refers to dimethylsulfoxide. MTBE refers to tert-butyl methyl ether. Other abbreviations include: CH₃OH (methanol), EtOH (ethanol), EtOAc (ethyl acetate), DEM (ethylene glycol dimethyl ether) DCM refer to dichloromethane, 1,2 DCE refers to 1,2 dichloroethane, Ph (phenyl), Tr (triphenylmethyl), Cbz (benzyloxycarbonyl), Boc (tert-butoxycarbonyl), TFA (trifluoroacetic acid), DIEA (N,N-diisopropylethylamine), TMEDA (N,N,N′,N′-tetramethylethylenediamine), AcOH (acetic acid), Ac₂O (acetic anhydride), NMM (4-methylmorpholine), HOBt (1-hydroxybenzotrizole hydrate), HATU [O-(7-azabenzotriazol-1-yl)-N,N,N′,N′-tetramethyluronium hexafluorophosphate], EDC [1-(3-dimethylaminopropyl)-3-ethylcarbarbodiimide hydrochloride], TEA triethylamine, LDA lithium diisopropyl amide, DCC (dicyclohexyl-carbodiimide), DDQ (2,3-dichloro-5,6-dicyano-1,4-benzoquinone), DMAP (4-dimethylaminopyridine), Gln (glutamine), Leu (leucine), Phe (phenylalanine), Phe (4-F) (4-fluorophenylalanine), Val (valine), amino-Ala (2,3-diaminopropionic acid), and (S)-Pyrrol-Ala[(2S,3'S)-2-amino-3-(2′-oxopyrrolidin-3′-yl)-propionic acid]. Additionally, “L” represents the configuration of naturally occurring amino acids.

The following are compounds used in the methods of the invention are Examples 2, 8, 16, 23, 37 and 39 of WO2005/113580 and are referred to as Reference Examples.

### Reference Example 2: N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide

A solution of N-((1S)-1{[((1S)-3-chloro-2-oxo-1-{[3S)-2-oxopyrrolidin-3-yl]methyl}propyl)amino]carbonyl}-3-methylbutyl)-4methoxy-1H-indole-2-carboxamide (488 mg, 0.99 mmol) and benzoylformic acid (195 mg, 1.3 mmol) in DMR (6.5 mL) was placed under an atmosphere of N₂. This clear pale yellow solution was treated with cesium fluoride (350 mg, 2.3 mmol) followed by heating to 65° C. After 4 hours the now yellow suspension was cooled to RT, diluted with ethyl acetate (60 mL), washed three times water (30 mL), once with brine (30 mL), dried over MgSO₄, filtered, and concentrated to give (3S)-3-({N-[(4-methoxy-1H-indol-2-yl)carbonyl]-L-leucyl}amino-2-oxo-4-[(3S)-2-oxopyrrolidin-3-yl]butyl oxo(phenyl)acetate as a crude yellow foam. MS (ESI+) for C₃₂H₃₆N₄O₈ m/z 605.2 (M+H)⁺. A solution of the crude (3S)-3-({N-[(4-methoxy-1H-indol-2-yl)carbonyl]-L-leucyl}amino-2-oxo-4-[(3S)-2-oxopyrrolidin-3-yl]butyl oxo(phenyl)acetate in methanol (40 mL) was placed under an atmosphere of N₂ and treated with potassium carbonate (7 mg, 0.05 mmol) with vigorous stirring. After 1 hour the volatiles were removed in vacuo (bath <30° C.) to give a crude yellow glass. This material was purified by Biotage MPLC (25M column, 6% methanol/chloroform) to afford 346 mg (73%) of the title compounds as an off-white solid. ¹H NMR (DMSO-d₆) δ 11.56 (s, 1H), 8.44 (d, J=8 Hz, 1H), 8.39 (d, J=8 Hz, 1H), 7.61 (s, 1H), 7.35 (s, 1H), 7.08 (t, J=8 Hz, 1H), 6.99 (d, J=8 Hz, 1H), 6.49 (d, J=8 Hz, 1H), 5.04 (t, J=8 Hz, 1H), 4.46 (m, 2H), 4.25 (dd, J=8, 20 Hz, 1H), 4.13 (dd, J=8, 20 Hz, 1H), 3.87 (s, 3H), 3.10 (m, 2H), 2.28 (m, 1H), 2.08 (m, 1H), 1.92 (m, 1H), 1.70-1.53 (m, 5H), 0.93 (d, J=8 Hz, 3H), 0.89 (d, J=8 Hz, 3H); MS (ESI+) for C₂₄H₃₂N₄O₆ m/z 473.2 (M+H)⁺.

The following are solid form Examples of the present invention:

### Solid Form Examples of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide Form 1 and Form 2 of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide

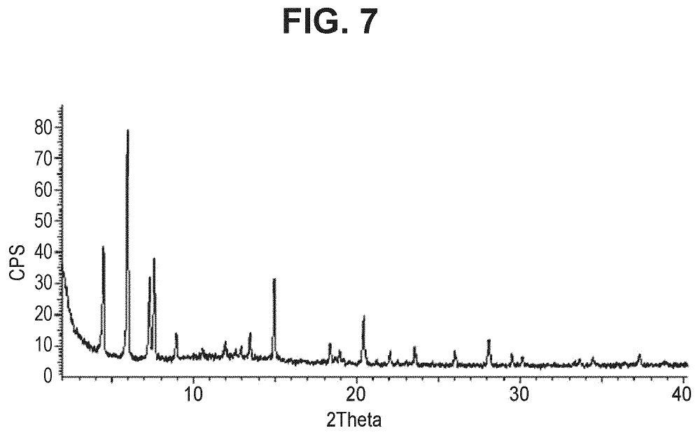

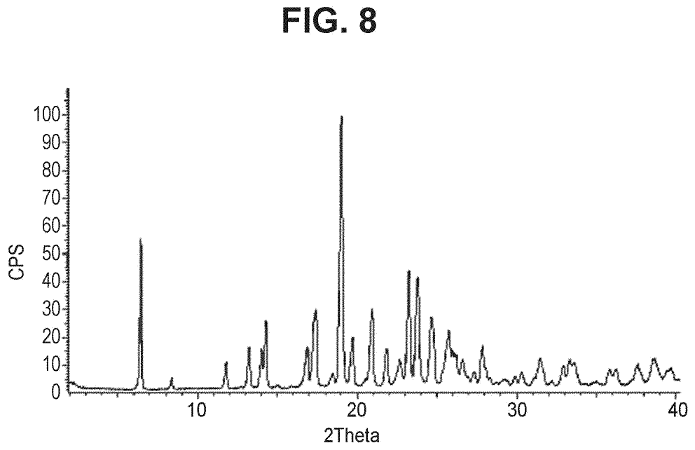

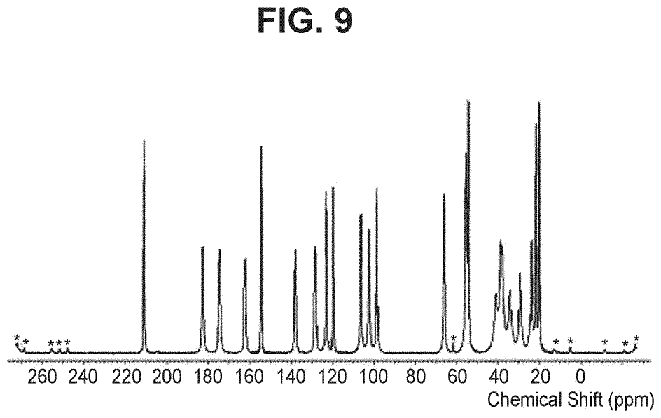

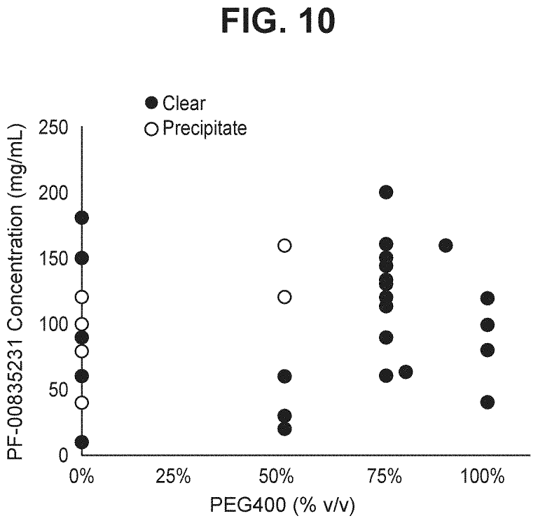

Materials preparation: Extended storage of a sample of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide resulted in a crystalline form denoted as Form 2 after testing by PXRD (shown in FIG. 8). Approximately 9 mg of this lot were suspended in 1 mL THF:toluene (3:7 vol/vol) by adding aliquots of 100 μL and vortexing the sample after each addition. Material dissolution was not observed, and the vial were placed on a roller mixer at 40° C. After 30 minutes of equilibration the solids were filtered using centrifuge filter tubes, analysed by PXRD and denoted as Form 1 (shown in FIG. 7)

### Preparation of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide, hydrate (Form 3)

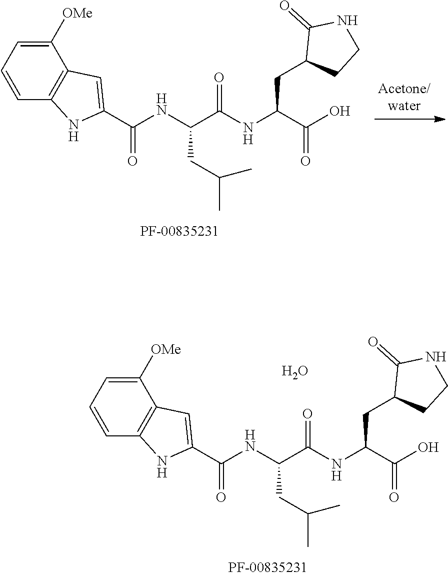 [SMILES: COc1cccc2[nH]c(C(=O)N[C@@H](CC(C)C)C(=O)N[C@@H](C[C@@H]3CCNC3=O)C(=O)O)cc12 ; COc1cccc2[nH]c(C(=O)N[C@@H](CC(C)C)C(=O)N[C@@H](C[C@@H]3CCNC3=O)C(=O)O)cc12 ; O]

Example of Form 3 Unseeded crystallization process is depicted above. A jacketed reactor at 20° C. was charged with PF-00835231 (1.0 eq, 2.0 g), Acetone (9.2 mL, 4.6 mL/g) and water (1.6 mL, 0.81 mL/g). The mixture was stirred at 20 C to afford clear solution. Additional water (4.1 mL; 2 mL/g) added slowly still resulted in a clear solution. The solvent was removed under vacuum to provide gummy solids. Acetone (9 mL; 4.5 mL/g) was added and slurry was heated to reflux. Water (20 mL; 10 mL/g) is slowly added to crystallize the product, followed by addition of more water (30 mL; 15 mL/g) over 1 h. The resulting slurry was cooled to 10° C. over 1 h and granulated for a minimum of 1 h before filtering and washing. The solids were dried at 20° C. for 1 h to provide PF-00835231 hydrate (Form 3) in 85% yield.

Example of Form 3 Seeded process: 2.8 mL of a pre-prepared water/acetone (15:85, v/v) solution was added to 1.85 g of PF-00835231 in an 8-dram vial. A stir bar was added, and the vial placed on a stirrer plate (˜500 rpm). Approximately 5 mg of Form 3 seed crystals were added and solid was observed to precipitate over a few minutes to produce a slurry. 3.8 mL water was added dropwise to the stirred slurry over about 5 minutes, then the vial was sealed and left to stir at ambient conditions for about 5 hours. The slurry was filtered, and the solid residues washed with approximately 6 mL water. Solid residue was transferred to a clean 4-dram vial and placed in a vacuum oven at 50° C. Solid product was characterisation by PXRD and confirmed consistent with Form 3. This characterization was used for creating a peak table and selection of characteristic peaks. Single crystal work has shown that the crystalline Form 3 has a 1:1 stoichiometry of water: N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide but at certain storage conditions the stoichiometry may get to 1.2:1 of water: N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide.

### Powder X-Ray Diffraction

The powder X-ray diffraction patterns for Forms 1, 2 and 3 of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide were generated using a Bruker AXS D8 Endeavor diffractometer equipped with a Cu radiation source. The tube voltage and amperage were set to 40 kV and 40 mA, respectively. The motorized divergence slits were set at constant illumination of 11 mm. Diffracted radiation was detected using a LYNXEYE XE-T energy dispersive X-ray detector, with the position sensitive detector (PSD) opening set at 4.00°. Data was collected on the theta-theta goniometer at the Cu K alpha wavelength from 2.0 to 55.0 degrees 2-theta (°2θ) using a step size of 0.019°2θ and a time per step of 0.1 seconds. Samples were prepared for analysis by placing them in a silicon low background flat holder and rotated at 15 rpm during data collection. Data were analyzed in DIFFRAC.EVA v 4.2 software. Peak lists were prepared using reflections with a relative intensity ≥5% of the most intense band in each respective diffraction pattern. A typical error of ±0.2°2θ in peak positions (USP-941) applies to this data. The minor error associated with this measurement can occur because of a variety of factors including: (a) sample preparation (e.g. sample height), (b) instrument characteristics, (c) instrument calibration, (d) operator input (e.g. in determining the peak locations), and (e) the nature of the material (e.g. preferred orientation and transparency effects).

To obtain the absolute peak positions, the powder pattern should be aligned against a reference. This could either be the simulated powder pattern from the crystal structure of the same form solved at room temperature, or an internal standard (e.g. silica or corundum). The collected powder pattern of Form 3 was aligned to the simulated powder pattern.

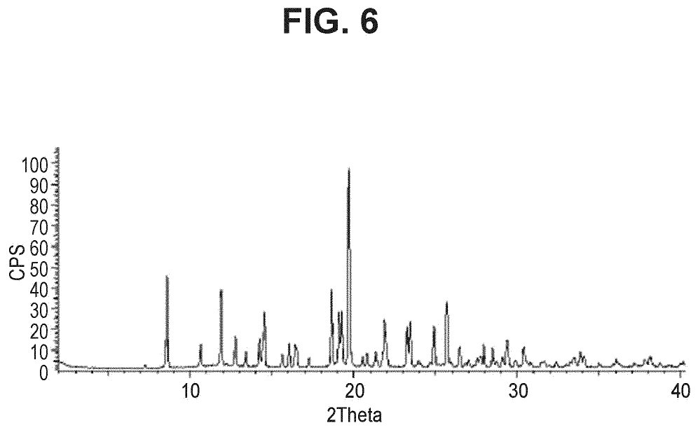

The PXRD profile for the N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide, hydrate (Form 3) is provided in FIG. 6 and the corresponding peak list is provided in Table PXRD1. Characteristic peaks for Form 3 are peaks at 8.6, 11.9, 14.6, 18.7, 19.7°2θ positions, each peak±0.2°2θ. Reference PXRD patterns of Form 1 and Form 2 are provided in FIGS. 7 and 8, respectively.

TABLE PXRD1
PXRD peak list for Form 3 (in degrees 2-theta,
each peak ±0.2 degrees 2-theta)

| Angle | | Angle | | Angle | |
|---|---|---|---|---|---|
| 2-Theta° | | 2-Theta° | | 2-Theta° | |
| ± | Relative | ± | Relative | ± | Relative |
| 0.2 | Intensity, | 0.2 | Intensity, | 0.2 | Intensity, |
| 2-Theta^{o} | % | 2-Theta° | % | 2-Theta^{o} | % |
| 8.6 | 46.7 | 18.7 | 38.9 | 24.9 | 20.5 |
| 10.7 | 11.6 | 19.1 | 27.7 | 25.7 | 32.9 |
| 11.9 | 38.7 | 19.3 | 28.1 | 26.5 | 9.7 |
| 12.8 | 15.1 | 19.7 | 100.0 | 27.8 | 5.1 |
| 13.4 | 7.8 | 20.6 | 5.0 | 28.5 | 9.4 |
| 14.3 | 14.2 | 20.8 | 6.5 | 29.4 | 13.4 |
| 14.6 | 27.9 | 21.4 | 7.3 | 30.4 | 10.2 |
| 15.6 | 6.3 | 21.9 | 23.9 | 33.9 | 7.8 |
| 16.1 | 12.0 | 23.3 | 20.1 | 34.1 | 5.3 |
| 16.4 | 11.2 | 23.5 | 22.9 | 38.1 | 5.1 |

### Solid State NMR

Solid state NMR (ssNMR) analysis was conducted on a Bruker-BioSpin Avance Neo 400 MHz (¹H frequency) NMR spectrometer. The ¹³C ssNMR spectrum was collected on a 4 mm MAS probe at a magic angle spinning rate of 15 kHz with the temperature was regulated to 25° C. A ¹³C cross-polarization (CP) spectra were recorded with a 2.5 ms CP contact time and recycle delay of 30 seconds. A phase modulated proton decoupling field of ˜100 kHz was applied during spectral acquisition. Carbon spectral referencing is relative to neat tetramethylsilane, carried out by setting the high-frequency signal from an external sample of α-glycine to 176.5 ppm.

Automatic peak picking was performed using ACD Labs 2019 Spectrus Processor software with a threshold value of 3% relative intensity used for preliminary peak selection. The output of the automated peak picking was visually checked to ensure validity and adjustments were manually made if necessary. Although specific ¹³C ssNMR peak values are reported herein there does exist a range for these peak values due to differences in instruments, samples, and sample preparation. A typical variability for ¹³C chemical shift x-axis values is on the order of plus or minus 0.2 ppm for a crystalline solid. The ssNMR peak heights reported herein are relative intensities. The ssNMR intensities can vary depending on the actual setup of the experimental parameters and the thermal history of the sample.

TABLE ssMR1
ssNMR peak list for Form 2

| Peak | Relative | Peak | Relative | Peak | Relative |
|---|---|---|---|---|---|
| (ppm) | Intensity, % | (ppm) | Intensity, % | (ppm) | Intensity, % |
| 20.4 | 109 | 54.7 | 120 | 128.7 | 46 |
| 22.0 | 108 | 55.5 | 86 | 138.3 | 46 |
| 24.1 | 50 | 66.2 | 72 | 154.6 | 100 |
| 29.6 | 35 | 98.8 | 77 | 162.2 | 41 |
| 34.6 | 27 | 102.6 | 55 | 174.5 | 45 |
| 38.3 | 46 | 106.6 | 61 | 182.8 | 47 |
| 39.0 | 49 | 119.8 | 75 | 211.2 | 95 |
| 41.3 | 25 | 123.3 | 68 | | |

### Formulation Examples of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide

N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide (hereinafter referred to as PF-00835231) is moderately lipophilic, with limited aqueous solubility. PF-00835231 is neutral throughout a physiologically relevant pH range and thus has pH-independent solubility behavior. The physicochemical properties of PF-00835231 limit the approaches that can be applied to improve its solubility for parenteral administration.

Due to limited aqueous solubility, low permeability, and short half-life, PF-00835231 is best suited for parenteral administration via intravenous (IV) infusion as an aqueous solution. Representative examples of aqueous solutions for infusion include a pharmaceutical composition with USP Water for Injection, 0.9% w/v sodium chloride, 5% w/v dextrose, 0.9% w/v sodium chloride in 5% w/v dextrose and lactated Ringer's solution. The predicted efficacious daily dose of PF-00835231 via continuous IV infusion is expected to be in the range of approximately 300 mg to 3300 mg. Daily continuous infusion volumes of about 250 mL to about 500 mL are typically preferred in order to stay above “keep vein open” (KVO) practices for minimum infusion rates used at many hospitals. Daily continuous IV infusion volumes up to about 1000 mL may be considered, but this large amount of fluid can limit co-administration of additional fluids. Based on the expected dose range and preferred IV infusion volumes, a target IV infusion concentration was about 1.2 mg/mL to about 13.2 mg/mL for a 250 mL IV infusion volume, about 0.6 mg/mL to about 6.6 mg/mL for a 500 mL IV infusion volume and about 0.3 mg/mL to about 3.3 mg/mL for the IV infusion volume of 1000 mL.

To achieve an IV infusion concentration of about 0.3 mg/mL to about 13.2 mg/mL, multiple solubilization approaches were considered. Typical solubilization approaches for IV pharmaceutical compositions include aqueous pH adjustment, salt form modification, co-solvent solubilization, surfactant solubilization, and complexation. In the case of PF-00835231, the neutral charge provides limited opportunities for pH adjustment or salt form modification, and thus solubilization excipients were evaluated. Solubilization excipients should be formulated at levels that are safe, and ideally, that are precedented by the same route of administration in order to minimize any possible adverse effects on the patient.

To improve the solubility of compounds with poor aqueous solubility, a mixture of solvents is often used. As used herein co-solvents can be defined as non-aqueous, water miscible solvents applicable for pharmaceutical use via parenteral administration. Representative examples of co-solvents include, but are not limited to, benzyl alcohol (BA), dimethylacrylamide (DMA), dimethyl sulfoxide (DMSO), ethanol, N-methyl pyrrolidone (NMP), polyethylene glycol (e.g. PEG200, PEG300, PEG400, PEG600), and propylene glycol (PG). The co-solvent method of solubilization can enable improvements in solubility by multiple orders of magnitude and has been successfully used in commercial products across multiple routes of administration to achieve higher dose levels. However, the co-solvent method of solubilization has multiple limitations for IV administration. First, the solvents used must be administered at levels that do not cause local irritation, systemic toxicity, or other adverse effects. These safety considerations often limit the upper concentrations that can be administered, especially in ready-to-use (RTU) IV formulations. Second, the solvents should be compatible with the drug product packaging and administration sets, and should not react, degrade, or extract any undesirable compounds. Third, an exponential relationship is expected between co-solvent content and drug solubility. Consequently, if the drug product is prepared as a powder or a concentrated ready-to-dilute (RTD) formulation, then dilution for IV administration can cause an exponential decrease in solubility that results in drug precipitation. Careful attention must thus be paid to the physical stability of the diluted formulation.

An alternative or additive approach to improve the solubility of lipophilic compounds involves the use of surfactants or polymeric excipients. Representative examples of surfactants include, but are not limited to, polyvinylpyrrolidone (PVP), poloxamer 407, poloxamer 188, hydroxypropyl methylcellulose (HPMC), polyethoxylated castor oil, lecithin, polysorbate 80 (PS80), polysorbate 20 (PS20) and polyethylene glycol (15)-hydroxystearate. Surfactants are typically amphiphilic molecules that self-assemble above a critical micelle concentration (CMC) to form a micelle, where the CMC and the structure that form are dependent on the composition of the formulation. In aqueous solutions, micelles typically orient the hydrophilic region of the molecule out into the aqueous solution and the lipophilic region of the molecule within the internal cavity of the micelle to limit interaction with water. In such micelles, amphiphilic and lipophilic compounds may be solubilized in the micelle wall or internal cavity, respectively, resulting in improved drug solubility. In the presence of co-solvents or oils, co-solvents can form oil-in-water emulsions that are stabilized by the surfactants. Like co-solvent only formulations, surfactant-based formulations have many of the same safety and compatibility constraints. In contrast to co-solvent only systems, surfactant-based formulations do not have the same exponential relationship between surfactant concentration and drug solubility. Instead, drug solubility is typically maintained for surfactant concentrations above the CMC. Consequently, surfactant-based formulations typically exhibit reduced precipitation upon dilution for IV administration.

Complexing agents are an alternative solubilization approach, where a substrate (i.e. a drug) forms a favorable non-covalent interaction with one or more ligands (i.e. complexing agents). Representative examples of complexing agents include, but are not limited to, cyclodextrins (CDs), hydrotropes, amino acids, or polymers. The most common class of complexing agents are cyclodextrins (CDs). CDs, in particular, are cyclic oligosaccharides with a variable number of D-glucose units (e.g. 6 for α-CD, 7 for β-CD, or 8 for γ-CD), and variable substitutions at the hydroxyl groups (e.g. hydroxypropyl, HP, or sulfobutylether, SBE). The CD shape provides a lipophilic cone-shaped cavity that can accommodate lipophilic drugs, where the number of D-glucose units modifies the cavity size and the substitutions modify the solubility of the complex and the favorability of complexation. CDs have several advantages over co-solvent or surfactant-based solubilization approaches, which typically include reduced toxicity, reduced container closure compatibility concerns, and improved manufacturability. Complexation with CDs is often more robust to dilution than co-solvent based solubilization, resulting in improved physical stability. However, CD complexation is limited to lipophilic molecules that can fit into the CD cavity and form favorable complexes. In addition, CD complexation can often require large CD quantities to enable significant solubility enhancement, which may lead to adverse effects. In addition, formation of the initial CD complex may be limited by the dissolution of the drug, which may ultimately limit the ability to solubilize the drug.

To enhance drug solubilization with CDs, researchers have investigated adding polymeric excipients to form ternary complexes. In particular, water-soluble polymers, including hydroxypropyl methylcellulose (HPMC), polyvinylpyrrolidone (PVP), and high molecular weight polyethylene glycols (PEGs) have been shown to enhance the drug dissolution rate of drugs and enable improved complexation with CDs.

In contrast to polymeric excipients, the use of co-solvents with CDs often results in decreased drug solubility as compared to CDs alone. In the textbook Water-Insoluble Drug Formulation, Tong and Wen summarized the primary issue: “One major problem with using organic co-solvents is that most organic co-solvents will compete for inclusion in the CD cavity, and thus inhibit complex formation.” Tong, W.-Q.; Wen, H; Application of Complexation in Drug Development for Insoluble Compounds in Liu, R. (Ed.) Water Insoluble Drug Formulation, CRC Press, Boca Raton, 2018, pp 149-176. An alternative hypothesis for decreased CD solubilization in the presence of co-solvents is that the co-solvent decreases the polarity of the aqueous medium, and thus reduces the driving force for lipophilic drugs to enter the lipophilic CD cavity. Multiple literature studies support this conclusion. See for example: P. Li, L. Zhao, S. H. Yalkowsky, Combined effect of cosolvent and cyclodextrin on solubilization of nonpolar drugs, Journal of Pharmaceutical Sciences, 88 (1999) 1107-1111; H. Viernstein, P. Weiss-Greiler, P. Wolschann, Solubility enhancement of low soluble biologically active compounds—temperature and cosolvent dependent inclusion complexation, International journal of pharmaceutics, 256 (2003) 85-94; Y. He, P. Li, S. H. Yalkowsky, Solubilization of Fluasterone in cosolvent/cyclodextrin combinations, International journal of pharmaceutics, 264 (2003) 25-34; T. Loftsson, B. J. Ólafsdóttir, H. Friõriksdóttir, S. Jónsdóttir, Cyclodextrin complexation of NSAIDSs: physicochemical characteristics, European Journal of Pharmaceutical Sciences, 1 (1993) 95-101; and J. Pitha, T. Hoshino, Effects of ethanol on formation of inclusion complexes of hydroxypropylcyclodextrins with testosterone or with methyl orange, International journal of pharmaceutics, 80 (1992) 243-251. For example, Viernstein et al. studied the fungicide triflumizole, a poorly water soluble compound, and found that: “generally, the combination of cosolvents and @-cyclodextrin does not increase the solubility of the compound, because cosolvents destabilize the inclusion complex.” Loftsson, et al. studied multiple non-steroidal anti-inflammatory drugs (NSAIDs) with poor aqueous solubility and found that: “all the NSAIDS tested formed inclusion complexes with the CDs, but addition of ethanol or propylene glycol to the aqueous CD solutions reduced their degree of complexation.”

Surprisingly, we find that complexing agents can be used to improve the aqueous solubility of PF-00835231 to enable the low end of the target solubility range in parenterally suitable compositions. Preferred complexing agents include CDs, amino acids and hydrotropes; more preferred complexing agents include β-CDs, γ-CDs, nicotinamide, sodium benzoate and sodium salicylate; most preferred complexing agents include HP-β-CD and SBE-β-CD. Surprisingly, β-CDs can form a complex with PF-00835231, despite the drug's moderate lipophilicity. A preferred embodiment of complexing agent-based pharmaceutical compositions of PF-00835231 is to formulate as a solution, which can then be sterile filtered, filled into an appropriate container closure system, and supplied as a solution. The solution can be supplied as an RTU solution that does not require further dilution prior to IV administration or as an RTD solution that does require dilution prior to IV administration. An additional preferred embodiment of complexing agent-based pharmaceutical compositions of PF-00835231 is to formulate as a solution, which can then be sterile filtered, filled into an appropriate container closure system, and freeze-dried to manufacture a lyophile. The lyophilized product may be reconstituted and/or diluted prior to IV administration.

Even more surprising, the solubility of PF-00835231 increases in formulations that contain one or more complexing agents and one or more co-solvents, which enables greater coverage of the target dose range. This finding is surprising due to previous reports that many co-solvents can have a destabilizing effect on complexation, as described above. Preferred complexing agents include CDs and hydrotropes; more preferred complexing agents include β-CDs, γ-CDs, nicotinamide, sodium benzoate and sodium salicylate; and most preferred complexing agents include HP-β-CD and SBE-β-CD. The amount of CDs in the final drug product (and in the aqueous liquid composition for IV administration), based on the molar ratio of CD to PF-00835231, is preferably in the range of about 1.5:1 to about 25:1, more preferably in the range of about 1.5:1 to about 8:1, and most preferably in the range of about 2:1 to 6:1. Preferred co-solvents may include one or more water-miscible polar protic and aprotic solvents, more preferred co-solvents may include dimethylacrylamide (DMA), N-methyl pyrrolidone (NMP), and benzyl alcohol (BA), and most preferred co-solvents include ethanol, propylene glycol (PG), dimethyl sulfoxide (DMSO) and polyethylene glycol (e.g. PEG200, PEG300, PEG400, PEG600). In some formulations, a combination of co-solvents is preferable to reduce the solution viscosity and limit any physical instabilities associated with solvent interactions. The most preferred two co-solvents in the final drug product (and in the aqueous liquid composition for IV administration), are one of ethanol and dimethyl sulfoxide (DMSO), and the other co-solvent is selected from the group consisting of PEG300, PEG400 and propylene glycol, wherein the ratio of ethanol or DMSO to PEG300, PEG400 or PG is preferably in the range of about 1:1 to about 1:9, and most preferably in the range of about 1:2 to about 1:4. The amount of total co-solvents in the final formulation (and in the aqueous liquid composition for IV administration), based on the volume fraction, is preferably up to about 15% v/v, and most preferably up to about 6% v/v. A preferred embodiment of complexing agent and co-solvent-based formulations of PF-00835231 is to formulate as a solution, where PF-00835231 is first solubilized in one or more co-solvents and subsequently combined with an aqueous mixture preferably containing a complexing agent. Surprisingly, the magnitude of solubility improvement is impacted by this order of excipient addition. A preferred embodiment of complexing agent and co-solvent-based formulations of PF-00835231 is to formulate as a solution, which can then be sterile filtered, filled into an appropriate container closure system, and supplied as a solution. The solution can be supplied as an RTU solution that does not require further dilution prior to IV administration or as an RTD solution that does require dilution prior to IV administration. An alternative preferred embodiment of complexing agent and co-solvent-based formulations of PF-00835231 is to formulate as a solution, which can then be sterile filtered, filled into an appropriate container closure system, and freeze-dried to manufacture a lyophile. The lyophilized product may be reconstituted and/or diluted prior to IV administration. An alternative embodiment of complexing agent and co-solvent-based formulations of PF-00835231 is to supply PF-00835231 as a powder in an appropriate container closure system, with at least two specialty diluents that contain the desired co-solvents and the desired surfactants, respectively. Examples of the PF-00835231 powder could be either sterile crystallized material or prepared by freeze drying. In this embodiment, the powder and diluents can be mixed in the appropriate order to produce an RTU or RTD product, which can be further diluted analogous to other embodiments. An alternative embodiment of complexing agent and co-solvent-based formulations of PF-00835231 is to formulate as a concentrated co-solvent solution, which can be sterile filtered, filled into an appropriate container closure system, and supplied as an RTD solution with a specialty diluent containing the desired solubilizing agents.

We also find that the solubility of PF-00835231 can also be increased in formulations that contain one or more surfactants and one or more co-solvents, which enables greater coverage of the target dose range than complexing agents alone, but less coverage than complexing agents and co-solvents. Preferred co-solvents may include water-miscible polar protic and aprotic solvents, more preferred co-solvents may include PG, DMA, and most preferred co-solvents include BA, DMSO, ethanol, NMP and polyethylene glycol (e.g. PEG300, PEG400). The preferred amount of co-solvent in the aqueous liquid composition for IV administration is up to about 30% v/v, more preferred amounts of co-solvents is up to about 20% v/v, and most preferred amounts of co-solvents is up to about 10% v/v. Preferred surfactants include non-ionic polymers, ionic polymers and lipids, more preferred surfactants include PVP, poloxamer 407, poloxamer 188, HPMC, polyethoxylated castor oil, lecithin, and most preferred surfactants include polysorbate 80 (PS80), polysorbate 20 (PS20), and polyethylene glycol (15)-hydroxystearate. The preferred amount of surfactant in the aqueous liquid composition for IV administration is up to about 100 mg/mL, and most preferred amount of surfactant is up to about 12.5 mg/mL. A preferred embodiment of surfactant and co-solvent-based pharmaceutical compositions of PF-00835231 is to formulate as a solution, which can then be sterile filtered, filled into an appropriate container closure system, and supplied as a solution. The solution can be supplied as an RTU solution that does not require further dilution prior to IV infusion or as an RTD solution that does require dilution prior to IV infusion. An additional preferred embodiment of a surfactant and co-solvent-based pharmaceutical composition of PF-00835231 is to formulate as a solution, which can then be sterile filtered, filled into an appropriate container closure system, and freeze-dried to manufacture a lyophile. The lyophilized product may be reconstituted and/or diluted prior to IV infusion. An additional preferred embodiment of a surfactant and co-solvent-based pharmaceutical composition of PF-00835231 is to supply PF-00835231 as a powder in an appropriate container closure system, with a specialty diluent that contains the desired co-solvents and the desired surfactants. In this embodiment, the powder and diluents can be mixed in the appropriate order to produce a RTU or RTD product, which can be further diluted analogous to other embodiments. The powder can comprise the Form 3 hydrate of PF-00835231.

In each of the preferred embodiments described above where the drug product is formulated as a solution, pH-dependent degradation is observed. To maximize drug stability, the final pharmaceutical composition preferably has an apparent pH in the range of about 2 to about 6, most preferably about 3 to about 5. In order to maintain the required pH, the pharmaceutical composition is buffered, with preferred buffers being acetic acid, lactic acid, phosphoric acid and tartaric acid, with the most preferred buffer being citric acid. Surprisingly, the combination of pH adjustment and CDs results in the most preferable chemical stability.

In each of the preferred embodiments described above where the drug product is lyophilized, a bulking agent, tonicity modifier, or water scavenging excipient may also be included. Preferred excipients include sugars, polyalcohols, polymers, and amino acids, more preferred excipients include PVP, sucrose, mannitol, lactose, and glycine, and most preferred excipients include trehalose, dextran, and low or high molecular weight PEGs.

### General Methodologies for Examples

### Ultra-Performance Liquid Chromatography (UPLC) Assay Method

A Waters Acquity UPLC system equipped with a Quaternary Solvent Manager, Sample Manager, Column Manger and a TUV detector equipped with an analytical flow cell detector (detection wavelength of 292 nm). A Cortecs T3, 1.6 μm, 2.1 mm×100 mm column was set at a temperature of 40±2° C. An injection volume of 1 μL was set to run at a flow rate of 0.3 mL/min. Mobile Phase A (10 mM Ammonium Formate, pH 3.0) and Mobile Phase B (Methanol) gradient was used to achieve the desired separation. The Mobile Phase A: Mobile Phase B ratio at 95:5 was set for 0 to 1 mins, the ratio was then set to 5:95 at 15 min mark, maintained same ratio until 16 min followed by 95:5 at 16.10 min and ran for 20 min at the same ratio.

### Ultra-Performance Liquid Chromatography (UPLC) Purity Method

A Waters Acquity UPLC system equipped with a Quaternary Solvent Manager, Sample Manager, Column Manger and a PDA detector (detection wavelength of 292 nm). A Kinetex F5, 1.7 μm, 2.1 mm×150 mm column was set at a temperature of 60±2° C. An injection volume of 5 μL was set to run at a flow rate of 0.4 mL/min. Mobile Phase A (20 mM Ammonium Formate, pH 3.0) and Mobile Phase B (20 mM Ammonium Formate in Methanol) gradient was used to achieve the desired separation. The Mobile Phase A: Mobile Phase B ratio at 65:35 was set for 0 to 1 mins, the ratio was then set to 45:55 at 31 min mark, followed by 5:95 at 46 min and set to 65:35 at 46.5 min and ran for 56 min.

### Powder X-Ray Diffraction Method 1

Powder X-Ray Diffraction (PXRD) was performed by loading approximately 20 mg of PF-00835231 sample in the holder. The measurement was performed on a Miniflex-600 using a Rigaku 906163 with a 10 mm×0.2 mm well sample holder. The system used a Rigaku PDXL2 software (V 2.8.4.0) and a Miniflex Guidance (V 3.2.20). The system was set in step mode and run from a starting degree of 2°2θ and a stop degree of 40°2θ with a step of 0.019°2θ for a duration of 1 second with a voltage of 40V and a current of 15 mA.

### Powder X-Ray Diffraction Method 2

The powder X-ray diffraction pattern was generated using a Bruker AXS D8 Endeavor diffractometer equipped with a Cu radiation source. The tube voltage and amperage were set to 40 kV and 40 mA, respectively. The motorized divergence slits were set at constant illumination of 11 mm. Diffracted radiation was detected using a LYNXEYE XE-T energy dispersive X-ray detector, with the position sensitive detector (PSD) opening set at 4.00°. Data was collected on the theta-theta goniometer at the Cu wavelength from 2.0 to 55.0°2θ using a step size of 0.019°2θ and a time per step of 0.1 seconds. Samples were prepared for analysis by placing them in a silicon low background holder and rotated at 15 rpm during data collection. Data were analysed in DIFFRAC.EVA software.

### Citrate Buffer Preparation

100 mL of 50 mM citrate buffer solutions were prepared in volumetric flasks from purified water, anhydrous citric acid, and sodium citrate dihydrate to target pH values of 3.0, 5.0, and 7.0. Buffer solutions were adjusted with 1 N sodium hydroxide or 1 N hydrochloric acid to reach the target pH.

For example, a 50 mM citrate buffer at pH 5 was prepared by first adding approximately 25 mL of purified water into a 100 mL volumetric flask. To this flask, approximately 331 mg of anhydrous citric acid (Sigma Aldrich, Ph. Eur/USP) and approximately 963 mg of trisodium citrate dihydrate (Sigma Aldrich, Ph. Eur./USP) were added. Purified water was added to the volumetric flask to the target volume and inverted to mix until homogeneous. The pH was measured, and no further pH adjustment was required.

### Formulation Example F1: PF-00835321 Aqueous Solubility

### Saturated Solubility Measurements

After preparation of the citrate buffer solutions, 1000 μL of buffer solution of the required pH was added to a clear Eppendorf tube. Approximately 7 mg of the hydrate form of PF-00835231 was then added to the citrate buffer solution in the Eppendorf tube. This process was repeated for a total of 3 replicates at each pH value. The solutions were observed to ensure that the PF-00835321 was not fully dissolved (i.e. the solution was saturated). The tubes were then sealed with parafilm, put inside dram vials and placed on roller mixers in a controlled temperature environment at approximately 4° C.

After 48 hours, the formulations were removed from the roller mixers and the pH was checked and found to be consistent with original value. Samples were then transferred to a new Eppendorf tube with a centrifuge filter. Formulations were then centrifuged for 3 minutes at 13,000 revolution per minute (rpm) at approximately 4° C. The solid retentate sample collected in the filter was analyzed via PXRD and the filtrate sample that passed through the filter was analyzed via UPLC (see above). The results from these saturated solubility experiments are found in Formulation Table F1 below, reported as average values.

FORMULATION TABLE F1
Data from aqueous saturated solubility
experiments for PF-00835231 at 4° C..
Solubility data is reported as an average of 3 replicates.

| | PF-00835231 Saturated | Solid Form |
|---|---|---|
| PH | Solubility at 4° C. (mg/mL) | at 4° C. |
| 3 | 0.1 | No Change-Hydrate |
| 5 | 0.1 | No Change-Hydrate |
| 7 | 0.1 | No Change-Hydrate |

These saturated solubility data confirm that PF-00835231 has poor aqueous solubility, independent of pH from 3.0 to 7.0, and will thus require the use of solubility-enabling formulation approaches in order to achieve a suitable dose in a volume of up to about 1000 mL.

### Formulation Example F2: PF-00835231 Solubility in Co-Solvent/Water Mixtures

### Saturated Solubility Measurements

The saturated aqueous solubility of PF-00835231 in co-solvent/water mixtures was investigated up to 25% v/v co-co-solvent content in water.

For each formulation, co-solvent stock solutions were first prepared at concentrations of 2.5% v/v, 10% v/v, or 25% v/v. To prepare the 25% v/v co-solvent stock solutions, 5 mL of co-solvent was added to a 20 mL volumetric flask, followed by 1 mL of 50 mM citrate buffer at pH 5, followed by water added to volume. To prepare the 10% v/v co-solvent stock solutions, 1 mL of co-solvent was added to a 10 mL volumetric flask, followed by 1 mL of 50 mM citrate buffer at pH 5.0, followed by water added to volume. To prepare the 2.5% v/v co-solvent stock solutions, 1 mL of the 25% v/v co-solvent stock solution was added to a 10 mL volumetric flask, followed by 0.9 mL of 50 mM citrate buffer at pH 5.0, followed by water added to volume.

2000 μL of the buffered co-solvent stock solutions was then added to HPLC vials. Approximately 10 mg of the hydrate form of PF-00835231 was then added to the solution in the HPLC vial. The solutions were mixed via vortexing for approximately 1 minute and formulations were observed to ensure that the PF-00835231 was not fully dissolved (i.e. the solution was saturated). The tubes were then sealed with parafilm and placed in a temperature-controlled incubator and rotated to mix. The temperature-controlled incubator was controlled to 40° C. for 8 hours, 15° C. for 5 hours, and 25° C. for 12 hours. At the conclusion of the experiment, the formulations were removed from the incubators and transferred to a new Eppendorf tube with a 0.2 μm PVDF centrifuge filter. Solutions were then centrifuged for 3 minutes at 13,000 rcf. The filtrate sample that passed through the filter was analyzed via HPLC. Solubility data are shown in

### Table 2.

FORMULATION TABLE F2
Saturated solubility data for PF-00835231 in mg/mL at
25° C. in the presence of varying amounts of co-solvent.
NC indicates that the formulation was not prepared at that
concentration. *indicates that saturation was
not achieved with the experiment.

| | Co-Solvent Concentration | | |
|---|---|---|---|
| Co-Solvent | 2.5% v/v | 10% v/v | 25% v/v |
| BA | NC | >5* | NC |
| DMSO | 0.2 | 0.4 | 1.5 |
| Ethanol | 0.2 | 0.4 | 1.6 |
| NMP | NC | 1.4 | NC |
| PEG300 | NC | 0.5 | NC |
| PEG400 | 0.2 | 0.5 | 1.5 |
| PG | 0.2 | 0.3 | 0.7 |

These solubility data demonstrate an exponential relationship between co-solvent concentration and saturated solubility. Of the tested co-solvents, they can be rank ordered as: PG<DMSO, Ethanol, PEG300, PEG400<NMP<BA. The most surprising aspects of these results are that PG improves solubility the least (˜50% of DMSO, Ethanol, PEG300, and PEG400 at 10% and 25% v/v) and that NMP and BA improve the solubility the most (˜3× and ˜10×, respectively, as compared to DMSO, Ethanol, PEG300, and PEG400 at 10% v/v). NMP may not be suitable for intravenous administration due to limited precedence and reports of toxicity via this route of administration, but may be suitable if the compound were to be administered via another route of administration (i.e. subcutaneous). For the 4 co-solvents that behaved comparably, modest improvements in solubility to ˜1.5 mg/mL were observed upon addition of 25% v/v of co-solvent. When compared to the target infusion concentration range for PF-00835231 of 0.3 mg/mL to 13.2 mg/mL, this data shows that a single co-solvent up to 25% v/v can only cover the low end of the target dose range.

Consequently, mixtures of different co-solvents were investigated to see if this could improve the solubility further, while reducing the excipient levels required.

### Formulation Example F3: PF-00835231 Solubility in Co-solvent/Co-solvent/Water Mixtures

### Saturated Solubility Measurements

The saturated aqueous solubility of PF-00835231 in co-solvent/co-solvent/water mixtures was investigated up to 50% v/v total co-solvent content in water that was adjusted to pH 5 and had 5 mM citrate buffer. Ethanol, PEG400, and PG were shortlisted as co-solvents due to their precedented use in IV administered products. 10 mL stock solutions were prepared at concentrations from 1% v/v to 25% v/v of each co-solvent, with 10% v/v of 50 mM citrate buffer adjusted to pH 5.0 and diluted to the target volume with water.

Approximately 10 mg of the hydrate form (PF-00835231 hydrate, Form 3) was weighed for each sample into an HPLC vial and 1 mL volume of co-solvent vehicle of interest was added. The process was completed for two replicates. The samples were vortexed and placed on a roller mixer in a 25° C. oven for 48 hours. After 48 hours, the samples were visually assessed to ensure that the PF-00835321 was not fully dissolved (i.e. the solution was saturated). The samples were transferred into a centrifuge plastic tube with a 0.2 μm PVDF centrifuge filter. The pH of the solutions was measured to confirm the pH had not changed after PF-00835231 addition. The samples were centrifuged 3 minutes at 13,000 rcf. The supernatant was collected and analysed by HPLC. Solubility data are shown in Formulation Table F3.

FORMULATION TABLE F3
Data from saturated solubility experiments for PF-00835231
at 25° C. in the presence of varying amounts of co-solvent.
Solubility data is reported as an average of 2 replicates.

| PEG400 | Ethanol | | PF-00835321 Saturated |
|---|---|---|---|
| (% v/v) | (% v/v) | PG (% v/v) | Solubility (mg/mL) |
| 1 | — | — | 0.1 |
| 0.75 | 0.25 | — | 0.1 |
| 1.88 | 0.63 | — | 0.2 |
| 3.75 | 1.25 | — | 0.2 |
| 2.5 | 2.5 | — | 0.2 |
| 7.5 | 2.5 | — | 0.5 |
| 5 | 5 | — | 0.4 |
| 7.5 | — | 2.5 | 0.3 |
| 11.25 | 3.75 | — | 0.6 |
| 10 | 10 | — | 1.1 |
| 10 | 25 | — | 1.9 |
| 25 | 10 | — | 1.8 |
| — | 25 | 25 | 4.6 |
| 25 | 25 | — | 7.7 |

These solubility data demonstrate a correlation between total co-solvent content and solubility, where PEG400 and ethanol achieve comparable solubilization, which is greater than PG. Modest improvements in solubility are observed up to 7.7 mg/mL in formulations containing 25% v/v of PEG400 and 25% v/v ethanol. When compared to the target infusion concentration range for PF-00835231 of 0.3 to 13.2 mg/mL, this data shows that two co-solvents up to 25% v/v each can cover over a greater portion, but not all of the target dose range. Consequently, mixtures of co-solvents and surfactants were investigated to see if this could improve the solubility further, while reducing the excipient levels required.

### Formulation Example F4: PF-00835231 Solubility in Co-Solvent/Co-Solvent/Surfactant Mixtures

Mixtures of co-solvents with surfactants were investigated to improve the solubility of PF-00835231 with reduced co-solvent levels and to reduce the risk of precipitation upon dilution.

### Saturated Solubility Measurements

The saturated aqueous solubility of PF-00835231 in co-solvent/co-solvent/surfactant mixtures was investigated. DMSO, Ethanol, PEG400, and PG were used as co-solvents. Polysorbate 80, polysorbate 20, and polyethylene glycol (15)-hydroxystearate were used as surfactants.

For each formulation, co-solvent/co-solvent/surfactant stock solutions were first prepared in volumetric flasks with either:
25% v/v co-solvent 1, 25% v/v co-solvent 2, and 12.5 mg/mL surfactant
10% v/v co-solvent 1, 10% v/v co-solvent 2, and 5 mg/mL surfactant
5% v/v co-solvent 1, 5% v/v co-solvent 2, and 2.5 mg/mL surfactant

All formulations were prepared with a final concentration of 5 mM citrate buffer at approximately pH 5.0. The selected concentrations bracket possible RTU and RTD formulations and help to map the possible formulation design space.

Approximately 20 mg of the hydrate form of PF-00835231 was added to each HPLC vial. 1.5 mL of the buffered co-solvent/co-solvent/surfactant stock solutions were then added to the HPLC vials. The solutions were mixed via vortexing for approximately 1 minute and formulations were observed to ensure that the PF-00835231 was not fully dissolved (i.e. the solution was saturated). The tubes were then sealed with parafilm and placed in a temperature-controlled incubator and rotated to mix. The temperature-controlled incubator was controlled to 40° C. for 8 hours, 15° C. for 5 hours, and 25° C. for 12 hours at 12 rpm rotation. At the conclusion of the experiment, the formulations were removed from the incubators and transferred to a new Eppendorf tube with a 0.2 m PVDF centrifuge filter. Solutions were then centrifuged for 3 minutes at 13,000 rcf. The filtrate sample that passed through the filter was analyzed via HPLC. Solubility data are shown in Formulation Table F4.

FORMULATION TABLE F4
Co-solvent/co-solvent/surfactant solubility data

| | Co- | | Co- | | Sur- | PF- |
|---|---|---|---|---|---|---|
| Co- | Solvent | Co- | Solvent | | factant | 00835231 |
| Solvent | 1 | Solvent | 2 | | (mg/ | Solubility |
| 1 | (% v/v) | 2 | (% v/v) | Surfactant | mL) | (mg/mL) |
| Ethanol | 5 | PEG400 | 5 | PS80 | 2.5 | 0.7 |
| Ethanol | 5 | PEG400 | 5 | PS20 | 2.5 | 0.6 |
| Ethanol | 5 | PEG400 | 5 | polyethylene | 2.5 | 0.6 |
| | | | | glycol (15)- | | |
| | | | | hydroxystearate | | |
| Ethanol | 5 | DMSO | 5 | PS80 | 2.5 | 0.6 |
| Ethanol | 5 | PG | 5 | PS80 | 2.5 | 0.6 |
| PEG400 | 5 | DMSO | 5 | PS80 | 2.5 | 0.7 |
| PEG400 | 5 | PG | 5 | PS80 | 2.5 | 0.6 |
| Ethanol | 10 | PEG400 | 10 | PS80 | 5 | 1.3 |
| Ethanol | 25 | PEG400 | 25 | PS80 | 12.5 | 16.6 |
| Ethanol | 25 | PG | 25 | PS80 | 12.5 | 12.5 |

These solubility data demonstrate an improvement in solubility for mixtures containing two co-solvents and a surfactant, as compared to two co-solvents alone (Formulation Example F3). The trends in co-solvent solubilities were consistent with the previous examples. All 3 surfactants used (PS20, PS80, and Kolliphor HS-15) achieved comparable solubility. From these results, a solubility of ˜16.6 mg/mL could be achieved upon addition of 25% v/v ethanol, 25% v/v PEG400, and 12.5 mg/mL PS80, which is ˜9 mg/mL greater than the same formulation without PS80. When compared to the target infusion concentration range for PF-00835231 of 0.3 mg/mL to 13.2 mg/mL, this data shows that this composition can cover the full target dose range. However, formulations with high co-solvent levels may present safety risks that would prevent these formulations from being used clinically to cover the high end of the target dose range. Consequently, formulations with lower levels of co-solvents may be required to cover the low end of the target dose range, or the formulations with higher levels of co-solvents may have to be diluted prior to administration.

### Solubility and Stability in Pure Co-Solvent and Surfactant Mixtures

To investigate the most concentrated options for co-solvent and surfactant containing formulations, formulations were prepared with pure co-solvent and surfactant. The vehicles of interest were prepared prepared by adding the required quantity of ethanol (Merck), PEG400 (Sigma Aldrich), and polysorbate 80 super refined (Croda) into a 25 mL volumetric flask and making up to volume with ethanol. Compositions are shown in Formulation Table. Approximately 30 mg of the hydrate form of PF-00835231 was added into an HPLC vial, followed by approximately 0.5 mL of the vehicle of interest. The samples were then placed on a roller mixer in the 25° C. oven. After 48 hours all solutions were clear, indicating that saturated solubility had not been achieved. Solutions were then centrifuged twice for 3 minutes at 13,000 rcf. The supernatant was collected and analyzed via HPLC. Because saturated solubility had not been achieved, the HPLC data is a minimum solubility achieved, as shown in Formulation Table F5.

FORMULATION TABLE F5
Data from saturated solubility experiments for PF-00835231 at 25°
C. in the presence of varying amounts of co-solvent and surfactant.
Solubility data is reported as an average of 2 replicates.

| | Ethanol | Polysorbate 80 | PF-00835321 Solubility |
|---|---|---|---|
| PEG400 (% v/v) | (% v/v) | (mg/mL) | (mg/mL) |
| 30 | 60 | 100 | >45 |
| 60 | 30 | 100 | >45 |
| 45 | 45 | 100 | >45 |

The data in Formulation Table F5 indicates that mixtures of pure co-solvent and surfactant can solubilize PF-00835231 above the target infusion concentration range of 0.3 mg/mL to 13.2 mg/mL. However, the concentrated co-solvent mixtures shown in Formulation Table F5 are likely not suitable for IV administration at the target infusion volumes of 250 mL to 500 mL due to the amount of co-solvent and surfactant present. Consequently, these formulations must be diluted in a relevant diluent (i.e. 0.9% w/v saline) and the resultant admixtures must be monitored for physical stability (i.e. whether the drug remains in solution or precipitates out).

FORMULATION TABLE F6
Vehicle mixtures of co-solvents and surfactant
to be used in dilution studies.

| Vehicle | Ethanol (% v/v) | PEG400 (% v/v) | Polysorbate 80 (mg/mL) |
|---|---|---|---|
| 1 | 45 | 45 | 67.5 |
| 2 | 60 | 30 | 100 |

The required quantities of excipients shown in Formulation Table F6 were weighed into a 50 mL volumetric flask. In order to control the final pH, 245 μL of 0.1 M citric acid anhydrous in ethanol was added to Vehicle 1 and 288 μL of 0.1 M citric acid anhydrous in ethanol was added to Vehicle 2. Vehicle 1 was made up to volume with purified water and Vehicle 2 was made up to volume with ethanol and stirred until mixed.

Approximately 800 mg of the hydrate form of PF-00835231 was added to 20 mL volumetric flasks and made to volume with either Vehicle 1 or Vehicle 2. A small magnetic flea was added, and the volumetric flasks were transferred onto a magnetic stirrer plate. The mixtures were stirred until a homogeneous solution of approximately 40 mg/mL was achieved. The formulations were then ready for dilution studies, where Vehicle 1 with PF-00835231 is described as Formulation 1 and Vehicle 2 with PF-00835231 is described as Formulation 2. Based on HPLC measurements the concentration of PF-00835231 was found to be approximately 36 mg/mL in Formulation 1 and approximately 37 mg/mL in Formulation 2.

Dilutions were performed in a laminar air-flow hood under aseptic conditions to minimise extrinsic particles. Glassware was pre-washed and rinsed 10 times with filtered water to reduce the visible and sub-visible particulates. Glassware was left to dry overnight in a laminar air flow cabinet before experiment. To perform the dilution, Formulation 1 and Formulation 2 were filtered via a 0.50 μm PVDF syringe filter into separate holding containers. For Formulation 1, approximately 5.56 mL was added to a 100 mL flask containing 0.9% w/v saline (1 in 18 dilution, to approximately 2 mg/mL). The entire 5.56 mL volume of Formulation 1 was added at once and then the flask was capped and mixed via inversion. This process was repeated for a total of 3 samples. For Formulation 2, approximately 4.17 mL was added to a 100 mL flask and diluted to 100 mL with saline (1 in 24 dilution to approximately 1.5 mg/mL). The entire 4.17 mL volume of Formulation 2 was added at once and then the flask was capped and mixed via inversion. This process was repeated for a total of 3 samples.

The flasks were left to stand at room temperature. Visual appearance assessment was completed on the volumetric flasks at approximately 5 minutes, 10 minutes, 30 minutes, 1 hour, 2 hours, 3 hours, 16 hours, 20 hours, and 24 hours, and compared to a placebo controls (placebo controls were Formulations 1 and 2 without PF-00835231 diluted in saline as described above). In all cases particles were observed in the volumetric flasks containing PF-00835231 at the 16-hour time point, indicating precipitation had occurred between 3 hours and 16 hours. The placebo controls were observed to be clear.

These data indicate that mixtures of pure co-solvents and surfactants can significantly improve the solubility of PF-00835231 to enable the low end of the target dose range. However, full coverage of the dose range is not possible due to the limited physical stability of higher concentration formulations upon dilution. Consequently, alternative formulations were evaluated.

### Formulation Example F5: PF-00835231 Solubility in CD/Water Mixtures

Due to the limited success with co-solvent and surfactant-based solubilization approaches, alternative approaches were evaluated. Specifically, SBE-β-CD and HP-β-CD were investigated as complexing agents.

### Saturated Solubility Experiments

The saturated solubility of PF-00835231 was evaluated in solutions adjusted to pH 5 with 5 mM citrate buffer, with CD concentrations that varied from 15 mg/mL to 100 mg/mL. The CD solution were prepared by adding 2.5 mL of a 50 mM citrate buffer at pH 5.0 to a 25 mL volumetric flask. The required quantity of SBE-β-CD (Carbonate, Pharma Grade) and HP-β-CD (Roquette, Parenteral Grade, EP-USP/NF), corrected for water content, was weighed into the volumetric flask. The volumetric flask was made up to volume with purified water, then stirred until fully dissolved and mixed.

Approximately 5 mg of the hydrate form of PF-00835231 was weighed and transferred into a 2 mL Eppendorf tube. 1 mL of CD solution was added, and the sample was vortexed. A suspension was obtained, and the sample was sonicated for 5 minutes. After sonication the samples were placed in appropriately labelled dram vials, and then kept on a roller mixer in the 25° C. oven for 48 hours. This process was repeated so that two samples are generated for each CD solution. The samples were observed after 48 hours. The samples were centrifuged for 3 minutes at 13,000 rpm in a centrifugal filtration device (0.22 μm PVDF filter). The filtrate was collected, and the pH was measured to confirm no change, and the sample was analysed by HPLC for potency.

FORMULATION TABLE F7
Data from saturated solubility experiments
for PF-00835231 in CD mixtures at 25° C.

| | | PF-00835231 Saturated |
|---|---|---|
| SBE-β-CD (mg/mL) | HP-β-CD (mg/mL) | Solubility (mg/mL) |
| 15 | — | 0.3 |
| 25 | — | 0.5 |
| 50 | — | 0.8 |
| 100 | — | 1.5 |
| — | 15 | 0.3 |
| — | 25 | 0.5 |
| — | 50 | 1.0 |
| — | 100 | 1.9 |

Based on the data in Formulation Table F7, CDs were able to improve the solubility of PF-00835231 to 1.5 mg/mL for SBE-β-CD and 1.9 mg/mL for HP-β-CD at a CD concentration of 100 mg/mL. When compared to the target infusion concentration range for PF-00835231 of 0.3 mg/mL to 13.2 mg/mL, this data shows that CDs up to 100 mg/mL can cover the lower end of the target dose range.

### Method for Room Temperature Roller Mix Experiment

A 100 mg/mL solution of HP-β-CD in 5 mM citrate buffer was prepared. Approximately 5 mg of the hydrate form of PF-00835231 was weighed into a 2 mL Eppendorf tube. 1 mL of CD in 5 mM citrate buffer solution was added into the 2 mL Eppendorf tube. The sample was vortexed then placed in appropriately labelled dram vials and kept on a roller mixer in the 25° C. oven for 48 hours. This process was repeated such that two samples were generated for each CD solution. Samples were observed after 48 hours and solid was remaining. The sample was then centrifuged for 3 minutes at 13,000 rpm in a centrifugal filtration device (0.22 μm PVDF filter). The supernatant was collected. The pH was measured, and the solid form of the residual solid was tested by PXRD. No changes in solid form were detected. The sample was analysed by HPLC for potency, results shown in Formulation Table F8 (Control with no sonication, roller mixer at 25° C. for 48 hrs.).

### Extended Sonication Experiment

A 100 mg/mL solution of HP-D-CD in 5 mM citrate buffer was prepared. Approximately 5 mg of the hydrate form of PF-00835231 was weighed into a 2 mL Eppendorf tube. 1 mL of CD in 5 mM citrate buffer solution was added into the 2 mL Eppendorf tube. The sample was vortexed and then sonicated for 20 minutes. The sample was then placed in dram vials and kept on a roller mixer in the 25° C. oven for 48 hours. This process was repeated such that two samples are generated for each CD solution. Samples were observed after 48 hours and solid were present. The sample was then centrifuged for 3 minutes at 13,000 rpm in a centrifugal filtration device (0.22 μm PVDF filter). The filtrate was collected, the pH was measured to confirm no change, and the filtrate was analysed by HPLC for potency. Results are shown in Formulation Table F8 (Sonication for 20 minutes, then roller mixer at 25° C. for 48 hrs.)

FORMULATION TABLE F8
Data from saturated solubility experiments for PF-00835231
in CD mixtures prepared via various methods of preparation.

| | | | PF-00835231 |
|---|---|---|---|
| SBE-β- | HP-β- | | Saturated |
| CD | CD | | Solubility |
| (mg/mL) | (mg/mL) | Method of Preparation | (mg/mL) |
| 100 | — | Heat at 40° C. for 24 hours, | 1.7 |
| | | then roller mixer at 25° C. | |
| | | for 48 hrs. | |
| — | 100 | No sonication, roller mixer at | 1.9 |
| | | 25° C. for 48 hrs. (control) | |
| — | 100 | Sonication for 20 minutes, | 1.9 |
| | | then roller mixer at 25° C. | |
| | | for 48 hrs. | |
| — | 100 | Heat at 40° C. for 24 hours, | 2.0 |
| | | then roller mixer at 25° C. | |
| | | for 48 hrs. | |

Method for Heat, then Roller Mixer Experiment

100 mg/mL solutions of SBE-β-CD and HP-β-CD were prepared in 5 mM citrate buffer. Approximately 5 mg of the hydrate form of PF-00835231 was weighed into a 2 mL Eppendorf tube. 1 mL of CD in 5 mM citrate buffer solution was added into the 2 mL Eppendorf tube. The sample was vortexed then placed in dram vials and kept on a roller mixer in a 40° C. oven for 24 hours. The sample was then placed on a roller mixer in a 25° C. oven for 48 hours. This process was repeated such that two samples are generated for each CD solution. The samples were then centrifuged for 3 minutes at 13,000 rpm in a centrifugal filtration device (0.22 μm PVDF filter). The filtrate was collected, the pH was measured to confirm no change, and the filtrate was analysed by HPLC for potency. The results are shown in Formulation Table F8 (Heat at 40° C. for 24 hours, then roller mixer at 25° C. for 48 hrs.). Based on the solubility data in Formulation Table F8, relative to Formulation Table F7, the solubility of PF-00835231 was not substantially impacted by heating or sonication.

When compared to the target infusion concentration range for PF-00835231 of 0.3 mg/mL to 13.2 mg/mL, this data shows that formulations containing CDs at concentrations of up to 100 mg/mL can only cover the lower portion of the target dose range. Consequently, mixtures of CDs with other excipients were investigated to see if this could improve the solubility further, while reducing the excipient levels required.

### Formulation Example F6: PF-00835231 Solubility in CD/Water Mixtures with Polymeric Excipients

### Saturated Solubility Experiments

The saturated solubility of PF-00835231 was evaluated in solutions adjusted to pH 5 with 5 mM citrate buffer, with CD concentrations fixed at 15 mg/mL. The effect of different polymeric excipients was investigated based on literature reports of enhanced solubilities when investigated together.

10 mL stock solutions for each formulation listed in Table 8 were prepared by mixing 1 mL of a 50 mM citrate buffer, 1 mL of 150 mg/mL HP-β-CD stock solution, and the target amount of polymeric excipients into a 10 mL volumetric flask. Stock solutions were diluted to the target volume with purified water. The following polymeric excipients were used: PEG400 (Fisher Chemical, NF), PEG3350 (Spectrum Chemical, USP), HPMC (Sigma, Viscosity 2600-5600 cP), and PVP (Alfa Aesar, Molecular weight 8000 Da).

Approximately 2.5 mg of the hydrate form of PF-00835231 was then added to an HPLC vial for each formulation and approximately 0.5 mL of the stock solution was added to create a saturated solution at approximately 5 mg/mL of PF-00835231. Formulations were sonicated for 5 minutes and then placed on a shaker for approximately 24 hours at ambient conditions. The formulations were then transferred to a centrifuge tube with a 0.1 m PVDF centrifugal filter and centrifuged at 13,000 rcf for 3 minutes. The filtrate was collected and analyzed via HPLC against a PF-00835231 standard to provide an assay value for PF-00835231.

FORMULATION TABLE F9
Data from saturated solubility experiments for PF-00835231 in CD
mixtures at 25° C. prepared with various polymeric excipients.

| HP-β-CD | | Polymeric Excipient | PF-00835231 |
|---|---|---|---|
| Concentration | Polymeric | Concentration | Assay |
| (mg/mL) | Excipients | (mg/mL) | (mg/mL) |
| 15 | — | — | 0.4 |
| 15 | PEG400 | 0.25 | 0.4 |
| 15 | PEG400 | 2.5 | 0.4 |
| 15 | PEG3350 | 0.25 | 0.4 |
| 15 | HPMC | 1 | 0.4 |
| 15 | PVP | 0.25 | 0.4 |

Based on the solubility data in Formulation Table F9, the solubility of PF-00835231 was not substantially impacted by the additive excipients investigated. Consequently, the clinical application of CD-based formulations is not improved with the additives investigated.

### Formulation Example F7: PF-00835231 Solubility in CD/Co-Solvent/Water Mixtures Prepared with Different Orders of Addition

### Solubility Experiment

The solubility of PF-00835231 was investigated in aqueous solutions containing ethanol and HP-β-CD. An approximately 300 mg/mL HP-β-CD stock solution was prepared by weighing approximately 6.00 g of HP-β-CD (Ashland, Pharma Grade) powder in a 20 mL volumetric flask and diluting to volume with purified water. The flask was capped and inverted to mix until clear. Subsequently, an approximately 15 mg/mL HP-β-CD stock solution was prepared by adding 0.5 mL of the approximately 300 mg/mL HP-β-CD stock solution and 1 mL of an approximately 50 mM citrate buffer adjusted to pH 5.0 to a 10 mL volumetric flask and diluting to volume with purified water.

PF-00835231 formulations were prepared in 4 mL vials by first adding approximately 8 mg of the hydrate form of PF-00835231 to the vial. In one instance, approximately 100 μL of ethanol (Pharmco-AAPER, ACS/USP Grade) was added to the PF-00835231, the solution was vortexed until dissolved, followed by 3.9 mL of the approximately 15 mg/mL HP-β-CD stock solution. The resultant formulation has a final composition of approximately 15 mg/mL HP-β-CD and 2 mg/mL PF-00835231, resulting in a CD:PF-00835231 molar ratio of approximately 2.5, and 2.5% v/v of co-solvent. In another instance, 100 μL of ethanol was added to PF-00835231, the solution was vortexed until dissolved, and 3.9 mL of a 5 mM citrate buffer solution at pH 5.0 was added. In another instance, 3.9 mL of the 15 mg/mL HP-β-CD stock solution was added to PF-00835231, the solution was vortexed until dissolved, and 100 μL of ethanol of was added.

Formulations were sonicated for approximately 3 minutes and then placed on a shaker for approximately 24 hours at ambient conditions. The formulations were then transferred to a centrifuge tube with a 0.1 μm PVDF centrifugal filter and centrifuged at 13,000 rcf for 3 minutes. The filtrate was collected and analyzed via HPLC against a PF-00835231 standard to provide an assay value for PF-00835231.

FORMULATION TABLE F10
Data from solubility experiments for PF-00835231 in
aqueous mixtures containing ethanol and HP-β-CD
at 25° C. prepared via different orders of addition.

| | | PF- | | | |
|---|---|---|---|---|---|
| | | 00835231 | | | |
| | | concen- | | Visual | PF- |
| | HP-β- | tration at | | Obser- | 00835231 |
| Ethanol | CD | Preparation | Order of | vation | Assay |
| (% v/v) | (mg/mL) | (mg/mL) | Addition | 24 hrs. | (mg/mL) |
| 2.5 | 15 | 2 | PF-00835231, | Clear | 1.9 |
| | | | Ethanol, | | |
| | | | HP-β-CD | | |
| | | | with buffer | | |
| 2.5 | 15 | 2 | PF-00835231, | Partic- | 0.4 |
| | | | HP-β-CD | ulate | |
| | | | with buffer, | | |
| | | | Ethanol | | |
| 2.5 | 0 | 2 | PF-00835231, | Partic- | 0.4 |
| | | | Ethanol, | ulate | |
| | | | Buffer | | |

Based on the solubility data in Formulation Table F10, the solubility of PF-00835231 was improved by approximately 5×from 0.4 mg/mL to 1.9 mg/mL if the order of addition for ethanol relative to CD is reversed. Specifically, if PF-00835231 is first dissolved in ethanol, followed by dilution with a CD-containing solution, this leads to improved solubility. The improved solubility data is further supported by visual observations, which show that the solution with lower solubility has visible particulate after 24 hours, while the solution with higher solubility is clear after 24 hours. A control experiment that first dissolves PF-00835231 in ethanol, followed by dilution without CD, shows that CD is required to achieve the target solubility and produce a physically stable formulation.

When the solubility data in Formulation Table F10 is compared to the target infusion concentration range for PF-00835231 of 0.3 mg/mL to 13.2 mg/mL, this data shows that HP-β-CD at approximately 15 mg/mL with ethanol at approximately 2.5% v/v could cover the lower end of the target dose range, but is insufficient to cover the full target dose range.

### Freeze-Thaw Experiment

After the solubility experiment, the solutions were placed in a −20° C. freezer for ˜24 hours, removed and placed at room temperature and allowed to thaw. The formulations were then analyzed via visual inspection. No particulates were observed in any sample, demonstrating that these formulations are physically stable to freezing.

### Formulation Example F8: PF-00835231 Solubility in CD/Co-Solvent/Water Mixtures Prepared with Different Compositions

To investigate whether alternative co-solvent combinations of PF-00835231 can produce comparable results, additional formulations were prepared. PF-00835231 was first solubilized in a fixed volume of one or two co-solvents. The co-solvent mixtures were subsequently combined with aqueous to produce pharmaceutical compositions with approximately 80 mg/mL SBE-β-CD, 5 mM citrate buffer, approximately 3.0% v/v and PF-00835231 concentrations of approximately 4 mg/mL, respectively. The target compositions have a CD:PF-00835231 molar ratio of approximately 4.2:1. These formulations would enable delivery of up to 1 g, 2 g, or 4 g dose of PF-00835231 in a 250 mL, 500 mL, or 1000 mL administration volume.

Specifically, a 10 mL stock solution of each co-solvent combination in Formulation Table F was prepared in 10 mL volumetric flasks through measuring 2.5 mL of co-solvent 1 followed by dilution to volume with co-solvent 2 to prepare stock solutions of approximately 75% co-solvent 1/25% co-solvent 2 by volume. Flasks were inverted several times to mix and placed in 40° C. oven for 30 approximately minutes prior to experiment. An approximately 300 mg/mL SBE-β-CD stock solution was prepared by weighing approximately 3.00 g of SBE-D-CD powder in a 10 mL volumetric flask and diluting to volume with purified water. The flask was capped and inverted to mix until clear.

PF-00835231 stock solutions were prepared in 2 mL HPLC vials by first adding approximately 20 mg of the hydrate form of PF-00835231 to the vial, followed by addition of approximately 150 μL of the heated co-solvent stock mixture. The solution was then vortexed to mix for ˜1 minute. The resultant PF-00835231 stock solution was placed in a temperature-controlled incubator at 40° C., where the samples were rotated to mix. Solution was removed after 20 minutes when fully dissolved.

In separate 2 mL HPLC vials, 0.15 mL of an approximately 50 mM citrate solution adjusted to pH 5.0 and 0.4 mL of 300 mg/mL SBE-β-CD stock solution were mixed. Subsequently, 0.905 mL of purified water was added to the vials. After this addition, 45 mL of PF-00835231 solution was then transferred to the CD-containing solutions to create 1.5 mL of approximately 4 mg/mL PF-00835231 solutions. The solutions were then capped and vortexed to mix. Solutions were then placed on a shaker at ambient conditions.

An aliquot of each formulation was removed after approximately 1 and 5 days for assay determination via HPLC. Specifically, 150 μL aliquots were added to a centrifugal filter with a 0.1 μm PVDF filter and centrifuged for 3 minutes at 13,000 rcf. The filtrate was collected and analyzed via HPLC against a PF-00835231 standard to provide an assay value for PF-00835231, which is reported in Formulation Table F11.

FORMULATION TABLE F11
Assay values of filtered PF-00835231
formulations over 5 days. The assay values do not change over
5 days, which reflects the physical stability of the
formulations. Potency was not corrected for impurities
and water content, which led to assay values below target. All
formulations prepared at pH 5.0, with 80 mg/mL SBE-β-
CD and 5 mM citrate buffer, at a target concentration of 4 mg/mL PF-00835231.

| Co- | Co- | Co- | Co- | | Assay | Assay |
|---|---|---|---|---|---|---|
| Solvent | Solvent | Solvent | Solvent | Polysorbate | (Day | (Day |
| 1 | 1 (% v/v) | 2 | 2 (% v/v) | 80 (mg/mL) | 1) | 5) |
| DMSO | 3.0 | — | — | — | 4.1 | 4.3 |
| PG | 3.0 | — | — | — | 3.8 | 3.8 |
| Ethanol | 0.75 | DMSO | 2.25 | — | 3.9 | 3.9 |
| Ethanol | 0.75 | PG | 2.25 | — | 3.8 | 3.8 |
| Ethanol | 0.75 | PEG300 | 2.25 | — | 3.8 | 3.7 |
| Ethanol | 0.75 | PEG400 | 2.25 | — | 3.6 | 3.6 |
| Ethanol | 0.75 | PEG400 | 2.25 | 2.5 | 3.6 | 3.5 |
| DMSO | 0.75 | PEG400 | 2.25 | — | 4.0 | 3.9 |

Formulation Table F11 demonstrates that one or more co-solvents can be used to solubilize PF-00835231, and the co-solvent mixtures can subsequently be combined with CD containing aqueous solutions to create physically stable formulations. All solutions remained clear after 5 days mixing at 25° C. Furthermore, consistent PF-00835231 assay values were observed between 1 and 5 days, within the error of the measurement. The data in Formulation Table F11 shows comparable physical stability of formulations prepared with the following co-solvents: DMSO alone, PG alone, combinations of ethanol with DMSO, PG, PEG300, or PEG400; or combinations of DMSO and PEG400.

### Formulation Example F9: Composition Optimization to Minimize Precipitation and Maximize Solubility and Stability

In Formulation Example F8, it was found that dissolution of PF-00835231 in co-solvent, followed by mixing with a CD containing solution resulted in improved solubility and physical stability. Upon further investigation of this experimental approach, PF-00835231 precipitated out of solution inconsistently upon addition to ethanol (prior to CD addition). To enable more consistent results with this experimental approach, and to reduce the likelihood of PF-00835231 precipitation, an additional co-solvent (PEG400) was added to ethanol. Data from over 50 different formulations was collected for PF-00835231 concentrations ranging from approximately 10 mg/mL to 240 mg/mL and volume fractions of PEG400 relative to ethanol ranging from 0% to 100%.

### Formulation Preparation

In a representative experiment, 20 mL stock solutions of co-solvent mixtures were prepared in 20 mL volumetric flasks through measuring 0 mL, 2 mL, 4 mL, 5 mL, 10 mL, 15 mL, or 20 mL of ethanol (Pharmco-AAPER, ACS/USP Grade) followed by dilution to volume with PEG400 (Fisher Chemical, Carbowax, NF Grade) to prepare stock solutions of approximately 100%, 90%, 80%, 75%, 50%, 25%, or 0% PEG400.

In a representative experiment, approximately 300 mg/mL SBE-β-CD or HP-β-CD stock solutions were prepared by weighing approximately 6.00 g of SBE-β-CD or HP-β-CD powder in 20 mL volumetric flasks and diluting to volume with purified water. The flask was capped and inverted to mix until clear.

In a representative experiment, PF-00835231 formulations were prepared in 2 mL HPLC vials by first adding a defined amount of the hydrate form of PF-00835231 to the vial, followed by addition of small amounts of a co-solvent stock mixture, typically 100 μL to 500 μL. The solution was then dissolved by vortexing and/or heating the solution to 40° C. until the solution was visibly clear. If precipitation was observed, the samples were not progressed further.

In a separate 2 mL HPLC vial, 100 μL of a 50 mM citrate solution adjusted to pH 5.0, a defined amount of 300 mg/mL SBE-β-CD or HP-β-CD stock solution, and a defined amount of purified water were mixed. The PF-00835231 solution in co-solvents was then mixed with the CD-containing solution. The combined solution was then capped and vortexed to mix. Solutions were then placed on a shaker for approximately 24 hours at ambient conditions. The formulations were then transferred to a centrifuge tube with a 0.1 μm PVDF centrifugal filter and centrifuged at 13,000 rcf for 3 minutes. The filtrate was collected and analyzed via HPLC against a PF-00835231 standard to provide an assay value for PF-00835231. Based on the data in 0, one can conclude that solutions with high volume fraction of ethanol (50% or greater) and high concentrations of PF-00835321 (above 100 mg/mL) tend to favor precipitation. Consequently, to limit the likelihood of precipitation and to enable complete solubilization of PF-00835231 in co-solvent mixtures, a volume fraction of at least 50% PEG400 should be utilized, preferably at least 75% PEG400. Under these conditions, PF-00835231 solubility of at least 200 mg/mL can be achieved. In addition, at 100% volume fraction of PEG400, the solutions become very viscous and dissolution proceeds slowly indicating potential processing challenges.

Solubility and Stability of Formulations with Varied Co-Solvent and CD Content

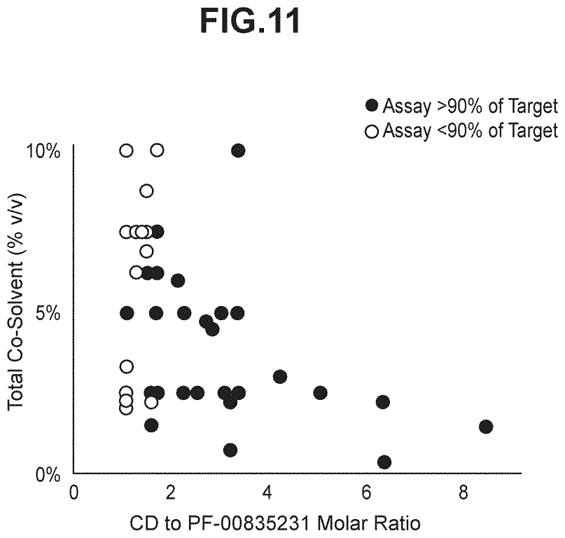

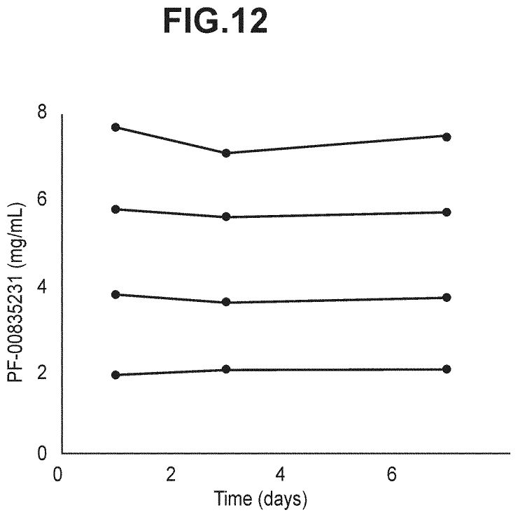

To further understand the impact of co-solvent and CD content on PF-00835231 solubility and physical stability, solutions with varied concentrations were prepared as described above. PF-00835231 was solubilized in a fixed volume of co-solvent at a fixed volume fraction of 75% PEG400 to 25% ethanol. The solutions were subsequently mixed with aqueous solutions to produce pharmaceutical compositions with approximately 5 mM citrate buffer and varied concentrations of SBE-β-CD or HP-β-CD. All solutions were then mixed for approximately 48 hours to enable full equilibration at room temperature and the solutions were subsequently filtered, as described above, and analyzed for PF-00835231 assay via HPLC against a PF-00835231 standard. This data is reported in FIG. 11, based on whether the formulations were within 90% of the assay target.

Based on the data depicted in FIG. 11, greater physical stability is observed for formulations with higher CD to PF-00835321 molar ratios and lower total co-solvent content. More CD relative to PF-00835231 likely helps to favor complexation and thus solubilization, while more co-solvent disrupts complexation and inhibits solubilization.

### Formulation Example F10: Solubility and Stability of Formulations with 80 mg/mL SBE-β-CD, Varied Co-Solvent Levels of PEG400 and Ethanol, and Varied PF-00835231 Concentration

To investigate whether stable solutions can be prepared at higher concentrations of PF-00835231, solutions were prepared with higher CD concentrations than Formulation Example F7. PF-00835231 was solubilized in a fixed volume of co-solvent at a fixed volume fraction of approximately 3:1 PEG400:ethanol. The solutions were subsequently mixed with aqueous solutions to final concentrations of approximately 80 mg/mL SBE-β-CD and 5 mM citrate buffer. These dilutions resulted in total co-solvent concentrations of approximately 1.5%, 3.0%, 4.5%, and 6.0% v/v. The target compositions have a CD:PF-0083231 molar ratio of approximately 8.4:1, 4.2:1, 2.8:1, and 2.1:1 for PF-00835231 concentrations of 2 mg/mL, 4 mg/mL, 6 mg/mL, and 8 mg/mL, respectively. The highest concentration formulation would enable delivery of up to 2 g, 4 g, or 8 g dose of PF-00835231 in a 250 mL, 500 mL, or 1000 mL administration volume, although the higher administration volumes may be limited by precedented levels of excipients.

Specifically, a 10 mL stock solution of co-solvent was prepared in a 10 mL volumetric flasks through measuring 2.5 mL of ethanol (Pharmco-AAPER, ACS/USP Grade) followed by dilution to volume with PEG400 (Fisher Chemical, Carbowax, NF Grade) to prepare stock solutions of approximately 75% PEG400/25% ethanol by volume. Flask was inverted several times to mix and placed in 50° C. oven for approximately 30 minutes prior to experiment. An approximately 300 mg/mL SBE-D-CD stock solution was prepared by weighing approximately 3.00 g of SBE-β-CD (Carbosynth, Pharma Grade) powder in a 10 mL volumetric flask and diluting to volume with purified water. The flask was capped and inverted to mix until clear.

A PF-00835231 stock solution was prepared in a 2 mL HPLC vials by first adding approximately 40 mg of the hydrate form of PF-00835231 to the vial, followed by addition of approximately 300 μL of the heated co-solvent stock mixture. The solution was then vortexed to mix for ˜1 minute. The resultant PF-00835231 stock solution was placed in a 50° C. oven and removed every 5 minutes to vortex until fully dissolved.

In separate 2 mL HPLC vials, 100 μL of an approximately 50 mM citrate solution adjusted to pH 5.0 and 267 μL of 300 mg/mL SBE-β-CD stock solution were mixed. Subsequently, 618 μL, 603 μL, 588 μL, or 573 μL of purified water was added to the vials corresponding to 2, 4, 6, or 8 mg/mL PF-00835231. After this addition, 15, 30, 45, or 60 mL of PF-00835231 solution was then transferred to the CD-containing solutions to create 1 mL of approximately 2 mg/mL, 4 mg/mL, 6 mg/mL, or 8 mg/mL PF-00835231 solutions respectively. The solutions were then capped and vortexed to mix. Solutions were then placed on a shaker at ambient conditions.

An aliquot of each formulation was removed after approximately 1, 3, and 7 days for assay determination via HPLC. Specifically, 150 μL aliquots were added to a centrifugal filter with a 0.1 μm PVDF filter and centrifuged for 3 minutes at 13,000 rcf. The filtrate was collected and analyzed via HPLC against a PF-00835231 standard to provide an assay value for PF-00835231, which is reported in FIG. 12. FIG. 12 depicts assay values of filtered PF-00835231 formulations containing ethanol, PEG400, and SBE-β-CD over 7 days at 4 different PF-00835231 concentrations of 2 mg/mL, 4 mg/mL, 6 mg/mL and 8 mg/mL. The assay values do not change over 7 days, which reflects the physical stability of the formulations. Potency was not corrected for impurities and water content, which led to assay values below target.

When the solubility data in FIG. 12 (up to 8 mg/mL of PF-00835231) is compared to the target infusion concentration range for PF-00835231 of 0.2 to 13.2 mg/mL, this data shows that SBE-β-CD at approximately 80 mg/mL with ethanol and PEG400 could cover almost the full target concentration range. If prepared in the target infusion volumes, these pharmaceutical compositions would contain excipients are within the precedented daily dose levels for IV administered drug products.

### Formulation Example F11: Scale-Up, Chemical Stability, and Physical Stability of Formulations with 80 mg/mL SBE-β-CD, 1.1% v/v ethanol, 3.4% v/v PEG400, and 6 mg/mL PF-00835231

### Formulation Preparation

To further assess the robustness of formulations prepared in Formulation Example F10, a single formulation was selected to scale-up for use in a stability study. Specifically, the formulation with 80 mg/mL SBE-β-CD, 4.5% v/v total co-solvent (1.1% v/v ethanol, 3.4% v/v PEG400), and 6 mg/mL PF-00835231 was prepared. The target composition has a CD:PF-00835231 molar ratio of approximately 2.8. This formulation would enable delivery of a 1.5 g, 3 g, or 6 g dose of PF-00835231 in a 250 mL, 500 mL, or 1000 mL administration volume, although the higher administration volumes may be limited by precedented levels of excipients.

To prepare this formulation, a 10 mL stock solution of co-solvent was prepared in a 10 mL volumetric flask through measuring 2.5 mL of ethanol (Pharmco-AAPER, ACS/USP Grade) followed by dilution to volume with PEG400 (Fisher Chemical, Carbowax, NF Grade) to prepare stock solutions of approximately 75% PEG400/25% ethanol by volume. The flask was inverted several times to mix and placed in a 50° C. oven for 30 approximately minutes prior to experiment. An approximately 300 mg/mL SBE-β-CD stock solution was prepared by weighing approximately 3.00 g of SBE-β-CD powder in a 10 mL volumetric flask and diluting to volume with purified water. The flask was capped and inverted to mix until clear. An additional 20 mL solution of 300 mg/mL SBE-β-CD was similarly prepared.

In a 100 mL volumetric flask, 10.5 mL of an approximately 50 mM citrate solution adjusted to pH 5 and 28 mL of 300 mg/mL SBE-β-CD stock solution were mixed, followed by dilution to the target volume with purified water. The flask was capped and inverted to mix. This results in an aqueous solution that will prepare a formulation with a final concentration of 5 mM citrate buffer and 80 mg/mL SBE-β-CD.

A PF-00835231 stock solution was prepared in a 2 mL HPLC vials by first adding approximately 200 mg of the hydrate form of PF-00835231 to the vial, followed by addition of approximately 1.5 mL of the heated co-solvent stock mixture. The solution was then vortexed to mix for ˜1 minute. The resultant PF-00835231 stock solution was placed in a 50° C. oven and removed every 5 minutes to vortex until fully dissolved.

14.29 mL of the citrate buffer and SBE-β-CD stock solution was then added to two separate 20 mL scintillation vials. After this addition, 0.71 mL of PF-00835231 stock solution was then transferred to the vials to create 15 mL of approximately 6 mg/mL PF-00835231. The solutions were then capped and vortexed to mix. The drug product solutions were mixed overnight and filtered through a 0.2 um PVDF filter. 0.5 mL aliquots of drug product solutions were then filled into 4 mL vials, stoppered, crimped, and placed in temperature-controlled chambers at −20° C., 4° C., and 25° C.

### Physical Stability

An aliquot of each formulation was removed after either 1, 3, 7, 16, or 30 days for assay and purity determination via HPLC. Specifically, 0.2 mL aliquots were added to a centrifugal filter with a 0.1 μm PVDF filter and centrifuged for 3 minutes at 13,000 rcf. The filtrate was collected and analyzed via HPLC against a PF-00835231 standard to provide an assay value for PF-00835231.

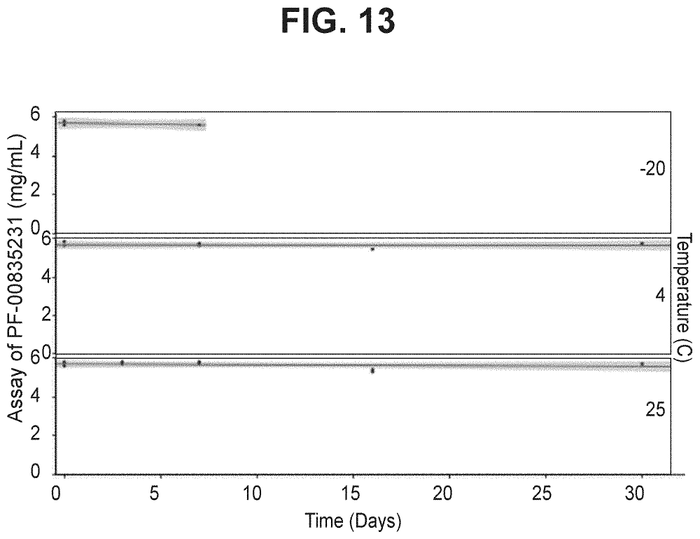

If the prepared drug product solutions were supersaturated or otherwise not physically stable, a decrease in the assay value over time would be expected, as the filtration step during HPLC sample preparation should remove any precipitated PF-00835231. Furthermore, if the solutions were supersaturated at room temperature, exposure to refrigerated or frozen storage temperatures should induce precipitation and cause a decrease in assay. Assay values at −20° C. appear consistent over the 7 day period tested, and assay values at 4° C. and 25° C. appear consistent over the 30 day period tested, as shown in Figure. Consequently, this data suggests that the solutions are physically stable over the investigated time period. FIG. 13 depicts PF-00835231 assay values in mg/mL are plotted as a function of time in days for 3 temperatures: −20° C. (top), 4° C. (center), and 25° C. (bottom). For each condition, data from two separate samples is plotted and is shown with a linear fit to the data. The data shows consistent assay values for all samples over the investigated time period.

### Admixture Physical Stability

To further assess physical stability, drug product solutions were diluted in 0.9% w/v sodium chloride by a factor of 2.5× and 10× to concentrations of 2.4 mg/mL and 0.6 mg/mL, respectively. These dilutions mimic possible IV administration conditions, where the drug product may be prepared as a ready-to-dilute concentrate that is diluted prior to administration. Dilution experiments also further assess the physical and chemical stability of the formulation.

To prepare the 2.5× dilution, 1.6 mL of the filtered formulation was added to 2.4 mL of 0.9% w/v sodium chloride in a 4 mL vial. To prepare the 10× dilution, 0.4 mL of the filtered formulation was added to 3.6 mL of 0.9% w/v sodium chloride in a 4 mL vial. An aliquot of each formulation was removed after 0 and 3 days at 25° C. for assay and purity determination via HPLC. Specifically, 0.2 mL aliquots were added to a centrifugal filter with a 0.1 μm PVDF filter and centrifuged for 3 minutes at 13,000 rcf. The filtrate was collected and analyzed via HPLC against a PF-00835231 standard to provide an assay value for PF-00835231.

When samples with no dilution, 2.5× dilution, and 10× dilution are compared, assay values are consistent over 3 days, indicative of physical stability, as shown in Formulation Table F12. In addition, the chemical stability of PF-00835231 is comparable across each dilution.

FORMULATION TABLE F12
PF-00835231 assay and purity with no dilution, 2.5x dilution,
and 10x dilution are shown after 0 days and 3 days at 25° C.

| | Assay | Assay | Total Impurities | Total Impurities |
|---|---|---|---|---|
| Dilution | (mg/mL) - | (mg/mL) - | (% peak area) - | (% peak area) - |
| Factor | 0 days | 3 days | 0 days | 3 days |
| No | 5.8 | 5.8 | 1.25 | 1.44 |
| dilution | 5.6 | 5.7 | 1.25 | 1.48 |
| 2.5x | 2.3 | 2.3 | 1.23 | 1.38 |
| | 2.2 | 2.2 | 1.20 | 1.37 |
| 10x | 0.6 | 0.6 | 1.20 | 1.33 |
| | 0.6 | 0.6 | 1.20 | 1.33 |

### Formulation Example F12: Chemical Stability of PF-00835231 Formulations with and without CD

To investigate the impact of CD on the chemical stability of PF-00835231, solutions were prepared with and without CD. Specifically, 10 mL solutions were prepared with a final composition of approximately 1 mg/mL PF-00835231, 5% v/v total co-solvent (2.5% v/v PEG400, 2.5% v/v ethanol), 5 mM citrate buffer, and optionally 15 mg/mL SBE-β-CD. Two formulations were prepared at pH 4 and 2 formulations were prepared at pH 5 for solutions with and without CD to understand the impact of pH. For formulations with CDs, the target composition has a CD:PF-00835231 molar ratio of approximately 3.2. This formulation would enable delivery of a 0.25 g, 0.5 g, or 1 g dose of PF-00835231 in a 250 mL, 500 mL, or 1000 mL administration volume.

Specifically, an approximately 75 mg/mL SBE-β-CD stock solution was prepared by weighing approximately 3.75 g of SBE-β-CD powder in a 50 mL volumetric flask and diluting to volume with purified water. The flask was capped and inverted several times to mix until the solution was clear. 20 mL of the approximately 75 mg/mL SBE-β-CD stock solution was subsequently mixed with 10 mL of approximately 50 mM buffer at either pH 4 or 5 in a 100 mL volumetric flask. The resultant solutions were then diluted to the target volume with purified water and inverted to mix until clear. The resultant solutions possessed a final composition of approximately 15 mg/mL SBE-β-CD and 5 mM of citrate buffer at either pH 4 or 5. 5 mM citrate buffers at pH 4 and 5 were similarly prepared without SBE-3-CD.

To 2 mL HPLC vials, approximately 10 mg of the hydrate form of PF-00835231 was added, followed by 250 mL of PEG400 (Fisher Chemical, Carbowax, NF Grade) and 250 mL of ethanol (Pharmco-AAPER, ACS/USP Grade). The PF-00835231 formulation was sonicated until dissolved.

To 10 mL scintillation vials, 9.5 mL of an approximately 15 mg/mL SBE-j-CD stock solution or 9.5 mL of citrate buffer was added, followed by the PF-00835231 dissolved in co-solvent. The formulations were then sub-divided and placed on stability at 4° C., 22° C., or 40° C. Aliquots were removed after approximately 0, 1, 3, and 7 days and the purity profile was examined via HPLC.

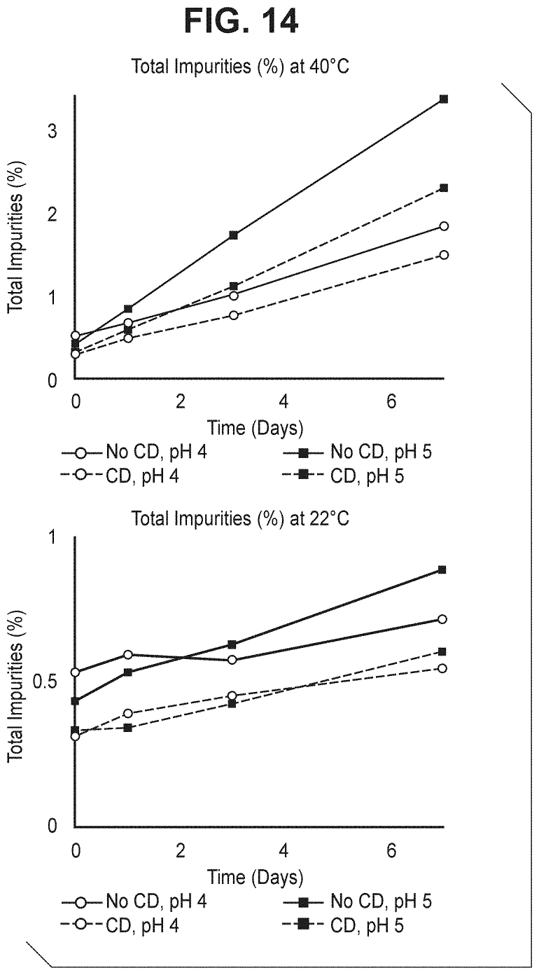

From this experiment, no degradation was observed at 4° C. for all samples. The samples with CDs were physically stable to multiple rounds of freezing and thawing (i.e. no particulate formed), while samples without CDs showed precipitation after thawing. At 22° C., less than 0.5% decrease in chromatographic purity was observed for all samples, with the lowest degradation at pH 4 and in the presence of SBE-β-CD. The same trends are observed at 40° C. Consequently, this experiment surprisingly demonstrates that CD provides a protective effect for both the chemical and physical stability of PF-00835231. FIG. 14 depicts the chemical stability of PF-00835231 in solutions with CD (dashed) and without CD (solid) at pH 4 (black, open circles) and pH 5 (gray, closed circles) at 40° C. (top) and 22° C. (bottom).

### Reference Example 8: N-((1S)-1-{[((1S)-3-hydroxy-2-oxo-1-{[3S)-2-oxopyrrolidin-3-yl]methyl}propyl)amino]carbonyl}pentyl)-4-methoxy-1H-indole-2-carboxamide

Following the procedure described for the preparation of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide but substituting N-((1S)-1-{[((1S)-3-chloro-2-oxo-1-{[(3S)-2-oxopyrrolidin-3-yl]methyl}propyl)amino]carbonyl}pentyl)-4-methoxy-1H-indole-2-carboxamide and making non critical variations provided a crude yellow foam. This material was purified by Biotage MPLC (25M column, 5-6% methanol/chloroform) to afford 82 mg (35%) of the title compound as an off-white solid. ¹H NMR (DMSO-d₆) δ 11.57 (s, 1H), 8.42 (d, J=8 Hz, 1H), 8.38 (d, J=4 Hz, 1H), 7.62 (s, 1H), 7.35 (s, 1H), 7.09 (t, J=8 Hz, 1H), 6.99 (d, J=8 Hz, 1H), 6.49 (d, J=8 Hz, 1H), 5.06 (t, J=8 Hz, 1H), 4.44 (m, 2H), 4.25 (dd, J=8, 20 Hz, 1H), 4.14 (dd, J=8, 20 Hz, 1H), 3.87 (s, 3H), 3.08 (m, 2H), 2.28 (m, 1H), 2.10 (m, 1H), 1.91 (m, 1H), 1.74-1.54 (m, 4H), 1.32 (m, 4H), 0.87 (t, J=8 Hz, 3H); MS (ESI+) for C₂₄H₃₂N₄O₈ m/z 473.2 (M+H)⁺; Anal. Calcd from C₂₄H₃₂N₄O₆ •0.6 H₂O & •0.2 ethyl acetate: C, 59.46; H, 7.01; N, 11.18. Found: C, 53.37; H, 6.94; N, 11.23: HRMS (ESI+) Calcd C₂₄H₃₂N₄O₆+H1 473.2395, found 473.2382.

### Preparation of Intermediate: methyl N-[9H-fluoren-9-ylmethoxy)carbonyl]-5-methyl-L-norleucinate

To a solution of N-[(9H-fluoren-9-ylmethoxy)carbonyl]-5-methyl-L-norleucine (2.14 g, 5.8 mmol) in methanol (15 mL) is added toluene (30 mL) followed by dropwise addition of TMS-diazomethane (2.9 mL, 2M in Hexane, 5.8 mmol). TLC analysis indicated incomplete reaction and TMS-diazomethane was added drop-wise until a yellow color persisted. At this time, the reaction was quenched by the addition of AcOH (1 mL) followed by concentration in vacuo. The residue was purified by Biotage flash chromatography, eluting with ethyl acetate/Hexane to afford the title compound as a white solid, 2.18 g, 98%. ¹H NMR (400 MHz, CDCl₃) δ 7.76 (2H, d, J=7.6 Hz), 7.60 (2H, dd, J=7.2, 3.9 Hz), 7.40 (2H, t, J=7.2 Hz), 7.31 (2H, t, J=7.5 Hz), 5.26 (1H, d J=8.6 Hz), 4.31-4.51 (3H, m), 4.23 (1H, t, J=7.1. Hz), 3.75 (3H, s), 1.78-1.93 (1H, m), 1.6-1.76 (1H, m), 1.45-1.60 (1H, m), 1.05-1.34 (2H, m), 0.88 (d, J=4 Hz, 3H), 0.86 (d, J=4 Hz, 3H), MS (APCI+) for C₂₃H₂₇NO₄ m/z 160.1 (M-Fmoc+H)⁺.

### Preparation of Intermediate: methyl N-(tert-butoxycarbonyl)-5-methyl-L-norleucinate

To a solution of N-[(9H-fluoren-9-ylmethoxy)carbonyl]-5-methyl-L-norleucine (2.18 g, 5.72 mmol) in DMF (50 mL) was added KF (2.33 g, 40.04 mmol) followed by triethylamine (1.70 mL, 12.24 mmol) and di-tert-butyl decarbonate (7.39 mmol) and the mixture stirred at ambient temperature. After 4 hours, TLC analysis indicated incomplete reaction and the reaction mixture was treated with a section portion of KF (2.7 g, 46.55 mmol) and BOC₂O (800 mg, 3.67 mmol). After 16 hours, the mixture was diluted with diethyl ether (300 mL), washed with satd. NaHCO₃ (2×50 mL), 1M hydrochloric acid (2×50 mL), NaHCO₃ (50 mL), brine (50 mL), dried over MgSO₄, filtered and the solvents evaporated in vacuo to yield the crude product, which was purified by Biotage flash chromatography eluting with dichloromethane/hexane to afford the title compound as a clear oil, 980 mg, 66%. ¹H NMR (400 MHz, CDCl₃) δ 4.96 (1H, d, J=6.8 Hz), 4.21-4.32 (1H, m), 3.72 (3H, s) 1.72-1.85 (1H, m), 1.46-166 (2H, m) 1.43 (9H, s), 1.11-1.29 (2H, m) 0.99 (d, J=4 Hz, 3H), 0.86, (d, J=4 Hz, 3H); MS (API-ES+) for C₁₃H₂₅NO₄ m/z 282.2 (M+Na)⁺.

### Preparation of Intermediate: N-(tert-butoxycarbonyl)-5-methyl-L-norleucinate

To a solution of N-(tert-butoxycarbonyl)-5-methyl-L-norleucinate (980 mg, 3.78 mmol) in THF (30 mL) at 0° C. was added a solution (pre-cooled to 5° C.) of LiOH (1M, 11.3 mL, 11.33 mmol) and the resulting mixture stirred at 0° C. for 1 hour, then allowed to warm to ambient temperature. The reaction was acidified to pH 2 with 1M hydrochloric acid and extracted with ethyl acetate (3×60 mL). The combined organics were washed with brine (100 mL) dried over MgSO₄, filtered and the solvent removed in vacuo to yield the title compound as a clear oil, 990 mg, 99%. ¹H NMR (400 MHz, CDCl₃) δ 4.96 (1H, d, J=7.8 Hz), 4.23-4.34 (1H, m), 1.75-1.93 (2H, m), 1.60-1.72 (1H, m), 1.50-1.59 (1H, m), 1.44 (9H, s), 1.19-1.30 (1H, m), 0.88 (d, J=4 Hz, 3H), 0.86 (d, J=4 Hz, 3H); MS (API-ES+) for C₁₂H₂₃NO₄ m/z 268.1 (M+Na)⁺.

### Preparation of Intermediate: N²-(tert-butoxycarbonyl)-N¹-((1S)-3-chloro-2-oxo-1-{[(3S)-2-oxopyrrolidin-3-yl]methyl}propyl)-5-methyl-L-norleucinamide

Following the procedure described for the preparation of N²-(tert-butoxycarbonyl)-N¹-((1S)-3-chloro-2-oxo-1-{[(3S)-2-oxopyrrolidin-3-yl]methyl}propyl-L-leucinamide but substituted N-(tert-butoxycarbonyl)-5-methyl-L-norleucine and making non-critical variations provided a crude golden oil. This material was purified by Biotage flash chromatography, eluting with methanol/dichloromethane to afford the title compound as an off-white solid, 360 mg, 41%. ¹H NMR (400 MHz, DMSO-d₆) δ 8.45 (1H, d, J=8.1 Hz), 7.62 (1H, s), 7.02 (1H, d, J=7.1 Hz), 4.49-4.62 (2H, m), 4.33-4.44 (1H, m), 3.78 (1H, m), 3.15 (1H, t, J=8.7 Hz), 3.00-3.10 (1H, M), 2.18-2.30 (1H, m), 2.04-2.14 (1H, m), 1.92-2.02 (1H, M), 1.40-1.68 (5H, m), 1.36 (9H, s), 1.05-1.25 (2H, m, J=7.3 Hz), 0.83 (3H, d, J=1.52 Hz), 0.82 (3H, d, J=1.52 Hz); MS (API-ES+) for C₂₀H₃₄N₃O₅Cl m/z 454.2 (M+Na)⁺.

### Reference Example 16: N-((1S)-1-{(((1S)-3-hydroxy-2-oxo-1-{[(3S)-2-oxopyrrolidin-3-yl]methyl}propyl)amino]carbonyl}-3,3-dimethylbutyl)-1H-indole-2-carboxamide

Following the procedure described for the preparation of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide but substituting N-((1S)-1-{[((1S)-3-chloro-2-oxo-1-{[(3S)-2-oxopyrrolidin-3-yl]methyl}propyl)amino]carbonyl}-3,3-dimethylbutyl)-1H-indole-2-carboxamide and making non critical variations provided a crude yellow foam. This material was purified by Biotage MPLC (40M column, 4.5-5.5% methanol/chloroform) to afford 730 mg (72%) of the title compound as a white solid. ¹H NMR (DMSO-d₆) δ 11.59 (s, 1H), 8.49 (d, J=8 Hz, 1H), 8.43 (d, J=8 Hz, 1H), 7.62 (s, 1H), 7.60 (s, 1H), 7.41 (d, J=8 Hz, 1H), 7.23 (s, 1H), 7.17 (t, J=8 Hz, 1H), 7.02 (t, J=8 Hz, 1H), 5.05 (t, J=8 Hz, 1H), 4.56 (m, 1H), 4.43 (m, 1H), 4.25 (dd, J=8, 20 Hz, 1H), 4.13 (dd, J=8, 20 Hz, 1H), 3.10 (m, 2H), 2.25 (m, 1H), 2.07 (m, 1H), 1.93 (m, 1H), 1.80 (m, 1H), 1.64 (m, 3H), 0.94 (s, 9H); MS (ESI+) for C₂₄H₃₂N₄O₅ m/z 457.1 (M+H)⁺; Anal. Calcd for C₂₄H₃₂N₄O₅•0.2 CHCl₃•0.2 ethyl acetate•0.25 H₂O: C, 59.75; H, 6.88; N, 11.15. Found C, 59.67; H, 6.72; N, 11.03; HRMS (ESI+) Calcd for C₂₄H₃₂N₄O₅ 457.2446, found 457.2439.

### Reference Example 23: N-((1S)-3-hydroxy-2-oxo-1-{[(3S)-2-oxopyrrolidin-3-yl]methyl}propyl)-N-[(4-methoxy-1H-indol-2-yl)carbonyl]-L-phenylalaninamide

Following the procedure described for the preparation of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide but substituting N-((1S)-3-chloro-2-oxo-1-{[(3S)-2-oxopyrrolidin-3-yl]methyl}propyl)-N-[(4-methoxy-1H-indol-2-yl)carbonyl]-L-phenylalaninamide and making non-critical variations provided a crude greenish gum. The material was purified by Biotage flash chromatography, eluting with methanol/dichloromethane to afford the title compound as an off-white solid, 123 mg, 44%. ¹H NMR (400 MHz DMSO-d₆) δ 11.50 (1H, d, J=2.0 Hz), 8.58 (2H, dd, J=8.2, 3.9 Hz), 7.63 (1H, s), 7.35-7.43 (2H, m), 7.31 (1H, d, J=1.8 Hz), 7.27 (2H, t, J=7.6 Hz), 7.17 (1H, t, J=7.3 Hz), 7.08 (1H, t, J=8.0 Hz), 6.98 (1H, d, J=8.1 Hz), 6.48 (1H, d, J=7.8 Hz), 5.06 (1H, d, J=6.1 Hz), 4.72 (1H, m), 4.48 (1H, m), 4.16 (2H, m), 3.89 (3H, s), 2.97-3.18 (4H, m), 2.24-2.36 (1H, m), 2.04-2.18 (1H, m), 1.88-2.01 (1H, m), 1.55-1.76 (2H, m); MS (APCI+) for C₂₇H₃₀N₄O₆ m/z 507.1 (M+H)⁺.

### Preparation of Intermediate: N²-(tert-butoxycarbonyl)-N¹-((1S)-3-chloro-2-oxo-1-{[(3S)-2-oxopyrrolidin-3-yl]methyl}propyl)-N²-methyl-L-leucinamide

Following the procedure described for the preparation of N²-(tert-butoxycarbonyl)-N¹-((1S)-3-chloro-2-oxo-1-{[(3S)-2-oxopyrrolidin-3-yl]methyl}propyl)-L-leucinamide but substituting Boc-N-methyl-Leu-OH and making non-critical variations provided a crude green gum. This material was purified by Biotage flash chromatography, eluting with methanol/dichloromethane to afford the title compound as an orange foam, 2.08 g, 46%. ¹H NMR (400 MHz DMSO-d₆) δ 8.54 (1H, d, J=7.1 Hz), 7.69 (1H, d, J=11.1 Hz), 4.58 (2H, s), 4.48 (1H, d, J=11.9 Hz), 4.40 (1H, s), 3.02-3.22 (2H, m), 2.69-2.79 (3H, m), 2.14-2.27 (1H, m), 2.03-2.14 (1H, m), 1.87-2.00 (1H, m), 1.48-1.76 (4H, m), 1.38 (9H, brd, J=5.8 Hz), 0.79-0.97 (6H, m).

### Reference Example 36: (3S)-3-({N-[(4-methoxy-1H-indol-2-yl)carbonyl]-L-leucyl}amino)-2-oxo-4-[(3S)-2-oxopyrrolidin-3-yl]butyl acetate

An oven-dried 40 mL scintillation vial with spinbar was charged with acetic acid (27 mg, 0.46 mmol) followed by a solution of N-((1S)-1-{[((1S)-3-chloro-2-oxo-1-{[(3S)-2-oxopyrrolidin-3-yl]methyl}propyl)amino]carbonyl}-3-methylbutyl)-4-methoxy-1H-indole-2-carboxamide (172 mg, 0.35 mmol) in DMF (3.5 mL), and purged with N₂. This pale yellow solution was then treated with CsF (122 mg, 0.81 mmol), sealed with a Teflon-lined screwcap, and heated at 65° C. on a reaction block with vigorous stirring. After 3 hours, the reaction was cooled to RT, diluted with water (30 mL), and extracted with dichloromethane (4×7 mL). The combined organic layers were washed with water (2×20 mL), brine (20 mL), and concentrated in vacuo. This material was purified by Biotage MPLC (25M, 2.5-4.5% methanol/dichloromethane) to afford 78 mg (45%) of the title compound as a white solid. ¹H NMR (400 MHz, DMSO-d₆) δ 11.57 (s, 1H), 8.57 (d, J=7.8 Hz, 1H), 8.43 (d, J=7.6 Hz, 1H), 7.64 (s, 1H), 7.36 (d, J=1.8 Hz, 1H), 7.08 (t, J=8.0 Hz, 1H), 6.99 (d, J=8.1 Hz, 1H), 6.49 (d, J=7.6 Hz, 1H), 4.83 (d, J=3.0 Hz, 1H), 4.76-4.95 (m, 1H), 4.35-4.50 (m, 2H), 3.87 (s, 3H), 3.03-3.17 (m, 2H), 2.22-2.35 (m, 1H), 2.09-2.22 (m, 1H), 2.07 (s, 3H), 1.90-2.04 (m, 1H), 1.65-1.77 (m, 2H), 1.48-1.65 (m, 3H), 0.94 (d, J=6.3 Hz, 3H), 0.89 (d, J=6.3 Hz, 3H); MS (ESI+) for C₂₆H₃₄N₄O₇ m/z 515.2 (M+H)⁺.

### Reference Example 37: (3S)-3-({N-[(4-methoxy-1H-indol-2-yl)carbonyl]-L-leucyl}amino)-2-oxo-4-[(3S)-2-oxopyrrolidin-3-yl]butyl cyclopropanecarboxylate

Following the procedure described for the preparation of (3S)-3-({N-[(4-methoxy-1H-indol-2-yl)carbonyl]-L-leucyl}amino)-2-oxo-4-[(3S)-2-oxopyrrolidin-3-yl]butyl acetate but substituting cyclopropanecarboxylic acid and making non-critical variations provided a crude product. This material was purified by Biotage MPLC (25M, 2.5-4.5% methanol/dichloromethane) to afford 82 mg (43%) of the title compound. ¹H NMR (400 MHz, DMSO-d₆) δ 11.57 (d, J=2.0 Hz, 1H), 8.56 (d, J=7.8 Hz, 1H), 8.43 (d, J=7.6 Hz, 1H), 7.63 (s, 1H), 7.36 (d, J=1.5 Hz, 1H), 7.08 (t, J=8.0 Hz, 1H), 6.97-7.02 (m, 1H), 6.49 (d, J=7.6 Hz, 1H), 4.85 (d, 1H), 4.78-4.96 (m, 1H), 4.33-4.51 (m, 2H), 3.87 (s, 3H), 3.02-3.16 (m, 2H), 2.22-2.35 (m, 1H), 2.01-2.11 (m, 1H), 1.89-2.00 (m, 1H), 1.65-1.77 (m, 3H), 1.46-1.65 (m, 3H), 0.81-0.98 (m, 10H); MS (ESI+) for C₂₈H₃₆N₄O₇ m/z 541.2 (M+H)⁺.

### Reference Example 39: (3S)-3-({4-methyl-N-[(2R)-tetrahydrofuran-2-ylcarbonyl]-L-leucyl}amino)-2-oxo-4-[(3S)-2-oxopyrrolidin-3-yl]butyl 2,6-dichlorobenzoate

Following the procedure described for the preparation of (3S)-3-({N-[(4-methoxy-1H-indol-2-yl)carbonyl]-L-leucyl}amino)-2-oxo-4-[(3S)-2-oxopyrrolidin-3-yl]butyl acetate but substituting N¹-((1S)-3-chloro-2-oxo-1-{[(3S)-2-oxopyrrolidin-3-yl]methyl}propyl)-4-methyl-N²-[(2R)-tetrahydrofuran-2-ylcarbonyl]-L-leucinamide and 2,6-dichlorobenzoic acid and making non-critical variations provided a light amber residue. The residue was purified by preparative HPLC (Luna 10μ C18) eluting with a gradient of MeCN containing 0.1% AcOH in water containing 0.1% AcOH to give 0.155 g (54%) of the title compound as a cream colored solid. ¹H NMR (300 MHz, DMSO-d₆) δ 8.52 (d, J=8 Hz, 1H), 7.79 (d, J=8 Hz, 1H), 7.71 (s, 1H), 7.66-7.55 (m, 3H), 5.18 (s, 2H), 4.54-4.40 (m, 1H), 4.37-4.35 (m, 1H), 4.25 (m, 1H), 3.98-3.91 (m, 1H), 3.84-3.72 (m, 1H), 3.23-3.07 (m, 2H), 2.33-2.23 (m, 1H), 2.16-2.05 (m, 2H), 1.86-1.74 (m, 3H), 1.72-1.62 (m, 5H), 0.89 (s, 9H); MS (ESI+) for C₂₇H₃₅Cl₂N₃O₇ m/z 584 (M+H). Anal. Calcd for C₂₇H₃₅Cl₂N₃O₇•0.5 H₂O: C, 54.64; H, 6.11; N, 7.08. Found: C, 54.26; H, 6.00; N, 6.87. HRMS (ESI+) Calcd for C₂₇H₃₅Cl₂N₃O₇+H1 584.1925, found 584.1921.

The compounds N—((S)-1-(((S)-1-(benzo[d]thiazol-2-yl)-1-oxo-3-((S)-2-oxopyrrolidin-3-yl)propan-2-yl)amino)-3-cyclopropyl-1-oxopropan-2-yl)picolinamide; N—((S)-1-(((S)-1-(benzo[d]thiazol-2-yl)-1-oxo-3-((S)-2-oxopyrrolidin-3-yl)propan-2-yl)amino)-3-cyclopentyl-1-oxopropan-2-yl)-4-methoxy-1H-indole-2-carboxamide; and N—((S)-2-(((S)-1-(benzo[d]thiazol-2-yl)-1-oxo-3-((S)-2-oxopyrrolidin-3-yl)propan-2-yl)amino)-1-cyclopentyl-2-oxoethyl)-4-methoxy-1H-indole-2-carboxamide; were prepared in an analogous manner to those set forth above and other compounds described in WO2005/113580 and as described below.

### Preparation of N-(tert-butoxycarbonyl)-3-[(3S)-2-oxopyrrolidin-3-yl]-L-alanine

To a solution of methyl N-(tert-butoxycarbonyl)-3-[(3S)-2-oxopyrrolisin-3-yl]-L-alaninate (prepared according to PCT International Appl. Publication WO 01/14329 A1, Compound 3, Scheme 9 therein) (20 g, 70 mmol) in methanol (100 mL) cooled to 0° C. was slowly added a solution of NaOH (14 g, 350 mmol) in water (120 mL). The mixture was stirred at 0° C. for 1 h and concentrated in vacuo keeping the temperature below 25° C. until the sodium salt of the product precipitated. The mixture was neutralized with conc. aqueous HCl solution to pH 5 with ice bath cooling and further acidified to pH 1 with 1N HCl aqueous solution. The mixture was extracted with ethyl acetate 3 times. The combined organic phase was washed with brine, dried over MgSO₄, filtered, concentrated in vacuo and further dried under high vacuum overnight to give the desired acid (19 g, 100% yield). ¹H NMR (400 MHz, CD₃OD) δ 4.12 (m, 1H), 3.33 (m, 2H), 2.48 (m, 1H), 2.35 (m, 1H), 2.08 (m, 1H), 1.81 (m, 2H), 1.44 (s, 9H).

### Preparation of tert-butyl ((1S)-2-[methoxy(methyl)amino]-2-oxo-1-{[(3S)-2-oxopyrrolidin-3-yl]methyl}ethyl)carbamate

To a solution of N-(tert-butoxycarbonyl)-3-[(3S)-2-oxopyrrolidin-3-yl]-L-alanine (19 g, 70 mmol) in dichloromethane (150 mL) was added N,O-dimethylhydroxylamine hydrochloride (6.97 g, 70 mmol), N-methylmorpholine (26.9 mL, 24.78 g, 245 mmol) and hydroxybenzotriazole hydrate (9.46 g, 70 mmol). The mixture was cooled to 0° C., and 1-(3-dimethylaminopropyl)-3-ethylcarbodiimide hydrochloride (16.1 g, 84 mmol) was added as a solid. The mixture was stirred at 0° C. for 4 h, and to this was added 150 mL 1N HCl aqueous solution. The organic phase was separated from the aqueous phase. The aqueous layer was extracted with dichloromethane (1×200 mL). The combined organic phase was dried over Na₂SO₄, filtered, and concentrated in vacuo. The residue was purified by flash column chromatography (eluting with 6-10% methanol in dichloromethane) to provide the desired product as a white solid (17 g, 77% yield). ¹H NMR (400 MHz, CDCl₃) δ 6.00 (bs, 1H), 5.41 (d, J=8.6 Hz, 1H), 4.68 (d, J=8.9 Hz, 1H), 3.78 (s, 3H), 3.35 (m, 2H), 3.21 (s, 3H), 2.50 (m, 2H), 2.11 (t, J=10.8 Hz), 1.84 (m, 1H), 1.68 (m, 1H), 1.43 (s, 9H). LCMS ESI (M+Na⁺): 338.1.

### Preparation of N-{(1S)-1-cyclopentyl-2-[((1S)-2-[methoxy(methyl)amino]-2-oxo-1-{[(3S)-2-oxopyrrolidin-3-yl]methyl}ethyl)amino]-2-oxoethyl}-4-methoxy-1H-indole carboxamide

To a solution of tert-butyl ((1S)-2-[methoxy(methyl)amino]-2-oxo-1-{[(3S)-2-oxopyrrolidin-3-yl]methyl}ethyl)carbamate (473 mg, 1.5 mmol) in anhydrous 1,4-dioxane is added 4.0 M HCl in 1,4-dioxane (˜10 equivalents). The mixture is stirred at room temperature overnight and concentrated in vacuo to yield the HCl salt of the deprotected amine as a white foam. The amine is dissolved in dichloromethane and N-methylmorpholine (˜4 equivalents), to this solution is added N-Boc-L-cyclopentylglycine (˜1 equivalent), hydroxybenzotriazole (˜1 equivalent) and 1-(3-dimethylaminopropyl)-3-ethylcarbodiimide hydrochloride (˜1.2 equivalents). The mixture is stirred at ambient temperature for 1 h and poured into 1N aqueous HCl. The organic phase is washed with 1N aqueous HCl, dried over Na₂SO₄, filtered and concentrated in vacuo. The residue is purified by flash chromatography to provide tert-butyl((1S)-1-cyclopentyl-2-(((2S)-1-(methoxy(methyl)amino)-1-oxo-3-(2-oxopyrrolidin-3-yl)propan-2-yl)amino)-2-oxoethyl)carbamate. The Boc protecting group of the tert-butyl((1S)-1-cyclopentyl-2-(((2S)-1-(methoxy(methyl) amino)-1-oxo-3-(2-oxopyrrolidin-3-yl)propan-2-yl)amino)-2-oxoethyl)carbamate is then removed by acid catalyzed deprotection (such as with HCl in dioxane) which following workup provides the amine, (2S)-2-((S)-2-amino-2-cyclopentylacetamido)-N-methoxy-N-methyl-3-(2-oxopyrrolidin-3-yl)propanamide. The (2S)-2-((S)-2-amino-2-cyclopentylacetamido)-N-methoxy-N-methyl-3-(2-oxopyrrolidin-3-yl)propanamide is then coupled with 4-methoxy-1H-indole-2-carboxylic acid in the presence of 1-(3-dimethylaminopropyl)-3-ethylcarbodiimide hydrochloride (˜1.2 equivalents), dimethylaminopyridine (DMAP) in N-methylmorpholine and dichloromethane. The desired compound, N-{(1S)-1-cyclopentyl-2-[((1S)-2-[methoxy(methyl)amino]-2-oxo-1-{[(3S)-2-oxopyrrolidin-3-yl]methyl}ethyl)amino]-2-oxoethyl}-4-methoxy-1H-indole carboxamide, was obtained as a white solid (699 mg, 90% yield). ¹H NMR (300 MHz, CDCl₃) δ 10.79 (s, 1H), 8.53 (bs, 1H), 7.15 (t, J=7.9 Hz, 1H), 7.09 (d, J=1.7 Hz, 1H), 7.04 (d, J=8.3 Hz, 1H), 6.94 (d, J=9 Hz, 1H), 6.48 (d, J=7.6 Hz, 1H), 6.39 (bs, 1H), 5.18-5.05 (m, 2H), 3.95 (s, 3H), 3.83 (s, 3H), 3.30 (s, 3H), 3.13 (m, 2H), 2.50-2.25 (m, 3H), 2.15 (m, 1H), 1.75-1.40 (m, 10H). LCMS ESI (M+H⁺) 514.2, (M+Na⁺) 536.2.

### Preparation of N-{(1S)-2-[((1S)-2-(1,3-benzothiazol-2-yl)-2-oxo-1-{[(3S)-2-oxopyrrolidin-3-yl]methyl}ethyl)amino]-1-cyclopentyl-2-oxoethyl}-4-methoxy-1H-indole-2-carboxamide

To a solution of benzothiazole (0.597 mL, 5.50 mmol) in anhydrous tetrahydrofuran at −78° C. is slowly added n-butyl lithium (2.5 M in hexanes, 2.20 mL, 5.50 mmol), the mixture is stirred at −78° C. for 30 minutes, to this is added a solution of N-{(1S)-1-cyclopentyl-2-[((1S)-2-[methoxy(methyl)amino]-2-oxo-1-{[(3S)-2-oxopyrrolidin-3-yl]methyl}ethyl)amino]-2-oxoethyl}-4-methoxy-1H-indole carboxamide (257 mg, 0.50 mmol) in anhydrous tetrahydrofuran. The resulting mixture is stirred at −78° C. for 2 h and is quenched with saturated aqueous ammonium chloride. The mixture is allowed to warm to room temperature then poured into ethyl acetate and water. The organic layer is separated, washed with water then brine, dried over Na₂SO₄, filtered and concentrated in vacuo. The residue is purified by flash column chromatography, eluting with a gradient of 1-5% of methanol in dichloromethane to provide the desired compound as a pale-yellow solid (219 mg, 74% yield). ¹HNMR (300 MHz, DMSO-d₆) δ 11.57 (s, 1H), 8.75-9.02 (m, 1H), 7.53-7.80 (m, 3H), 7.35 (s, 1H), 6.83-7.20 (m, 2H), 6.38-6.59 (m, 1H), 5.39-5.66 (m, 1H), 4.14-4.64 (m, 1H), 3.87 (s, 3H), 2.91-3.26 (m, 2H), 1.96-2.37 (m, 3H), 1.65-1.99 (m, 3H), 1.13-1.66 (m, 8H). Anal. Calcd. For C₃₁H₃₃N₅O₅S•0.3 CH₂Cl₂: C, 61.31; H, 5.52; N, 11.42. Found: C, 61.18; H, 5.59; N, 11.29. LCMS ESI (M+H⁺): 588.20.

### Preparation of 3-cyclopentyl-N-[(4-methoxy-1H-indol-2-yl)carbonyl]-L-alanyl-N¹-methoxy-N¹-methyl-3-[(3S)-2-oxopyrrolidin-3-yl]-L-alaninamide

The material was prepared in a similar manner to N-{(1S)-1-cyclopentyl-2-[((1S)-2-[methoxy(methyl)amino]-2-oxo-1-{[(3S)-2-oxopyrrolidin-3-yl]methyl}ethyl)amino]-2-oxoethyl}-4-methoxy-1H-indole carboxamide (as described above) starting from tert-butyl ((1S)-2-[methoxy(methyl)amino]-2-oxo-1-{[(3S)-2-oxopyrrolidin-3-yl]methyl}ethyl)carbamate (268 mg, 0.85 mmol) except that Boc-p-cyclopentyl-L-alanine (200 mg, 0.777 mmol) was used in place of the N-Boc-L-cyclopentylglycine to give the desired compound as a white solid (287 mg, 70% yield). ¹H NMR (400 MHz, CDCl₃) δ 10.01 (bs, 1H), 8.05 (bs, 1H), 7.17 (t, J=8.1 Hz, 1H), 7.08 (d, J=1.5 Hz, 1H), 7.03 (t, J=8.3 Hz, 1H), 6.84 (d, J=8 Hz, 1H), 6.49 (d, J=7.6 Hz, 1H), 5.95 (bs, 1H), 5.03 (m, 1H), 4.93 (m, 1H), 3.95 (s, 3H), 3.82 (s, 3H), 3.26 (s, 3H), 3.22 (m, 2H) 2.43 (m, 2H), 2.17 (m, 1H), 1.95-1.70 (m, 8H), 1.60-1.40 (m, 3H), 1.14 (m, 2H). LCMS ESI (M+H⁺) 528.2, (M+Na⁺) 550.2.

### Preparation of N-{(1S)-2-[((1S)-2-(1,3-benzothiazol-2-yl)-2-oxo-1-{[(3S)-2-oxopyrrolidin-3-yl]methyl}ethyl)amino]-1-(cyclopentylmethyl)-2-oxoethyl}-4-methoxy-1H-indole-2-carboxamide

The title compound is prepared in a similar manner as described above for N-{(1S)-2-[((1S)-2-(1,3-benzothiazol-2-yl)-2-oxo-1-{[(3S)-2-oxopyrrolidin-3-yl]methyl}ethyl)amino]-1-cyclopentyl-2-oxoethyl}-4-methoxy-1H-indole-2-carboxamide, from benzothiazole (928 mg, 6.86 mmol), n-butyl lithium (2.5 M in hexanes, 2.74 mL, 6.86 mmol) and 3-cyclopentyl-N-[(4-methoxy-1H-indol-2-yl)carbonyl]-L-alanyl-N¹-methoxy-N¹-methyl-3-[(3S)-2-oxopyrrolidin-3-yl]-L-alaninamide (181 mg, 0.34 mmol) to give the desired title compound as an off white solid (105 mg, 51% yield). ¹H NMR (400 MHz, CDCl₃) δ 9.45 (s, 1H), 8.52 (d, J=7 Hz, 1H), 8.08 (m, 1H), 7.97 (m, 1H), 7.51 (m, 2H), 7.18 (m, 1H), 7.09 (d, J=1.5 Hz, 1H), 7.00 (d, J=8.4 Hz, 1H), 6.89 (d, J=8 Hz, 1H), 6.49 (d, J=7.8 Hz, 1H), 6.00 (s, 1H), 5.73 (m, 1H), 4.84 (m, 1H), 3.94 (s, 3H), 3.33 (m, 2H), 2.66 (m, 1H), 2.51 (m, 1H), 2.22 (m, 2H), 2.05-1.80 (m, 7H), 1.60-1.45 (m, 3H), 1.17 (m, 2H). LCMS ESI (M+H⁺) 602.1, (M+Na⁺) 624.1.

### Preparation of 3-cyclopropyl-N-(pyridine-2-ylcarbonyl)-L-alanyl-N¹-methoxy-N¹-methyl-3-[(3S)-2-oxopyrrolidin-3-yl]-L-alaninamide

tert-butyl ((1S)-2-[methoxy(methyl)amino]-2-oxo-1-{[(3S)-2-oxopyrrolidin-3-yl]methyl}ethyl)carbamate is deprotected using HCl in dioxane as described above and the resulting amine is coupled with Boc-p-cyclopropyl-L-alanine using hydroxybenzotriazole (˜1 equivalent) and 1-(3-dimethylaminopropyl)-3-ethylcarbodiimide hydrochloride (˜1.2 equivalents) and N-methylmorpholine in dichloromethane analogous to the methods described above to provide N-(tert-butoxycarbonyl)-3-cyclopropyl-L-alanyl-N¹-methoxy-N¹-methyl-3-[(3S)-2-oxopyrrolidin-3-yl]-L-alaninamide. The Boc group of N-(tert-butoxycarbonyl)-3-cyclopropyl-L-alanyl-N¹-methoxy-N¹-methyl-3-[(3S)-2-oxopyrrolidin-3-yl]-L-alaninamide (426.5 mg, 1.0 mmol) was removed (HCl in dioxane) and the resulting amine was coupled with pyridine-2-carboxylic acid (129 mg, 1.05 mmol) in the presence of 1-(3-dimethylaminopropyl)-3-ethylcarbodiimide hydrochloride (201 mg, 1.05 mmol), N,N-dimethylaminopyridine (12 mg, 0.1 mmol), N-methylmorpholine (0.55 mL, 5.0 mmol) in dichloromethane (5 mL) to give the desired compound as a white solid (272 mg, 63% yield). 1H NMR (400 MHz, CDCl₃) δ 8.65 (d, J=8 Hz, 1H), 8.58 (d, J=1.6 Hz, 1H), 8.17 (d, J=7.8 Hz, 1H), 7.84 (td, J=7.7, 1.7 Hz, 1H), 7.45-7.41 (m, 2H), 5.70 (s, 1H), 4.97 (m, 1H), 4.74 (m, 1H), 3.81 (s, 3H), 3.30 (m, 2H), 3.20 (s, 3H), 2.46 (m, 2H), 2.18 (m, 1H), 1.88-1.73 (m, 4H), 0.85 (m, 1H), 0.50 (m, 2H), 0.17 (m, 2H). LCMS ESI (M+H⁺) 432.1, (M+Na⁺) 454.1.

### Preparation of N-[(1S)-2-[((1S)-2-(1,3-benzothiazol-2-yl)-2-oxo-1-{[(3S)-2-oxopyrrolidin-3-yl]methyl}ethyl)amino]-1-(cyclopropylmethyl)-2-oxoethyl]pyridine-2-carboxamide

The title compound was prepared in a similar manner as N-{(1S)-2-[((1S)-2-(1,3-benzothiazol-2-yl)-2-oxo-1-{[(3S)-2-oxopyrrolidin-3-yl]methyl}ethyl)amino]-1-cyclopentyl-2-oxoethyl}-4-methoxy-1H-indole-2-carboxamide (described above) starting from benzothiazole (669 mg, 4.95 mmol), n-butyl lithium (2.5 M in hexanes, 1.98 mL, 4.95 mmol) and 3-cyclopropyl-N-(pyridine-2-ylcarbonyl)-L-alanyl-N¹-methoxy-N¹-methyl-3-[(3S)-2-oxopyrrolidin-3-yl]-L-alaninamide (178 mg, 0.41 mmol) to give the desired compound as a white solid (123 mg, 59% yield). 1H NMR (400 MHz, CDCl₃) δ 8.68 (d, J=8.3 Hz, 1H), 8.58 (d, J=4 Hz, 1H), 8.29 (d, J=6.8 Hz, 1H), 8.15 (d, J=7.8 Hz, 1H), 8.11-8.09 (m, 1H), 7.99-7.97 (m, 1H), 7.82 (td, J=7.7, 1.8 Hz, 1H), 7.54 (m, 2H), 7.42 (m, 1H), 5.77 (m, 1H), 5.71 (bs, 1H), 4.81 (m, 1H), 3.38 (m, 2H), 2.61 (m, 2H), 2.29-2.14 (m, 2H), 2.04 (m, 1H), 1.90-1.80 (m, 2H), 0.87 (m, 1H), 0.51 (m, 2H), 0.19 (m, 2H). LCMS ESI (M+H⁺) 506.1, (M+Na⁺) 528.1.

The synthesis of the compound filibuvir, (2R)-2-cyclopentyl-2-[2-(2,6-diethylpyridin-4-yl)ethyl]-5-[(5,7-dimethyl-[1,2,4]triazolo[1,5-a]pyrimidin-2-yl)methyl]-4-hydroxy-3H-pyran-6-one, an HCV polymerase inhibitor, is described in three back-to-back publications. For part I, see: R. A. Singer et al. Org. Process Res. Dev. 2014, 18, 26. For part II, see: Z. Peng, J. A. Ragan et al. Org. Process Res. Dev. 2014, 18, 36. For the discovery synthesis of filibuvir, see: H. Li et al. J. Med. Chem. 2009, 52, 1255.

Nelfinivir, (3S,4aS,8aS)—N-tert-butyl-2-[(2R,3R)-2-hydroxy-3-[(3-hydroxy-2-methylbenzoyl)amino]-4-phenylsulfanylbutyl]-3,4,4a,5,6,7,8,8a-octahydro-1H-isoquinoline-3-carboxamide, an antiretroviral protease inhibitor, can be prepared according to methods described in BA. Dressman et al., WO 9509843; and U.S. Pat. No. 5,484,926 (1995, 1996 both to Agouron).

Ruprintrivir, can be prepared as described in Example 17 of U.S. Pat. No. 6,995,142 and by Dragovich, P. S. et al. Structure-based design, synthesis, and biological evaluation of irreversible human rhinovirus 3C protease inhibitors. 3. Structure-activity studies of ketomethylene-containing peptidomimetics. J Med Chem, 1999, 42: 1203-1212; and in Lin, D. et al. Improved synthesis of rupintrivir; Science China Chemistry, June 2012 Vol. 55 No. 6: 1101-1107 doi: 10.1007/s11426-011-4478-5.

(R)-3-((2S,3S)-2-Hydroxy-3-{[1-(3-hydroxy-2-methyl-phenyl)-methanoyl]-amino}-4-phenyl-butanoyl)-5,5-dimethyl-thiazolidine-4-carboxylic acid allylamide; can be prepared according to the method described in U.S. Pat. Nos. 6,953,858 and 7,169,932 as well as WO 2005/054214 and WO 2005/054187.

### Docking Experiments

### Methods:

Homology Modeling. The sequence of 3C-like proteinase in SARS and COVID-19 can be found in references from the RCSB (e.g., 3IWM)¹ and the NCBI (e.g., Reference Sequence: YP_009725301.1NCBI)².

### SARS 3C Protease Sequence (PDB 3IWM):

SGFRKMAFPSGKVEGCMVQVTCGTTTLNGLWLDDTVYCPRHVICTAEDML
NPNYEDLLIRKSNHSFLVQAGNVQLRVIGHSMQNCLLRLKVDTSNPKTPK
YKFVRIQPGQTFSVLACYNGSPSGVYQCAMRPNHTIKGSFLNGSCGSVGF
NIDYDCVSFCYMHHMELPTGVHAGTDLEGKFYGPFVDRQTAQAAGTDTTI
TLNVLAWLYAAVINGDRWFLNRFTTTLNDFNLVAMKYNYEPLTQDHVDIL
GPLSAQTGIAVLDMCAALKELLQNGMNGRTILGSTILEDEFTPFDVVRQC
SGVTFQ

### New Wuhan Coronavirus SARS-CoV-2 Sequence (Same Section):

SGFRKMAFPSGKVEGCMVQVTCGTTTLNGLWLDDVVYCPRHVICTSEDML
NPNYEDLLIRKSNHNFLVQAGNVQLRVIGHSMQNCVLKLKVDTANPKTPK
YKFVRIQPGQTFSVLACYNGSPSGVYQCAMRPNFTIKGSFLNGSCGSVGF
NIDYDCVSFCYMHHMELPTGVHAGTDLEGNFYGPFVDRQTAQAAGTDTTI
TVNVLAWLYAAVINGDRWFLNRFTTTLNDFNLVAMKYNYEPLTQDHVDIL
GPLSAQTGIAVLDMCASLKELLQNGMNGRTILGSALLEDEFTPFDVVRQC
SGVTFQ

A homology model was built from a crystal structure of SARS 3C-like protease in Pfizer's database using Schrödinger's PRIME³. Minimization of the homology model in complex with ligands was used to remove clashes with ligands containing benzothiazole ketones or a benzyl side chains after examining the protein conformations of other SARS 3C-like crystal structures with these ligand moieties. Relaxation of residues in the 185-190 loop, His41 and Met49 to led to three differently minimized versions of the homology model. The catalytic Cys was mutated to Gly (C145G) to facilitate AGDOCK⁵ core docking and subsequent scoring without a clash with the catalytic Cys.

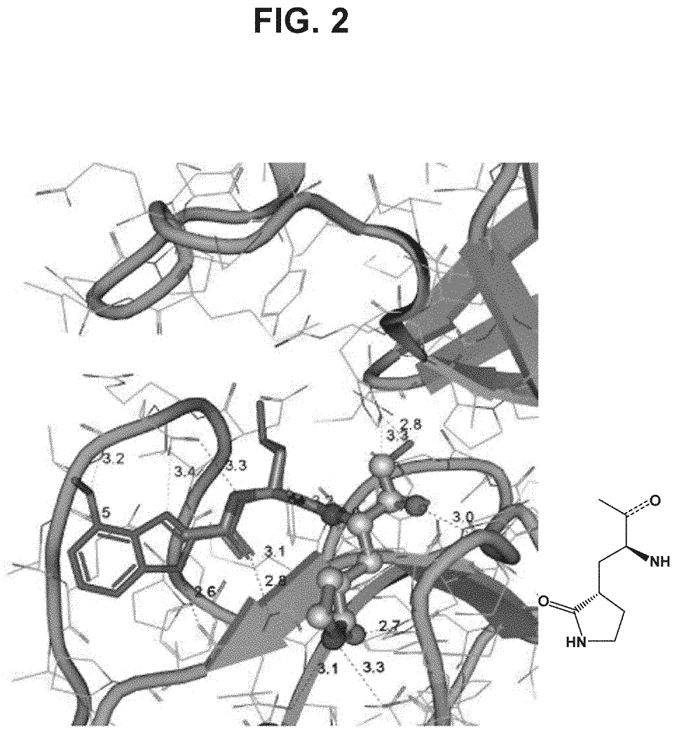

Docking: Nine (9) compounds were docked into the homology models using core docking⁴ with AGDOCK⁵. The docking was performed without forming the protein-ligand covalent bond. Instead, a common core that included the lactam side chain and reactive ketone was identified in the ligands and held fixed in the crystal structure orientation as a mimic of covalent docking (See FIG. 2). The affinity measure for AGDOCK core docking was HT Score⁶.

### METHOD REFERENCES

1. http://www.rcsb.org/structure/3IWM
2. https://www.ncbi.nilm.nih.gov/protein/YP_009725301.1
3. Schródinger Release 2019-1: Prime, Schrödinger, LLC, New York, N.Y., 2019.
4. Daniel K. Gehlhaar, Gennady M. Verkhivker, Paul A. Rejto, Christopher J. Sherman, David R. Fogel, Lawrence J. Fogel, Stephan T. Freer, Molecular recognition of the inhibitor AG-1343 by HIV-1 protease: conformationally flexible docking by evolutionary programming, Chemistry & Biology, Volume 2, Issue 5, 1995, Pages 317-324.
5. Daniel K. Gehlhaar, Djamal Bouzida, and Paul A. Rejto, Reduced Dimensionality in Ligand—Protein Structure Prediction: Covalent Inhibitors of Serine Proteases and Design of Site-Directed Combinatorial Libraries Rational Drug Design. Jul. 7, 1999, 292-311.
6. Tami J. Marrone, Brock A. Luty, Peter W. Rose, Discovering high-affinity ligands from the computationally predicted structures and affinities of small molecules bound to a target: A virtual screening approach. Perspectives in Drug Discovery and Design 20, 209-230 (2000).

### Results:

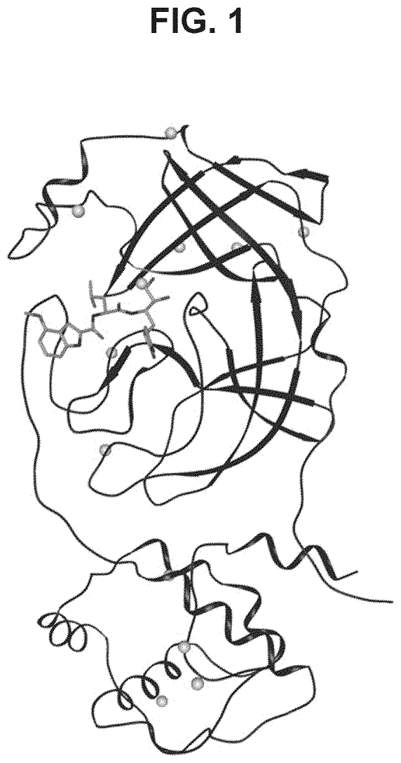

Homology model: The sequence homology between SARS-CoV and SARS-CoV-2 is 96.1%. There are 12 of 306 residues that are different (T35V, A46S, S65N, L86V, R88K, S94A, H134F, K180N, L202V, A267S, T285A & I286L highlighted in FIG. 1) which translates to 96.1% identity.

The ligand associated with the crystal structure used to build the homology model is Compound B, N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide.

The amino acid residue nearest to Compound B, N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide, that differed between SARS 3C-like protease and SARS-CoV-2 3C-like protease model is A46S, and the minimum distance from Cₐₗₚₕₐ to ligand is 8.3 Å. Other residues are between 11 Å and 38 Å from the nearest atom in Compound B.

TABLE 1
Approximate distances from Cₐₗₚₕₐ atoms in SARS-CoV-2
to Compound B, N-((1S)-1-{[((1S)-3-hydroxy-2-oxo-1-
{[(3S)-2-oxopyrrolidin-3-yl]methyl}propyl)amino]
carbonyl}-3-methylbutyl)-4-methoxy-1H-indole-2-carboxamide

| | Distance to Nearest Atom |
|---|---|
| SARS-CoV to SARS-CoV-2 Amino Acid | in Compound B |
| Residue Changes | (Angstroms) |
| T35V | ~19 |
| A46S | ~8 |
| S65N | ~16 |
| L86V | ~11 |
| R88K | ~15 |
| S94A | ~24 |
| H134F | ~14 |
| K180N | ~13 |
| L202V | ~27 |
| A267S | ~38 |
| T285A | ~34 |
| I286L | ~31 |

FIG. 1. FIG. 1 depicts the residue differences between SARS-CoV and SARS-CoV-2. The location of the residue changes are indicated with grey spheres in this ribbon depiction of SARS-CoV-2 homology model. The Compound B, N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide, location (upper left side) is shown in stick format. The approximate distance between the C-alpha of a SARS-CoV-2 amino acid residue and the closest atom in the Compound B, N-((1S)-1-{[((1S)-3-hydroxy-2-oxo-1-{[(3S)-2-oxopyrrolidin-3-yl]methyl}propyl)amino] carbonyl}-3-methylbutyl)-4-methoxy-1H-indole-2-carboxamide, is shown in Table 1, above.

### Docking Results:

The approximately 96% homology of SARS-CoV-2 3CL to SARS-CoV 3CL and the similarity between ligands allows a comparison of the RMSD between the peptide backbone of xtal ligand in SARS-CoV (see FIG. 2) and the docked ligand in the SARS-CoV-2 3CL model. The core-docked ligand RMSD to the peptide backbone did not differ by more than 0.32 Å (average 0.28 Å). See FIG. 2 for an example. In the case of Compound B, N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide; the RMSD for the whole molecule was 0.37 Å.

FIG. 2. Binding site of homology model of SARS-CoV-2 3CL with a core-docked ligand (Compound B, N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide) present. Part of the crystal structure of Compound B, N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide; (peptide backbone, lactam side chain and attacked ketone) was used to measure the RMSD of the different ligands to that backbone (grey carbons, thick stick). The core used for core docking is shown as 11 heavy atoms in ball representation (light carbons) and in the inset chemical structure. Distances shown in Angstroms.

The docking results in Table 2 below indicate that several of the compounds have predicted affinities (ΔG_{bind}, kcal/mol) that are generally commensurate with target recognition and binding. The method used to estimate ΔG was the HTS scoring function.⁶ The effective potency can differ from the ΔG binding terms depending on several factors such as cell uptake, efflux, cofactor competition or substrate competition.

TABLE 2

| | Predicted | |
|---|---|---|
| | ΔG_{bind} | |
| Compound | (kcal/mol) | Chemical Name of Docked Compounds |
| A | −8.9 | (3S)-3-({4-methyl-N-[(2R)-tetrahydrofuran-2-ylcarbonyl]-L- |
| | | leucyl}amino)-2-oxo-4-[(3S)-2-oxopyrrolidin-3-yl]butyl 2,6- |
| | | dichlorobenzoate |
| B | −9.5 | N-((1S)-1-{[((1S)-3-hydroxy-2-oxo-1-{[(3S)-2-oxopyrrolidin-3- |
| | | yl]methyl}propyl)amino] carbonyl}-3-methylbutyl)-4-methoxy- |
| | | 1H-indole-2-carboxamide |
| C | −10 | (3S)-3-({N-[(4-methoxy-1H-indol-2-yl)carbonyl]-L- |
| | | leucyl}amino)-2-oxo-4-[(3S)-2-oxopyrrolidin-3-yl]butyl |
| | | cyclopropanecarboxylate |
| D | −9.4 | N-((1S)-1-{[((1S)-3-hydroxy-2-oxo-1-{[(3S)-2-oxopyrrolidin-3- |
| | | yl]methyl}propyl)amino]carbonyl}pentyl)-4-methoxy-1H- |
| | | indole-2-carboxamide |
| E | −10 | N-((1S)-1-{[((1S)-3-hydroxy-2-oxo-1-{[(3S)-2-oxopyrrolidin-3- |
| | | yl]methyl}propyl)amino] carbonyl}-3,3-dimethylbutyl)-1H- |
| | | indole-2-carboxamide |
| F | −10.1 | N-((S)-2-(((S)-1-(benzo[d]thiazol-2-yl)-1-oxo-3-((S)-2- |
| | | oxopyrrolidin-3-yl)propan-2-yl)amino)-1-cyclopentyl-2- |
| | | oxoethyl)-4-methoxy-1H-indole-2-carboxamide |
| G | −10.4 | N-((S)-1-(((S)-1-(benzo[d]thiazol-2-yl)-1-oxo-3-((S)-2- |
| | | oxopyrrolidin-3-yl)propan-2-yl)amino)-3-cyclopentyl-1- |
| | | oxopropan-2-yl)-4-methoxy-1H-indole-2-carboxamide |
| H | −7.8 | N-((S)-1-(((S)-1-(benzo[d]thiazol-2-yl)-1-oxo-3-((S)-2- |
| | | oxopyrrolidin-3-yl)propan-2-yl)amino)-3-cyclopropyl-1- |
| | | oxopropan-2-yl)picolinamide |
| I | −9.8 | N-((S)-1-(((S)-4-hydroxy-3-oxo-1-((S)-2-oxopyrrolidin-3- |
| | | yl)butan-2-yl)amino)-1-oxo-3-phenylpropan-2-yl)-4-methoxy- |
| | | 1H-indole-2-carboxamide |

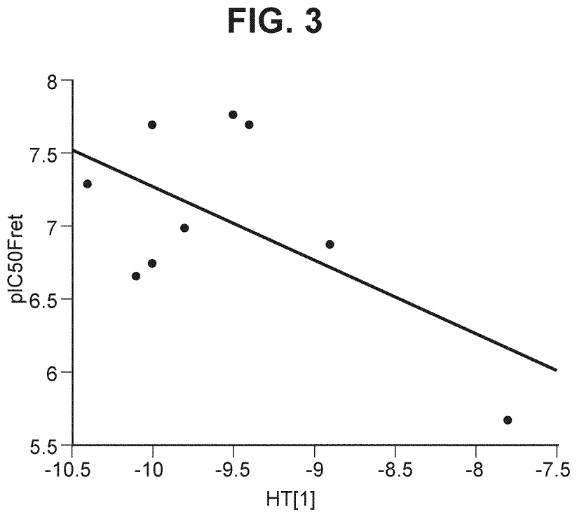

FIG. 3. Fit between predicted ΔG COVID-19 (HT) compared to FRET-based 1050 values against SARS. There is an R-sq of 0.35 and the fit is significant at the 90% confidence level (insignificant at 95% confidence level) for 9 compounds. The compounds described above are analyzed by a FRET biochemical assay and by in vitro virological assays using cell culture techniques.

Protection from SARS Infection: Neutral Red Endpoint

The ability of compounds to protect cells against infection by the SARS coronavirus is measured by a cell viability assay similar to that described in Borenfreund, E., and Puerner, J. 1985. Toxicity determined in vitro by morphological alterations and neutral red absorption Toxicology Letters. 24:119-124, utilizing neutral red staining as an endpoint. Briefly, medium containing appropriate concentrations of compound or medium only is added to Vero cells. Cells are infected with SARS-associated virus or mock-infected with medium only. One to seven days later, the medium is removed and medium containing neutral red is added to the test plates. Following incubation at 37° C. for two hours, cells are washed twice with PBS and a 50% EtOH, 1% acetic acid solution is added. The cells are shaken for 1 to 2 minutes and incubated at 37° C. for 5 to 10 minutes. The amount of neutral red is quantified spectrophotometrically at 540 nm. Data is expressed as the percent of neutral red in wells of compound-treated cells compared to neutral red in wells of uninfected, compound-free cells. The fifty percent effective concentration (EC50) is calculated as the concentration of compound that increases the percent of neutral red production in infected, compound-treated cells to 50% of that produced by uninfected, compound-free cells. The 50% cytotoxicity concentration (CC50) is calculated as the concentration of compound that decreases the percentage of neutral red produced in uninfected, compound-treated cells to 50% of that produced in uninfected, compound-free cells. The therapeutic index is calculated by dividing the cytotoxicity (CC50) by the antiviral activity (EC50).

Protection from SARS-CoV-2 Infection: Glo Endpoint

The ability of compounds to protect cells against infection by the SARS-CoV-2 coronavirus can also be measured by a cell viability assay utilizing luciferase to measure intracellular ATP as an endpoint. Briefly, medium containing appropriate concentrations of compound or medium only is added to Vero cells. Cells are infected with SARS-CoV-2 virus or mock-infected with medium only. One to seven days later, the medium is removed and the amount of intracellular ATP is measured as per Promega Technical Bulletin No. 288: CellTiter-Glo® Luminescent Cell Viability Assay (Promega, Madison, Wis.). The CellTiter-Glo® reagent is added to the test plates and following incubation at 37° C. for 1.25 hours, the amount of signal is quantified using a luminometer at 490 nm. Data is expressed as the percent of luminescent signal from wells of compound-treated cells compared to the luminescent signal from wells of uninfected, compound-free cells. The fifty percent effective concentration (EC50) is calculated as the concentration of compound that increases the percent of the luminescent signal from infected, compound-treated cells to 50% of the luminescent signal from uninfected, compound-free cells. The 50% cytotoxicity concentration (CC50) is calculated as the concentration of compound that decreases the percentage of the luminescent signal from uninfected, compound-treated cells to 50% of the luminescent signal from uninfected, compound-free cells. The therapeutic index is calculated by dividing the cytotoxicity (CC50) by the antiviral activity (EC50).

### Cytotoxicity

The ability of compounds to cause cytotoxicity in cells is measured by a cell viability assay similar to that described in Weislow, O. S., Kiser, R., Fine, D. L., Bader, J., Shoemaker, R. H., and Boyd, M. R. 1989. New Soluble-Formazan Assay for HIV-1 Cytopathic Effects: Application to High-Flux Screening of Synthetic and Natural Products for AIDS-Antiviral Activity. Journal of the National Cancer Institute 81(08): 577-586, utilizing formazan as an endpoint. Briefly, Vero cells are resuspended in medium containing appropriate concentrations of compound or medium only. One to seven days later, XTT and PMS are added to the test plates and following incubation at 37° C. for two hours the amount of formazan produced is quantified spectrophotometrically at 540 nm. Data is expressed as the percent of formazan produced in compound-treated cells compared to formazan produced in wells of compound-free cells. The 50% cytotoxicity concentration (CC50) is calculated as the concentration of compound that decreases the percentage of formazan produced in uninfected, compound-treated cells to 50% of that produced in uninfected, compound-free cells.

Protection from SARS-CoV-2 Coronavirus Infection

The ability of compounds to protect cells against infection by SARS-CoV-2 is measured by a cell viability assay similar to that described in Weislow, O. S., Kiser, R., Fine, D. L., Bader, J., Shoemaker, R. H., and Boyd, M. R. 1989. New Soluble-Formazan Assay for HIV-1 Cytopathic Effects: Application to High-Flux Screening of Synthetic and Natural Products for AIDS-Antiviral Activity. Journal of the National Cancer Institute 81(08): 577-586, utilizing formazan as an endpoint. Briefly, medium containing appropriate concentrations of compound or medium only is added to MRC-5 cells. Cells are infected with human coronavirus SARS-CoV-2 or mock-infected with medium only. One to seven days later, XTI and PMS are added to the test plates and following incubation at 37° C. for two hours the amount of formazan produced is quantified spectrophotometrically at 540 nm. Data is expressed as the percent of formazan in wells of compound-treated cells compared to formazan in wells of uninfected, compound-free cells. The fifty percent effective concentration (EC50) is calculated as the concentration of compound that increases the percent of formazan production in infected, compound-treated cells to 50% of that produced by uninfected, compound-free cells. The 50% cytotoxicity concentration (CC50) is calculated as the concentration of compound that decreases the percentage of formazan produced in uninfected, compound-treated cells to 50% of that produced in uninfected, compound-free cells. The therapeutic index is calculated by dividing the cytotoxicity (CC50) by the antiviral activity (EC50).

### SARS-CoV-2 Coronavirus 3C Protease FRET Assay and Analysis

Proteolytic activity of SARS-CoV-2 Coronavirus 3CL protease is measured using a continuous fluorescence resonance energy transfer assay. The SARS-CoV-2 3CLpro FRET assay measures the protease catalyzed cleavage of TAMRA-SITSAVLQSGFRKMK-(DABCYL)-OH to TAMRA-SITSAVLQ and SGFRKMK(DABCYL)-OH. The fluorescence of the cleaved TAMRA (ex. 558 nm/em. 581 nm) peptide was measured using a TECAN SAFIRE fluorescence plate reader over the course of 10 min. Typical reaction solutions contained 20 mM HEPES (pH 7.0), 1 mM EDTA, 4.0 uM FRET substrate, 4% DMSO and 0.005% Tween-20. Assays were initiated with the addition of 25 nM SARS 3CL^{pro} (nucleotide sequence 9985-10902 of the Urbani strain of SARS coronavirus complete genome sequence (NCBI accession number AY278741)). Percent inhibition was determined in duplicate at 0.001 mM level of inhibitor. Data was analyzed with the non-linear regression analysis program Kalidagraph using the equation:

FU=offset+(limit)(1−e^{-(kobs)}t)

where offset equals the fluorescence signal of the uncleaved peptide substrate, and limit equals the fluorescence of fully cleaved peptide substrate. The kobs is the first order rate constant for this reaction, and in the absence of any inhibitor represents the utilization of substrate. In an enzyme start reaction which contains an irreversible inhibitors, and where the calculated limit is less than 20% of the theoretical maximum limit, the calculated kobs represents the rate of inactivation of coronavirus 3C protease. The slope (kobs/l) of a plot of kobs vs. [l] is a measure of the avidity of the inhibitor for an enzyme. For very fast irreversible inhibitors, kobs/l is calculated from observations at only one or two [l] rather than as a slope.

Alternatively, the compounds may be assessed using the SARS CoV-2 FRET Assay below.

### SARS CoV-2 Protease FRET Assay and Analysis

The proteolytic activity of the main protease, 3CLpro, of SARS-CoV-2 was monitored using a continuous fluorescence resonance energy transfer (FRET) assay. The SARS-CoV-2 3CLpro assay measures the activity of full length SARS-CoV-2 3CL protease to cleave a synthetic fluorogenic substrate peptide with the following sequence Dabcyl-KTSAVLQ-SGFRKME-Edans modelled on a consensus peptide. The fluorescence of the cleaved Edans peptide (excitation 340 nm/emission 490 nm) is measured using a fluorescence intensity protocol on a Flexstation reader (Molecular Devices). The fluorescent signal is reduced in the presence of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide, a potent inhibitor of SARS-CoV-2 3CL pro. The assay reaction buffer contained 20 mM Tris-HCl (pH 7.3), 100 nM NaCl, 1 mM EDTA, 5 mM TCEP and M peptide substrate. Enzyme reactions were initiated with the addition of 15 nM SARS-CoV-2 3CL protease and allowed to proceed for 60 min at 23° C. Percent inhibition or activity was calculated based on control wells containing no compound (0% inhibition/100% activity) and a control compound (100% inhibition/0% activity). IC₅₀ values were generated using a four-parameter fit model using ABASE software (IDBS). Kᵢ values were fit to the Morrison equation with the enzyme concentration parameter fixed to 15 nM, the Kₘ parameter fixed to 14 μM and the substrate concentration parameter fixed to 25 uM using Activity Base software (IDBS).

The compound (3S)-3-({4-methyl-N-[(2R)-tetrahydrofuran-2-ylcarbonyl]-L-leucyl}amino)-2-oxo-4-[(3S)-2-oxopyrrolidin-3-yl]butyl 2,6-dichlorobenzoate when evaluated in the above assay had an IC₅₀ of 17 nM (95% confidence interval of 16 nM to 18 nM with n=32) and a Ki of 2.71 nM (95% confidence interval of 2.29 nM to 3.13 nM with n=32). The compound N-((1S)-3-hydroxy-2-oxo-1-{[(3S)-2-oxopyrrolidin-3-yl]methyl}propyl)-N-[(4-methoxy-1H-indol-2-yl)carbonyl]-L-phenylalaninamide when evaluated in the above assay had an IC₅₀ of 375 nM (95% confidence interval of 260 nM to 489 nM with n=4) and a Ki of 139 nM (95% confidence interval of 65.8 nM to 212 nM with n=2). The compound (3S)-3-({N-[(4-methoxy-1H-indol-2-yl)carbonyl]-L-leucyl}amino)-2-oxo-4-[(3S)-2-oxopyrrolidin-3-yl]butyl cyclopropanecarboxylate when evaluated in the above assay had an IC₅₀ of 128 nM (95% confidence interval of 107 nM to 149 nM with n=5) and a Ki of 42.7 nM (95% confidence interval of 37.9 nM to 47.5 nM with n=2). The compound N-((1S)-1-{[((1S)-3-hydroxy-2-oxo-1-{[(3S)-2-oxopyrrolidin-3-yl]methyl}propyl)amino]carbonyl}-3,3-dimethylbutyl)-1H-indole-2-carboxamide when evaluated in the above assay had an IC₅₀ of 24 nM (95% confidence interval of 16 nM to 32 nM with n=6) and a Ki of 2.81 nM (95% confidence interval of 1.39 nM to 4.22 nM with n=4). The compound N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide when evaluated in the above assay had an IC₅₀ of 7 nM (95% confidence interval of 6 nM to 8 nM with n=7) and a Ki of 0.27 nM (95% confidence interval of 0.17 nM to 0.37 nM with n=6). The compound N—((S)-1-(((S)-1-(benzo[d]thiazol-2-yl)-1-oxo-3-((S)-2-oxopyrrolidin-3-yl)propan-2-yl)amino)-3-cyclopropyl-1-oxopropan-2-yl)picolinamide when evaluated in the above assay had an IC₅₀ of 8402 nM (95% confidence interval of 4396 nM to 12407 nM with n=3) and a Ki of 3048 nM (n=1). The compounds AG-0011859, filibuvir, nelfinavir and ruprintrivir all had IC50s of >30 M when assessed in the assay above.

N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide was evaluated against 3CLpro from a variety of other coronaviruses representing alpha, beta and gamma groups of coronaviridae, using biochemical Fluorescence Resonance Energy Transfer (FRET) protease activity assays. The assays are analogous to the FRET assay above and can employ the full-length protease sequences from the indicated viruses. N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide demonstrated potent inhibitory activity against all tested coronavirus 3CLpro including members of alpha-coronaviruses (NL63-CoV, PEDV-CoV-2, FIPV-CoV-2), beta-coronaviruses (HKU4-CoV, HKU5-CoV, HKU9-CoV, MHV-CoV, OC43-CoV, HKU1-CoV), and gamma-coronavirus (IBV-CoV-2), with Ki values and tested enzyme concentrations included in Table 3. This inhibitory activity is restricted to coronavirus 3CL proteases as N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide was inactive against a panel of human proteases and HIV protease. N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide showed detectable activity against human cathepsin B but with a 1000-fold margin compared to 3CLpro (Table 4). These data collectively support N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide as a pan coronavirus 3 CL protease inhibitor.

TABLE 3
Activity of N-((1S)-1-{[((1S)-3-hydroxy-2-oxo-1-
{[(3S)-2-oxopyrrolidin-3-yl]methyl} propyl)amino]
carbonyl}-3-methylbutyl)-4-methoxy-1H-indole-2-carboxamide
against 3CLpro of coronaviruses

| | | |
|---|---|---|
| Virus | Kᵢ (nM) | [E]_{T} (nM) |

Alpha-CoV

| | | |
|---|---|---|
| NL63-CoV | 0.8 ± 0.5 | 170 ± 4 |
| 229E-CoV-2 | 1.5 ± 0.8 | 118 ± 3 |
| PEDV-CoV-2 | 0.3 ± 0.1 | 40 ± 1 |
| FIPV-CoV-2 | 0.1 ± 0.1 | 37 ± 1 |

Beta-CoV

| | | |
|---|---|---|
| HKU1-CoV | 0.9 ± 0.2 | 57 ± 1 |
| HKU4-CoV | 0.03 ± 0.08 | 60 ± 1 |
| HKU5-CoV | 0.03 ± 0.1 | 75 ± 1 |
| HKU9-CoV | 0.8 ± 0.6 | 264 ± 5 |
| MHV-CoV | 1.2 ± 0.9 | 75 ± 4 |
| OC43-CoV | 0.5 ± 0.1 | 52 ± 1 |

Gamma-CoV

| | | |
|---|---|---|
| IBV-CoV-2 | 4.0 ± 0.4 | 30 ± 1 |

TABLE 4
Activity of N-((1S)-1-{[((1S)-3-hydroxy-2-oxo-
1-{[(3S)-2-oxopyrrolidin-3-yl]methyl}propyl)
amino]carbonyl}-3-methylbutyl)-4-methoxy-1H-
indole-2-carboxamide against human proteases and HIV protease

| Protease | IC₅₀ μM |
|---|---|
| SAR-Cov2 3CLpro | 0.00692 |
| Human Cathepsin B | 6.12 |
| Human Elastase | >33.3 |
| Human Chymotrypsin | >100 |
| Human Thrombin | >100 |
| Human Caspase 2 | >33.3 |
| Human Cathepsin D | >11.1 |
| HIV-1 protease | >11.1 |

Thermal Shift Binding Data of N-((1S)-1-{[((1S)-3-hydroxy-2-oxo-1-{[(3S)-2-oxopyrrolidin-3-yl]methyl} propyl)amino] carbonyl}-3-methylbutyl)-4-methoxy-1H-indole-2-Carboxamide with SARS-CoV-2 3CLpro Indicates Tight and Specific Binding to SARS-CoV-2 3CL in vitro

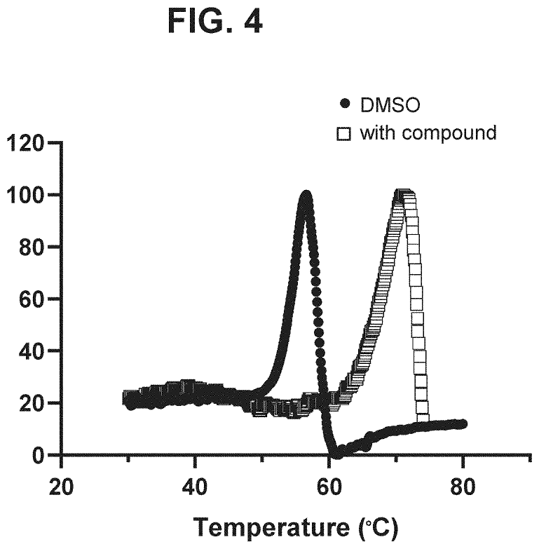

In view of the ability of N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide to potently inhibit SARS-CoV-2 3CLpro with a Ki value of 0.27 nM further studies were undertaken. Studies of the X-ray co-crystal structure of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide and SARS-CoV-2 3CLpro is consistent with the compound binding to the 3CL enzyme with a covalent and reversible interaction at catalytic cysteine residue of the active site, thus inhibiting the activity of the 3CLpro. A thermal-shift assay was also used to evaluate the direct binding between N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide and its target protein, SARS-CoV-2 3CLpro. The melting temperature of SARS-CoV-2 3CLpro was shifted by 14.6° C. upon binding of N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide, from 55.9+/−0.11° C. (n=16) to 70.5+/−0.12° C. (n=8). The melting temperature (Tm) was calculated as the mid-log of the transition phase from the native to the denatured protein using a Boltzmann model in Protein Thermal Shift Software v1.3. These data support tight and specific binding of N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide to SARS-CoV-2 3CLpro (see FIG. 4) and, thereby, provide further evidence for the molecular mechanism of this compound as an inhibitor of SARS-CoV-2 3CLpro.

SARS-CoV-2 cellular antiviral activity is inhibited by N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide in vitro.

The antiviral activity of N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide against SARS-CoV-2 in cell culture was evaluated with a cytopathic effect (CPE) assay using either VeroE6 cells enriched for ACE2 (VeroE6-enACE2) receptor or VeroE6 cells constitutively expressing EGFP (VeroE6-EGFP). These cell lines were infected with the SARS-CoV-2 Washington strain 1 or the BetaCov GHB-03021/2020 strain, respectively, which have identical 3CLpro amino acid sequences. N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide protected the cells from the viral CPE at 39.7 μM and 88.9 μM, respectively (EC₅₀, Table 5). However, Vero cells express high levels of the efflux transporter P-gp (also known as MDR1 orABCB1), of which N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide is a known substrate. Therefore, the assays were repeated in the presence of a P-gp efflux inhibitor, CP-100356, 4-(3,4-Dihydro-6,7-dimethoxy-2(1H)-isoquinolinyl)-N-2[2-(3,4-dimethoxyphenyl)ethyl]-6,7-dimethoxy-2-quinazolinamine. N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide exhibited a 117 to 173-fold increase in activity in the presence of 2 μM P-gp inhibitor, with EC₅₀ values of 0.23 μM in VeroE6-enACE2 cells and 0.76 μM in the VeroE6-EGFP cells (Table 5). The P-gp inhibitor alone had no antiviral or cytotoxic activity at these concentrations and did not cause cytotoxicity in the presence the protease inhibitor. There was a steep response to increasing doses of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide, with a ˜2-3 fold difference between EC₅₀ and EC₉₀ (i.e. between the 50% and 90% effective concentrations) in both cell types (EC₅₀=0.48 uM in VeroE6-enACE2 cells and EC₉₀=1.6 uM in VeroE6-EGFP cells in the presence of the P-gp inhibitor). When lung cell lines were tested for antiviral potency in the presence and absence of P-gp inhibitor (A549-ACE2 and MRC5) no significant difference in antiviral potency was observed (Table 5). Additionally, the EC₅₀ and EC₉₀ values in both veroE6 cell lines with 2 uM P-gp are similar to those obtained using different assay methods with different cell types, including by detecting viral protein in A549-ACE2 cells as well as using plaque assays in polarized human airway epithelial cells, where Pg-p expression is lower.

TABLE 5
In vitro antiviral activity, cytotoxicity and therapeutic index
(TI) of N-((1S)-1-{[((1S)-3-hydroxy-2-oxo-1-{[(3S)-
2-oxopyrrolidin-3-yl]methyl} propyl)amino]carbonyl}-3-
methylbutyl)-4-methoxy-1H-indole-2-carboxamide

| | | | | EC₅₀ μM | CC₅₀ μM | |
|---|---|---|---|---|---|---|
| | | Efflux | | GeoMean | GeoMean | TI |
| Cells | Virus | Inhibitor | | (95% Cl) | (95% Cl) | CC₅₀/EC₅₀ |
| Vero E6- | SARS2 | 0 | | 38.7 | >100 | >2.5 |
| enACE2 | Washington1 | | | (29.8, 52.9) | (ND) | |
| | | | | n = 12 | n = 9 | |
| | | 0.5 | μM | 3.0 | >100 | >42 |
| | | | | (1.13, 7.67) | (ND) | |
| | | | | n = 7 | n = 9 | |
| | | 2 | μM | 0.23 | >100 | >436 |
| | | | | (0.13, 0.41) | (ND) | |
| | | | | n = 6 | n = 6 | |
| Vero E6-EGFP | SARS2 | 0 | | 88.9 | >100 | >2.6 |
| | BetaCov | | | (76.8, 103) | (ND) | |
| | GHB- | | | n = 10 | n = 8 | |
| | 03021/2020 | 0.5 | μM | 10.0 | >100 | >20.6 |
| | | | | (3.93, 25.7) | (ND) | |
| | | | | n = 10 | n = 1 | |
| | | 2 | μM | 0.76 | >50 | >69 |
| | | | | (0.45, 1.14) | (ND) | |
| | | | | n = 4 | n = 4 | |

| | | | | | |
|---|---|---|---|---|---|
| MRC-5 | HCOV-229E | 0 | 0.069 | >100 | >510 |
| | | | (0.056, 0.085) | (ND) | |
| | | | n = 7 | n = 5 | |

| | | | | |
|---|---|---|---|---|
| 0.5 | μM | 0.080 | >100 | >770 |
| | | (0.017, 0.37) | (ND) | |
| | | n = 3 | n = 3 | |

The potency of N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide in combination with either azithromycin or remdesivir for antiviral activity against SARS-CoV-2 in VeroE6 cells. In brief, VeroE6 cells that are enriched for hACE2 expression were batched innoculated with SARS-CoV-2 (USA_WA1/2020) at a multiplicity of infection of 0.002 in a BSL-3 lab. Virus innoculated cells are then added to assay ready compound plates at a density of 4,000 cells/well. Following a 3-day long incubation, a time at which virus-induced cytopathic effect is 95% in the untreated, infected control conditions, cell viability was evaluated using Cell Titer-Glo (Promega), according to the manufacturer's protocol, which quantitates ATP levels. Cytotoxicity of the compounds was assessed in parallel non-infected cells.

To examine whether combinatory treatments have synergistic or additive effects, each compound is tested at concentrations in a dose matrix. Chalice Analyzer was used to calculate the Loewe additivity and excess models. The Loewe excess is commonly used to indicate the excess percent inhibition; the excess percent inhibition is calculated by deducting the expected percent inhibition values of various combinations, assuming nonsynergy pairing in various models, from the experimental percent inhibition values. These data allowed calculation of the isobologram, synergy score, and best combination index (CI) for each pair. In general, synergy scores of >1 and CI of <1 indicate that a combination treatment has a synergistic effect; a synergy score of 1 and a CI of 1 indicate that a combination treatment has only an additive effect. Antimicrob Agents Chemother. 2015 April; 59(4): 2086-2093. doi: 10.1128/AAC.04779-14

To assess whether synergy could be achieved at high inhibition levels, the isobologram level was set at 0.9 to capture meaningful synergy with a 90% viral reduction (equivalent to a 1-log₁₀ reduction).

The combination of N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide plus azithromycin generated synergy, with a synergy score of 3.76 and a CI of 0.4. The observed synergy was not due to cytotoxicity, as there was no significant cytotoxicity for all the combinations tested. The combination of N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide and remdesivir demonstrated additivity, with a synergy score of 5.1 and a CI of 0.21. The observed synergy may potentially be used to reduce the doses and therefore to increase the safety margins of inhibitors to achieve a therapeutic window in vivo. Additionally, combination therapy could be utilized to minimize drug resistance.

Additional studies were carried out to further assess the potential for antiviral combination benefit of N-((1S)-1-{[((1S)-3-hydroxy-2-oxo-1-{[(3S)-2-oxopyrrolidin-3-yl]methyl} propyl)amino] carbonyl}-3-methylbutyl)-4-methoxy-1H-indole-2-carboxamide in combination with remdesivir.

Combinations of antiviral agents, especially those targeting different steps in the virus replication cycle, are a frequently employed therapeutic strategy in treating viral diseases. As N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide and remdesivir, a nucleoside RNA-dependent RNA polymerase inhibitor, target different steps in the viral replication cycle, the antiviral activity of the two compounds was evaluated alone and in combination using HeLa-ACE2 cells. Viral proteins were detected in this assay using convalescent human polyclonal sera from two different COVID-19 patients. N-((1S)-1-{[((1S)-3-hydroxy-2-oxo-1-{[(3S)-2-oxopyrrolidin-3-yl]methyl} propyl)amino] carbonyl}-3-methylbutyl)-4-methoxy-1H-indole-2-carboxamide (designated compound 1 in table 6) alone inhibited SARS-CoV-2 replication with an average EC₅₀ of 0.14 μM and EC₉₀ of 0.40 μM; whereas remdesivir had an average EC₅₀ of 0.074 μM and EC₉₀ of 0.17 μM (Table 6).

TABLE 6
In vitro activity of N-((1S)-1-{[((1S)-3-hydroxy-
2-oxo-1-{[(3S)-2-oxopyrrolidin-3-yl]methyl}propyl)amino]carbonyl}-3-
methylbutyl)-4-methoxy-1H-indole-2-carboxamide (compound
1) and remdesivir in HeLa-ACE2 cells

| Compound | EC₅₀ (μM) | EC₉₀ (μM) | n |
|---|---|---|---|
| compound 1 | 0.144 | 0.398 | 3 |
| | (0.0738-0.280) | (0.143-1.11) | |
| Remdesivir | 0.0739 | 0.168 | 4 |
| | (0.629-0.0867) | (0.110-0.256) | |

Combination studies were performed using a drug testing matrix and the data for the drug combination were analyzed using reference models (Loewe, Bliss, HSA) to classify the effects of the drug combination as either additive, synergistic or antagonistic (isobologram, synergy scores, and combination indices). In general, a synergy score of >1 and a combination index of <1 indicate that the combination treatment has a synergistic effect (Yeo et al, 2015). To assess whether synergy could be achieved at high inhibition levels, the isobologram level was set at 0.9 to capture meaningful synergy with a 90% viral reduction (equivalent to a 1 log₁₀ reduction).

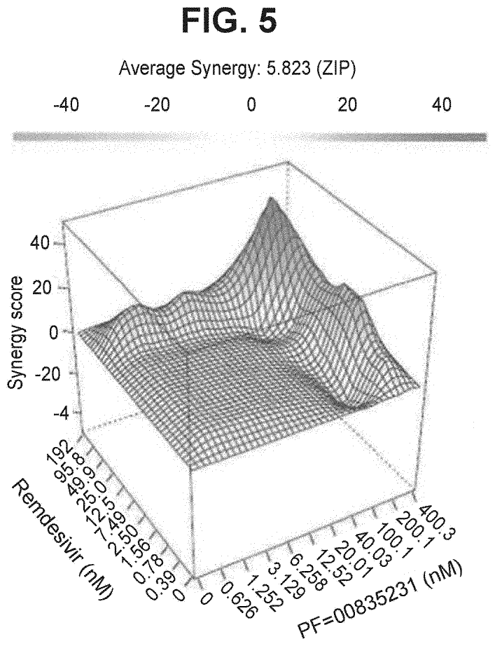

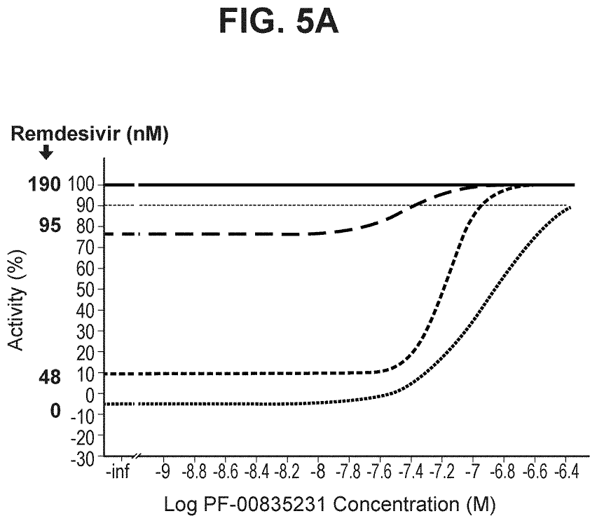

As summarized in Table 7, the combination of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide and remdesivir exhibited synergy from patient #1 sera in 2 independent experiments and additivity in a single experiment with sera from patient #2 (Table 7). The different classification is most likely due to the different convalescent serum used as detection reagents. These same antiviral data were also analysed using Synergyfinder program, which also indicated that the 2 drugs were additive to synergistic, with a representative graph shown in FIG. 5. Antagonism was not demonstrated for the combination of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide and remdesivir in these studies. Serial dilutions of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide (designated PF-00835231 in FIG. 5) and remdesivir (concentrations are shown along axis in FIG. 5) were combined in a matrix format. A 3-dimensional drug interaction landscape plotting synergy scores analyzed using GeneData program across all concentrations tested (median scores of three replicates) are shown in FIG. 5. Area of the scores above the plain in the 3-dimensional graph indicates synergism, while under the plain indicates antagonism. The observed additivity/synergy was not due to cytotoxicity, as there was no noticeable cytotoxicity in virus infected host cells for all the combinations tested.

TABLE 7
Combination Synergy Score of N-((1S)-1-{[((1S)-3-
hydroxy-2-oxo-1-{[(3S)-2-oxopyrrolidin-3-yl]methyl}
propyl)amino] carbonyl}-3-methylbutyl)-4-methoxy-
1H-indole-2-carboxamide with Remdesivir

| | Loewe | Bliss | HSA | | |
|---|---|---|---|---|---|
| Patient | Synergy | Synergy | Synergy | Combination | |
| Sera | Score | Score | Score | Index | n |
| 1 | 1.60 | 1.60 | 2.51 | 0.860 | 2 |
| | (1.18; 2.02) | (1.59; 1.60) | (2.12; 2.89) | (0.837; 0.882) | |
| 2 | (−0.0776) | (0.366) | (0.830) | (1.04) | 1 |

HSA = Highest single agent;
n = number of determinations;
Data shows average; (individual values)

FIG. 5A provides a graphical representation of the activity of N-((1S)-1-{[((1S)-3-hydroxy-2-oxo-1-{[(3S)-2-oxopyrrolidin-3-yl]methyl} propyl)amino] carbonyl}-3-methylbutyl)-4-methoxy-1H-indole-2-carboxamide in combination with remdesivir in a HELA-ACE2 cell assay. The EC₉₀ (nM) of N-((1S)-1-{[((1S)-3-hydroxy-2-oxo-1-{[(3S)-2-oxopyrrolidin-3-yl]methyl} propyl)amino] carbonyl}-3-methylbutyl)-4-methoxy-1H-indole-2-carboxamide found in this assay decreased with increasing concentration of remdesivir (i.e. EC90 decreased from 433 nM to 123 nM to 54.5 nM then to <0.78 nM when the remdesivir concentration increased from 0 to 48 to 95 to 190, respectively).

In vitro drug efficacy and cytotoxicity in A549+ACE2 cells. A549+ACE2 cells were seeded into black wall 96-well plates at 70% confluency. The next day, media was removed and replaced with complete media containing compound/carrier two hours prior to infection. Cells were then infected at 0.425 multiplicity of infection (MOI), based on Vero E6 titer, at 37° C. 1 hour post virus addition, virus was removed, and media containing compound/carrier was added. At 24 and 48 hours post infection, cells were fixed by submerging in 10% formalin solution for 30-45 min. After fixation, cells were washed once with H2O to remove excess formalin. Plates were dried and PBS was added per well before exiting the BSL-3 facility. Fixed cells were permeabilized with Triton-X and stained with mouse monoclonal SARS-CoV anti-N antibody 1C7, which cross-reacts with SARS-CoV-2, goat anti-mouse AlexaFluor 647, and DAPI. Plates were scanned on the Celllnsight CX7 LZR high-content screening platform. A total of 9 images were collected at 4× magnification to span the entire well. Images were analyzed using HCS Navigator to obtain total number of cells/well (DAPI stained cells) and percentage of SARS-CoV-2 infected cells (AlexaFluor 647 positive cells). To enable accurate quantification, exposure times for each channel were adjusted to 25% of saturation and cells at the edge of each image were excluded in the analysis. SARS-CoV-2-infected cells were gated to include cells with an average fluorescence intensity greater than 3 standard deviations that of mock infected and carrier treated cells. Representative images of viral foci were acquired using the BZ-X810 at 40×magnification of plates fixed at 48 hpi SARS-CoV-2 infection. For determination of cytotoxicity, A549+ACE2 cells were seeded into opaque white wall 96-724 well plates. The following day, media was removed, replaced with media containing compound/carrier or staurosporine, and incubated for 24 or 48 hours, respectively. At these timepoints, ATP levels were determined by CellTiter-Glo 2.0 (Promega, cat no. G9242) using a BioTek Synergy HTX multi-mode reader.

PF-00835231, when evaluated against SARS-CoV-2 USA-WA1/2020 in A549+ACE2 cells, at 24 hours post infection had an EC50 of 0.221 μM (95% CI 0.137-0.356), an EC90 of 0.734 μM (95% CI 0.391-1.38) and a CC50 of >10 M; at 48 hours post infection had an EC50 of 0.158 μM (95% CI 0.0795-0.314), an EC90 of 0.439 μM (95% CI 0.380-0.508) and a CC50 of >10 M; when evaluated against SARS-CoV-2 USA-NYU-VC-003/2020, at 24 hours post infection had an EC50 of 0.184 μM (95% CI 0.016-0.377), an EC90 of 0.591 μM (95% CI 0.534-0.654) and a CC50 of >10 M.

Human airway epithelial cultures (HAEC). To generate HAEC, Bci-NS1.1 were plated (7.5 E+04 cells/well) on rat-tail collagen type 1-coated permeable transwell membrane supports (6.5 mm; Corning, cat no. 3470), and immersed apically and basolaterally in Pneumacult Ex Plus medium (StemCell, cat no. 05040). Upon reaching confluency, medium was removed from the apical side (“airlift”), and medium in the basolateral chamber was changed to Pneumacult ALI maintenance medium (StemCell, cat no. 05001). Medium in the basolateral chamber was exchanged with fresh Pneumacult ALI maintenance medium every 2-3 days for 12-15 days to form differentiated, polarized cultures that resemble in vivo pseudostratified mucociliary epithelium. Cultures were used within 4-6 weeks of differentiation. HAEC were used for cytotoxicity assays and SARS-688 CoV-2 infections.

Compound acquisition, dilution, and preparation. PF-00835231, remdesivir, and CP-100356 were solubilized in 100% DMSO and provided by Pfizer, Inc. Compound stocks diluted in DMSO to 30 mM were stored at −20° C. Compounds were diluted to 10 μM working concentration in complete media or Pneumacult ALI maintenance medium. All subsequent compound dilutions were performed in according media containing DMSO equivalent to 10 μM compound.

In vitro efficacy and cytotoxicity in human airway epithelial cultures (HAEC). 48 hours prior to infection, 2-6-week-old HAEC were washed apically twice for 30 min each with pre-warmed PBS containing calcium and magnesium, to remove mucus on the apical surface. 2 hours prior to infection, HAEC were pretreated by exchanging the ALI maintenance medium in the basal chamber with fresh medium containing compounds or carrier. Remdesivir and PF-00835231 were used at 10, 0.5 and 0.025 μM, and CP-100356 at 1 μM. 1 hour prior to infection, cultures were washed apically twice for 30 min each with pre-warmed PBS containing calcium and magnesium. Each culture was infected with 1.35E+05 PFU (Vero E6) per culture for 2 hours at 37° C. A sample of the inoculum was kept and stored at −80° C. for back-titration by plaque assay on Vero E6 cells. For assessment of compound toxicity, additional cultures were washed and pretreated as the infected cultures. Instead of being infected, these cultures were incubated with PBS containing calcium and magnesium only as Mock treatment. HAEC were incubated with the viral dilution or Mock treatment for 2 hours at 37° C. The inoculum was removed, and the cultures were washed three times with pre-warmed PBS containing calcium and magnesium. For each washing step, buffer was added to the apical surface and cultures were incubated at 37° C. for 30 min before the buffer was removed. The third wash was collected and stored at −80° C. for titration by plaque assay on Vero E6 cells. Infected cultures were incubated for a total of 72 hours at 37° C. Infectious progeny virus was collected every 12 hours by adding 60 μL of pre-warmed PBS containing calcium and magnesium, incubation at 37° C. for 30 min, and collection of the apical wash to store at −80° C. until titration. Additionally, trans-epithelial electrical resistance (TEER) was measured in uninfected but treated HAEC to quantify the tissue integrity in response to treatment with compounds or carrier. At the end point, cultures were fixed by submerging in 10% formalin solution for 24 hours and washed three times with PBS containing calcium and magnesium before further processing for histology. Alternatively, at the end point, transwell membranes were excised and submerged in RLT buffer to extract RNA using the RNAeasy kit (Qiagen, cat no. 74104). cDNA synthesis was performed using SuperScript™ III system (ThermoFisher cat no. 18080051) followed by RT-qPCR with TaqMan universal PCR master mix (ThermoFisher cat no. 4305719) and TaqMan gene expression assay probes (ThermoFisher GAPDH cat no. 4333764F, BAX cat no. Hs00180269_m1, BCL2 cat no. Hs00608023_m1) using a QuantStudio 3 Real Time PCR System.

For additional determination of cytotoxicity in undifferentiated HAEC precursor cells, Bci-NS1.1 cells were seeded into opaque white wall 96-well plates. The following day, media was removed, replaced with media containing compound/carrier or staurosporine, and incubated for 24 or 48 hours, respectively. At these timepoints, ATP levels were determined by CellTiter-Glo 2.0 (Promega, cat no. G9242) using a BioTek Synergy HTX multi-mode reader.

The PF-00835231 anti-SARS-CoV-2 activity in HAEC, is assessed at either 0.025, 0.5 or 10 μM PF-00835231 or remdesivir, or DMSO carrier control, to the basolateral chamber of HAEC. HAEC is apically challenged with SARS-CoV-2 USA-WA1/2020, and viral infectious titers are determined from apical washes collected at 12-hour increments. Progeny viral particles in apical washes from DMSO-treated cultures are present at 12 hours post infection, indicating that the SARS-CoV-2 life cycle in HAEC cells is completed by that time. Both PF-00835231 and remdesivir potently inhibit SARS-CoV-2 titers in a dose-dependent manner, with the 10 μM doses resulting in viral titers below the limit of 366 detection at most time points.

Favorable preclinical ADME and pharmacokinetic profile of N-((1S)-1-{[((1S)-3-hydroxy-2-oxo-1-{[(3S)-2-oxopyrrolidin-3-yl]methyl} propyl)amino] carbonyl}-3-methylbutyl)-4-methoxy-1H-indole-2-carboxamide

The metabolic stability of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide was evaluated in vitro using pooled human liver microsomes (HLM) and hepatocytes. The drug was shown to be metabolized by cytochrome P450 enzymes exhibiting an unbound CIᵢₙₜ 14 μl/min/mg. With the use of chemical inhibitors and recombinant heterologously expressed enzymes, CYP3A4 was identified as the major CYP involved in the metabolism of this compound. It was also noted that the polymorphically expressed CYP3A5 can also metabolize N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide and that clearance may be slightly greater in CYP3A5 expressers. The potential for the compound to reversibly inhibit human cytochrome P450 enzymes (CYP1A2, 2B6, 2C8, 2C9, 2C19, 2D6, and 3A) was evaluated using probe substrates (supplemental) in pooled HLM and provided IC₅₀ values >200 μM and a weak signal for time dependent inhibition of CYP3A4/5 indicating N-((1S)-1-{[((1S)-3-hydroxy-2-oxo-1-{[(3S)-2-oxopyrrolidin-3-yl]methyl} propyl)amino] carbonyl}-3-methylbutyl)-4-methoxy-1H-indole-2-carboxamide provides a low risk of causing drug-drug interactions (DDI) on coadministration with other drugs. The potential for N-((1S)-1-{[((1S)-3-hydroxy-2-oxo-1-{[(3S)-2-oxopyrrolidin-3-yl]methyl} propyl)amino] carbonyl}-3-methylbutyl)-4-methoxy-1H-indole-2-carboxamide to inhibit a range of transporters (BCRP, Pgp, OATP1B1/1B3, OCT1/2, OAT1/3 and MATE1/2K) was evaluated using in vitro systems. The IC₅₀ values >20 μM indicating a low risk of causing DDI's due to transporter inhibition at the projected clinical exposure. The plasma protein binding of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide was measured across species using equilibrium dialysis showing moderate binding to plasma proteins with plasma free fractions of 0.26 to 0.46 across species.

N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide was administered intravenously to rats, dogs and monkeys (1 or 2 mg/kg) and exhibited moderate plasma clearances (35-60% liver blood flow), low volumes of distribution (<1 L/Kg) and short half-lives (<1.5 h) across species in keeping with its neutral physiochemistry and lipophilicity (SFLogD_{7.4}=1.7). Following oral administration to rats (2 mg/kg) and monkeys (5 mg/kg) N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide exhibited low bioavailability (<2%), likely due to a combination of low absorption because of its low permeability (apparent MDCK-LE permeability of 1.3×10⁻⁶ cm/sec^{28,34}), low solubility, potential for active efflux in the gut by P-gp and BCRP, as well as the potential for amide hydrolysis by digestive enzymes in the gastrointestinal tract. In rat, dog and monkey approximately 10% of N-((1S)-1-{[((1S)-3-hydroxy-2-oxo-1-{[(3S)-2-oxopyrrolidin-3-yl]methyl} propyl)amino] carbonyl}-3-methylbutyl)-4-methoxy-1H-indole-2-carboxamide was eliminated unchanged in the urine indicating renal elimination may also play a minor role in the clearance of N-((1S)-1-{[((1S)-3-hydroxy-2-oxo-1-{[(3S)-2-oxopyrrolidin-3-yl]methyl} propyl)amino] carbonyl}-3-methylbutyl)-4-methoxy-1H-indole-2-carboxamide in humans.

Human pharmacokinetic predictions suitable for IV administration—taking into account the human in vitro metabolism data and in vivo pharmacokinetic (PK) data in rats, dogs and monkeys N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide is predicted to exhibit a plasma clearance (CLₚ) of ˜6 mL/min/kg (major CYP, minor renal pathways) steady state volume of distribution (V_{dss}) of 1 L/kg and half-life of approximately 2 h in humans. Due to the limited oral bioavailability, short elimination half-life, and the likely need to maintain free systemic concentrations over time, a continuous intravenous (IV) infusion was proposed as the optimal dosing route and regimen.

### Efficacious Target Concentration and Feasible Human Dose Projection to Achieve Target Ceff

The inhibitory quotient (IQ) has been a useful metric for translating preclinical antiviral potencies to the clinic across a number of viral diseases. IQ is defined as the human C_{min,u} unbound concentration divided by the in vitro unbound (serum adjusted) EC_{50,u} value in the antiviral assay (equation 1).

$$I Q = \frac{{C}_{min , u}}{{EC}_{50 , u}} \qquad ( 1 )$$

Some antiviral therapies have shown significant benefit with IQ close to 1; however, rapidly controlling viral replication frequently requires maintaining an exposure at least 10× higher than in vitro EC₅₀. Clinically approved protease inhibitors have effectively decreased viral loads when dosed at IQ values from 1-100, when protein binding and site of action exposure are taken into account. Importantly, antivirals in general and, specifically, protease inhibitors can potentially lead to increased mutations and additional drug resistance when dosed at an IQ less than 1.

How high an IQ value is required depends on the slope of the dose response curve. The Hill coefficient (m) and the EC₅₀ are related to the in vitro antiviral activity at a range of concentrations (C) by equation 2:

$$in vitro antiviral activity = 100 * \frac{{C}^{m}}{E {C}_{50}^{m} + {C}^{m}} \qquad ( 2 )$$

N-((1S)-1-{[((1S)-3-hydroxy-2-oxo-1-{[(3S)-2-oxopyrrolidin-3-yl]methyl} propyl)amino]carbonyl}-3-methylbutyl)-4-methoxy-1H-indole-2-carboxamide shows a high slope (m=3) across a range of in vitro antiviral assays, like those of clinical protease inhibitors targeting HIV and HCV. There is only a 2-to-3-fold difference between the antiviral EC₅₀ and EC₉₀ concentrations, rather than the typical 9-fold difference for antiviral agents with Hill coefficients of 1. Therefore, relatively small ratios of exposure to EC₅₀ values (3-10) are related to near complete viral suppression.

The projected minimally efficacious concentration (C_{eff}) was chosen to match the in vitro EC₉₀, consistent with the preclinical to clinical translation of approved protease inhibitors. Since N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide was proposed to be administered by continuous infusion, the projected steady state exposure is equal to the Cₘᵢₙ maintained over the dosing interval. The dose response assay performed in the physiologically relevant cell type, human lung carcinoma, resulted in an average EC₉₀ value of 0.44 μM. This is consistent with additional antiviral data in Hela-ACE2 cells (EC₉₀=0.4 μM) and Vero-cell lines (EC₉₀=−0.48-1.6 μM) when a P-gp inhibitor was added to better reflect the lack of substantial P-gp transporter in the lung. Furthermore, the antiviral inhibition is supported by the antiviral time course experiment performed in a primary human airway epithelial model (preliminary data indicates an unbound EC₉₀<0.5 μM), indicating a consistent intrinsic anti-SARS-CoV-2 activity of N-((1S)-1-{[((1S)-3-hydroxy-2-oxo-1-{[(3S)-2-oxopyrrolidin-3-yl]methyl}propyl)amino] carbonyl}-3-methylbutyl)-4-methoxy-1H-indole-2-carboxamide across different cell types. Therefore, the proposed target C_{eff} is ˜0.5 μM.

Due to the rapid blood perfusion through the lungs and the continuous steady state intravenous infusion regimen, the free plasma and free lung concentrations are assumed to be in equilibrium and, therefore, the free plasma concentration provides a reasonable surrogate for the concentration at the main site of action of the disease. Based on the human PK predictions, the minimally efficacious dose of N-((1S)-1-{[((1S)-3-hydroxy-2-oxo-1-{[(3S)-2-oxopyrrolidin-3-yl]methyl} propyl)amino] carbonyl}-3-methylbutyl)-4-methoxy-1H-indole-2-carboxamide necessary to achieve this exposure is 320 mg/day administered as an intravenous continuous infusion. The required duration of dosing for efficacy remains uncertain and will need to be evaluated in humans. Based on clinical results from remdesivir a duration of up to 10 days of dosing may be required to provide improved patient outcomes.

All patents and publications described hereinabove are hereby incorporated by reference in their entirety. While the invention has been described in terms of various preferred embodiments and specific examples, the invention should be understood as not being limited by the foregoing detailed description, but as being defined by the appended claims and their equivalents.

1.-43. (canceled)

44. A method of treating COVID-19 or for inhibiting or preventing SARS-COV-2 replication in a patient, the method comprising administering N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof in combination with remdesivir and/or azithromycin to the patient in need of treatment thereof.

45. The method according to claim 44 wherein the N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof is administered orally or intravenously.

46. The method according to claim 45 wherein the N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof is administered intravenously.

47. The method according to claim 46 wherein the N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof is administered intermittently over a 24-hour period or continuously over a 24-hour period.

48. The method according to claim 44 wherein 0.2 g/day to 4 g/day of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof is administered.

49. The method according to claim 48 wherein 0.3 g/day to 3 g/day of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof is administered.

50. The method according to claim 44 wherein N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof is administered by continuous intravenous infusion.

51. The method according to claim 50 wherein about 0.3 g/day to 3 g/day of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof is administered by continuous intravenous infusion.

52. The method according to claim 51 wherein the N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof is administered by continuous intravenous infusion in an amount sufficient to maintain an effective concentration (C_{eff}) of approximately 0.5 μM.

53. The method according to claim 44 wherein remdesivir is co-administered with N-((1S)-1-{[((1S)-3-hydroxy-2-oxo-1-{[(3S)-2-oxopyrrolidin-3-yl]methyl}propyl) amino]carbonyl}-3-methylbutyl)-4-methoxy-1H-indole-2-carboxamide or a pharmaceutically acceptable salt thereof by continuous intravenous infusion.

54. The method according to claim 44, wherein the N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide is a hydrate (Form 3) having a powder X-ray diffraction pattern comprising three or more X-ray diffraction peaks, in degrees 2-theta, selected from 8.6±0.2, 11.9±0.2, 14.6±0.2, 18.7±0.2 and 19.7±0.2.

55. The method according to claim 54 wherein the powder X-ray diffraction peaks, in degrees 2-theta, are 14.6±0.2, 18.7±0.2 and 19.7±0.2.

56. The method according to claim 54 wherein the powder X-ray diffraction peaks, in degrees 2-theta, are 8.6±0.2, 14.6±0.2, 18.7±0.2 and 19.7±0.2.

57. The method according to claim 54 wherein the powder X-ray diffraction peaks, in degrees 2-theta, are 8.6±0.2, 11.9±0.2, 14.6±0.2, 18.7±0.2 and 19.7±0.2.

58. The method according to claim 44 wherein the N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof is formulated into an aqueous liquid composition comprising:
a. from about 0.2 mg/mL to about 2.0 mg/mL of the N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof;
b. one or more co-solvents;
c. optionally one or more surfactants; and
d. a buffer,
wherein the aqueous liquid composition has a pH of about 1.5 to about 6 and the total amount of the one or more co-solvents is up to about 30% (v/v).

59. The method according to claim 58 wherein the aqueous liquid composition is administered intravenously in a volume of about 1000 mL or less per day, has a pH of about 3 to about 5 and the total amount of the one or more co-solvents is up to about 20% (v/v).

60. The method according to claim 59 wherein the aqueous liquid composition comprises:
a. about 0.2 mg/mL to about 1.0 mg/mL of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof;
b. the one or more co-solvents are selected from the group consisting of benzyl alcohol (BA), dimethylacrylamide (DMA), dimethyl sulfoxide (DMSO), ethanol, N-methyl pyrrolidone (NMP), polyethylene glycol and propylene glycol (PG), wherein the total amount of the one or more co-solvents is up to about 10% (v/v);
c. the one or more surfactants, when present, are selected from the group consisting of polyvinylpyrrolidone (PVP), poloxamer 407, poloxamer 188, hydroxypropyl methylcellulose (HPMC), polyethoxylated castor oil, lecithin, polysorbate 80 (PS80), polysorbate 20 (PS20) and polyethylene glycol (15)-hydroxystearate; and
d. the buffer is selected from the group consisting of acetic acid, citric acid, lactic acid, phosphoric acid and tartaric acid.

61. The method according to claim 60 wherein the aqueous liquid composition comprises:
a) one or two co-solvents selected from the group consisting of dimethyl sulfoxide (DMSO), ethanol, PEG300 and PEG400;
b) one or two surfactants, when present, selected from polysorbate 80, polysorbate 20, and polyethylene glycol (15)-hydroxystearate; and
c) a citric acid buffer of up to 50 mM.

62. The method according to claim 61 wherein the aqueous liquid composition is administered by continuous intravenous infusion in a volume of about 250 mL to about 500 mL per day.

63. The method according to claim 44 wherein the N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof is formulated into a pharmaceutical composition comprising:
a. a therapeutically effective amount of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof;
b. a complexing agent selected from the group consisting of α-cyclodextrins, β-cyclodextrins, γ-cyclodextrins, nicotinamide, sodium benzoate and sodium salicylate, wherein the molar ratio of the complexing agent to the Compound 1 is from about 1.5:1 to about 25:1; and
c. a buffer.

64. The method according to claim 63 wherein the pharmaceutical composition is a ready to use or ready to dilute parenteral solution, which:
a. optionally comprises one or more co-solvents which are selected from the group consisting of benzyl alcohol (BA), dimethylacrylamide (DMA), dimethyl sulfoxide (DMSO), ethanol, N-methyl pyrrolidone (NMP), polyethylene glycol and propylene glycol (PG);
b. further optionally comprises a surfactant which is selected from the group consisting of polyvinylpyrrolidone (PVP), poloxamer 407, poloxamer 188, hydroxypropyl methylcellulose (HPMC), polyethoxylated castor oil, lecithin, polysorbate 80 (PS80), polysorbate 20 (PS20) and polyethylene glycol (15)-hydroxystearate; and
c. comprises a buffer selected from the group consisting of acetic acid, citric acid, lactic acid, phosphoric acid and tartaric acid;
wherein the pH of the parenteral solution is about 3 to about 5.

65. The method according to claim 64 wherein the pharmaceutical composition comprises:
a. the N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof at a concentration of about 0.2 mg/mL to about 16 mg/mL;
b. the complexing agent selected from the group consisting of hydroxypropyl-β-cyclodextrin (HP-β-CD) and sulfobutylether-β-cyclodextrin (SBE-β-CD), wherein the molar ratio of the complexing agent to the Compound 1 is from about 1.5:1 to about 8:1;
c. one or two co-solvents selected from the group consisting of dimethyl sulfoxide (DMSO), ethanol, PEG300, PEG400 and propylene glycol, wherein the total concentration of the co-solvents is up to about 15% (v/v) of the pharmaceutical composition;
d. optionally a surfactant selected from the group consisting of polysorbate 80 (PS80), polysorbate 20 (PS20) and polyethylene glycol (15)-hydroxystearate; and
e. a citric acid buffer.

66. The method according to claim 65 wherein the pharmaceutical composition comprises:
a. the N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof at a concentration of about 0.2 mg/mL to about 8 mg/mL;
b. the complexing agent selected from the group consisting of hydroxypropyl-β-cyclodextrin (HP-β-CD) and sulfobutylether-β-cyclodextrin (SBE-β-CD), wherein the molar ratio of the complexing agent to the N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof is from about 2:1 to about 6:1; and
c. one or two co-solvents selected from the group consisting of dimethyl sulfoxide (DMSO), ethanol, PEG300, PEG400 and propylene glycol, wherein the total concentration of the co-solvents is up to about 10% (v/v) of the pharmaceutical composition.

67. The method according to claim 66 wherein the pharmaceutical composition comprises about 1.1% ethanol (v/v), about 3.4% PEG400 (v/v), about 80 mg/mL SBE-pi-cyclodextrin, about 6 mg/mL the Compound 1; up to about 50 mM citric acid, wherein the pH is about 4 to about 5.

68. The method according to claim 63 wherein the pharmaceutical composition is a lyophile or powder ready for reconstitution into a solution suitable for parenteral administration.

69. The method according to claim 68 wherein the N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide is a hydrate (Form 3).

70. The method according to claim 44 wherein the N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof is formulated into an aqueous liquid composition comprising:
a) from about 0.2 mg/mL to about 16 mg/mL of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof;
b) a complexing agent;
c) a buffer;
d) optionally one or more co-solvents; and
e) optionally one or more surfactants; wherein the aqueous liquid composition has a pH of about 1.5 to about 6, and the total amount of the one or more co-solvents, when present, is up to about 15% (v/v) of the aqueous liquid composition.

71. The method according to claim 70 wherein the aqueous liquid composition:
a) is administered intravenously in a volume of about 1000 mL or less per day;
b) has a pH of about 3 to about 5; and
c) has a total amount of the one or more co-solvents, when present, up to about 10% (v/v) of the aqueous liquid composition.

72. The method according to claim 71 wherein the aqueous liquid composition comprises: a) about 0.2 mg/mL to about 8.0 mg/mL of N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof;
b) a complexing agent selected from the group consisting of α-cyclodextrins, β-cyclodextrins, γ-cyclodextrins, nicotinamide, sodium benzoate and sodium salicylate, where the molar ratio of the complexing agent to N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof is from about 1.5:1 to about 25:1;
c) a buffer selected from the group consisting of acetic acid, citric acid, lactic acid, phosphoric acid and tartaric acid;
d) one or more co-solvents selected from the group consisting of benzyl alcohol (BA), dimethylacrylamide (DMA), dimethyl sulfoxide (DMSO), ethanol, N-methyl pyrrolidone (NMP), polyethylene glycol and propylene glycol (PG), wherein the total amount of the one or more co-solvents, when present, is up to about 6% (v/v) of the aqueous liquid composition; and
e) optionally one or more surfactants selected from the group consisting of polyvinylpyrrolidone (PVP), poloxamer 407, poloxamer 188, hydroxypropyl methylcellulose (HPMC), polyethoxylated castor oil, lecithin, polysorbate 80 (PS80), polysorbate 20 (PS20) and polyethylene glycol (15)-hydroxystearate.

73. The method according to claim 72 wherein:
a) the complexing agent is a β-cyclodextrin;
b) the molar ratio of β-cyclodextrin to the N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof is from about 1.5:1 to about 8:1; and
c) the buffer is citric acid up to about 50 mM.

74. The method according to claim 73 wherein:
a) the aqueous liquid composition comprises one to two co-solvents selected from the group consisting of dimethyl sulfoxide (DMSO), ethanol, PEG300, PEG400 and propylene glycol (PG);
b) the complexing agent is selected from the group consisting of hydroxypropyl-β-cyclodextrin (HP-β-CD) and sulfobutylether-β-cyclodextrin (SBE-β-CD); and
c) the surfactant, when present, is selected from polysorbate 80, polysorbate 20 and polyethylene glycol (15)-hydroxystearate.

75. The method according to claim 74 wherein:
a) the two co-solvents are one of ethanol and dimethyl sulfoxide (DMSO), and the other co-solvent is selected from the group consisting of PEG300, PEG400, and propylene glycol (PG), wherein the ratio of ethanol or DMSO to PEG 300, PEG400 or PG is from about 1:2 to about 1:4; or the two co-solvents are ethanol and DMSO, wherein the ratio of ethanol to DMSO is from about 1:2 to about 1:4;
b) the molar ratio of hydroxypropyl-β-cyclodextrin (HP-β-CD) or sulfobutylether-β-cyclodextrin (SBE-β-CD) to the N-((1S)-1-{[((1S)-3-hydroxy-2-oxo-1-{[(3S)-2-oxopyrrolidin-3-yl]methyl}propyl)amino]carbonyl}-3-methylbutyl)-4-methoxy-1H-indole-2-carboxamide or a pharmaceutically acceptable salt thereof is from about 2:1 to about 6:1; and
c) the concentration of the citric acid buffer is up to about 50 mM.

76. The method according to claim 75 wherein the aqueous liquid composition is suitable for continuous intravenous infusion and the volume of the aqueous liquid composition is from about 250 mL to about 500 mL.
